(19) United States
(12) Patent Application Publication
(10) Pub. No.: US 2023/0248835 A1
(43) Pub. Date: Aug. 10, 2023

(54) COMPOUNDS, COMPOSITIONS, AND METHODS FOR THE TREATMENT OF FIBROTIC DISEASES AND CANCER

(71) Applicant: Purdue Research Foundation, West Lafayette, IN (US)

(72) Inventors: Philip S. LOW, West Lafayette, IN (US); John Victor NAPOLEON, West Lafayette, IN (US); Fenghua ZHANG, West Lafayette, IN (US)

(21) Appl. No.: 18/004,656

(22) PCT Filed: Jul. 7, 2021
(86) PCT No.: PCT/US2021/040736

§ 371 Date: Jan. 6, 2023

Related U.S. Application Data
(60) Provisional application No. 63/049,556, filed on Jul. 8, 2020.

(51) Int. Cl. A61K 47/55; C07D 471/04; A61P 35/00

(52) U.S. Cl. CPC A61K 47/551; C07D 471/04; A61P 35/00

(57) ABSTRACT

Compounds, including Toll-like receptor (TLR) agonists, e.g., TLR7 and TLR7/8 agonists and their folic acid or pteroyl amino acid conjugates, and use thereof to treat a cancer or a fibrotic disease or disorder; and methods of making conjugates comprising targeting ligands of folic acid receptor and TLR7 and TLR7/8 agonists.

### PRIORITY

This application is related to and claims the priority benefit of U.S. Provisional Application No. 63/049,556, which was filed Jul. 8, 2020. The content of the aforementioned application is hereby incorporated by reference in its entirety.

### TECHNICAL FIELD

This disclosure relates to compounds, compositions and methods for the treatment of various diseases, including, e.g., cancer, fibrosis, and other disease states. In some embodiments, the present disclosure relates to Toll-like receptor (TLR) agonists, e.g., TLR-7/8 agonists and folate or pteroyl amino acid conjugates thereof, and the use thereof to treat cancer and inflammatory diseases (e.g., fibrotic diseases). In some embodiments, the present disclosure further relates generally to methods of making conjugates comprising targeting ligands of folic acid receptor and TLR 7/8 agonists.

### BACKGROUND

Macrophages are key cellular components of the innate immunity. M1-type macrophages are pro-inflammatory while M2-type macrophages are anti-inflammatory cells. Overstimulation of M1-like and M2-like macrophages has been linked to several diseases, such as fibrosis, inflammatory diseases and cancer. In cancer, tumor-associated macrophages (TAMs) with M2 phenotype represent the most prominent cells in the tumor microenvironment (TME). Increased TAM infiltration has been associated with worse prognosis in many cancers. Within the TME, TAMs contribute to immunosuppressive function. In fibrotic diseases, activated M2-type macrophages produce profibrotic cytokines that induce myofibroblasts to produce extracellular matrix proteins including collagen and fibronectin. Reprogramming these immunosuppressive phenotypes into more pro-inflammatory ones could offer an effective treatment to such diseases.

Toll-like receptors (TLRs) can recognize pathogens and are significantly expressed in immune cells. Synthetic small molecule agonists that target TLR-7/8 are known to function as powerful immunostimulants. However, systemic administration of such TLR-7/8 agonists in a non-targeted form can be hampered with dose-limiting toxicity and cause a toxic cytokine syndrome in humans. Therefore, many of these drugs are applied topically.

### SUMMARY

Provided in certain embodiments herein are compounds and compositions, such as for use in therapeutic methods. In some instances, the compounds and compositions are used in methods of treatment, such as the treatment of cancer and/or fibrosis. In some embodiments, the compounds are Toll-like receptor (TLR) 7 and/or 8 agonists. In certain embodiments, the compounds are used alone or in conjunction with a targeting agent.

A compound provided herein can comprise a first radical. The first radical can be linked (e.g., directly or via a linker) to a second radical. The second radical can be a targeting moiety that targets a pattern recognition receptor of a cell (e.g., an immune cell receptor, such as a folate receptor, such as folate receptor beta (FR-β)). In some embodiment, the targeting ligand comprises a folate receptor binding ligand, such as a folate or a functional fragment or analog thereof, e.g., a pteroyl amino acid (e.g., pteroyl linked to an amino acid or peptide comprising two or more amino acids).

In some instances, such as when the compound is a (e.g., potent) TLR 7/8 agonist, the non-conjugated compound can be highly toxic when delivered systemically. It is desirable to reduce and/or eliminate systemic toxicity associated with such compounds. A conjugated radical of a compound can have reduced toxicity relative to the free form of the compound (e.g., reduced by at least 10%, at least 20%, at least 30%, at least 50%, at least 75%, or at least 90%). Moreover, in some instances, a compound (conjugate) can be efficacious at a comparable or lower concentration (e.g., having an ED50 concentration of 120% of the free form or less, at 100% or less, at 80% or less, at 60% or less, or at 40% or less) relative to a free form of the compound.

In certain embodiments, a compound comprises a first radical connected to a second radical via a non-releasable linkage, such as via a non-releasable linker. Non-releasable linkage of a compound or analog thereof in a conjugate can reduce systemic exposure (e.g., corresponding toxicity) to the compound.

In certain embodiments, the second (e.g., targeting) radical is folate or an analog, functional fragment, or derivative thereof (e.g., a pteroyl amino acid). In some instances, such ligands are useful for targeting a pattern recognition receptor of a cell, such as FR-β. In some instances, FR-β is overexpressed in activated myeloid cells, while being present in extremely low levels in healthy cells.

In some embodiments, a first radical provide herein is an agonist, e.g., potent agonist, of TLR 7 and/or 8 (TLR 7/8). In some embodiments, delivering TLR-7/8 agonists via a targeting ligand (e.g., folate or pteroyl amino acid) is demonstrated to be effective in mitigating systemic cytokine release.

In some embodiments, TLR-7/8 agonists conjugated with folate provide specificity for diseased cell types. In one embodiment, folate-TLR7/8 agonist conjugates can be delivered (e.g., specifically) into the endosome of FR-β+ macrophages, e.g., while limiting systemic exposure to the TLR7/8 agonists.

In some instances, direct alkylation of tertiary hydroxyl with alkyl halides generally proceeds with lower yields and regioisomeric products due to steric effects of the bulky tert-butyl group. In some embodiments, compounds comprising radicals (e.g., TLR7 agonist radicals) are coupled to folate (radical) with greater efficiency as compared with more sterically obstructed compounds. In some embodiments, an alkylene spacer (e.g., n>0, such as n=1-8, such as n=1, forming a methylene) is positioned between a tert-butyl group and a hydroxyl (e.g., as illustrated in Formula I below), allowing for an efficient chemical synthesis and yielding stable conjugates in high yields.

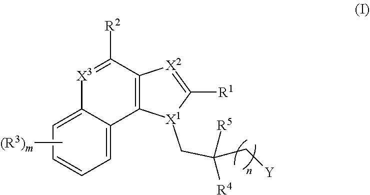

An advantage of such compounds is that, in some instances, such compounds (radicals thereof) form stable conjugates with folate ligands or functional fragments or analogs thereof connected via a non-releasable linker. In some embodiments, such as when Y is a hydroxyl, connection to a linker results in formation of an ester (—OCO—), a carbonate (—OC(═O)O—), or a carbamate (—OC(═O)NR—) at Y. In other instances, Y is another group as described herein. In some embodiments, incorporation of a spacer enables conjugates having a radical of formula (I) connected to a folate receptor ligand via a non-releasable linker to form stable adducts. In some instances, the conjugates are more stable (e.g., in vivo), reducing the systemic exposure of TLR7 agonist and, e.g., reducing adverse effects and side effect profiles.

In some embodiments provided herein is a compound represented by (or comprising a radical of) the structure of Formula (I):

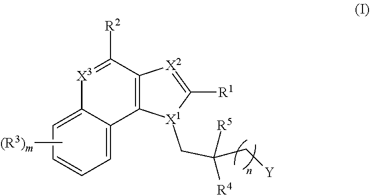

wherein:
R¹, R³, R⁴, R⁵ are each independently a H, an alkyl, an alkoxyl, an alkenyl, an alkynyl, a cycloalkyl, an alicyclic, an aryl (e.g., a biaryl), a halo, a heteroaryl, —COR^{2x},

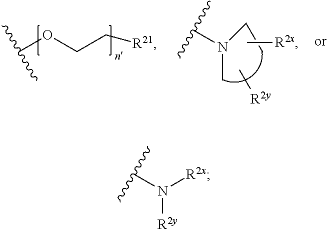

R² is a H, —OH, —NH₂, —NHR^{2x}, N₃, —NH—CH₂—NH₂, —CONH₂, —SO₂NH₂, —NH—CS—NH₂,

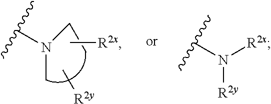

Y is a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

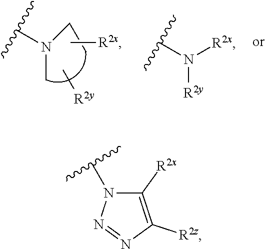

wherein:
each of R^{2x} and R^{2y} is independently selected from a group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each of R^{2z} is independently selected from a group consisting of —NH₂, —NR^{2q}R^{2q′}—O—R^{2q}, —SO—R^{2q}, and —COR^{2q},
wherein each R^{2q} and R^{2q′} is independently alkyl or H

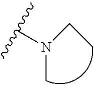 [SMILES: *N(C)CC]

is a 3-10 membered N-containing non-aromatic, mono- or bicyclic heterocycle;
each of X¹, X², and X³ is independently CR^{q} or N;

R²¹ is H or alkyl; and

n′ is 0-30;

wherein, in Formula I, each of each of X¹, X², and X³ is independently CR^{q} or N, where each R^{q} is independently hydrogen, halogen, or optionally substituted alkyl;
n is 0-30 (e.g., 1-8 or 1-6); m is 0-4; and
wherein when n is 0, Y is not H, —OH, or —O—R^{2x}.

In some embodiments, the compound of Formula I is substituted with one or more R³ group(s) (e.g., m R³ groups, such as wherein m is 0-4).

Certain embodiments provide for a compound having (or comprising a radical of) the structure of Formula (IA):

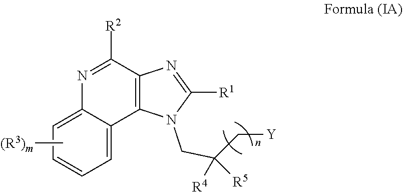

or a pharmaceutically acceptable salt thereof, wherein:
R¹ is an optionally substituted C₃-C₈ alkyl (e.g., acyclic or cyclic) (e.g., optionally substituted with one or more substituent(s), each substituent independently being halogen, alkyl, heteroalkyl, alkoxy, or cycloalkyl);
R² is H, —OR^{z}, —SO₂N(R^{z})₂, —NR^{2x}R^{2y}, or N₃;

Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, where:

R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituent(s), each substituent independently being oxo, halogen, alkyl, heteroalkyl, alkoxy, or cycloalkyl), where each R^{z} is independently hydrogen, halogen, or optionally substituted alkyl, or

R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);

each R³ is independently halogen, —N₃, —CN, —NO₂, —COR^{z}, —COOR^{z}, —CON(R^{z})₂, —COSR^{z}, —SO₂N(R^{z})₂, —CON(R^{z})₂, alkyl, heteroalkyl, cycloalkyl, heterocycloalkyl, alkoxy, amino, hydroxy or thiol, wherein the alkyl, alkoxy, heteroalkyl, cycloalkyl, or heterocycloalkyl is optionally substituted;

R⁴ and R⁵ are each independently alkyl, alkoxy, halogen, or cycloalkyl, wherein the alkyl, alkoxy, or cycloalkyl is optionally substituted;
n is 1-6; and
m is 0-4.

In some embodiments, R¹ of Formula (I) or (IA) is an optionally substituted C₃-C₆ alkyl. In one embodiment, R¹ of Formula (I) or (IA) is an optionally substituted acyclic C₃-C₆ alkyl.

In some embodiments of Formula (I) and Formula (IA), R² is —NR^{2x}R^{2y}. In one embodiment of Formula (I) or (IA), R² is NH₂.

In some embodiments, the compound is represented by (or comprising a radical of) any one or more of the formulae:

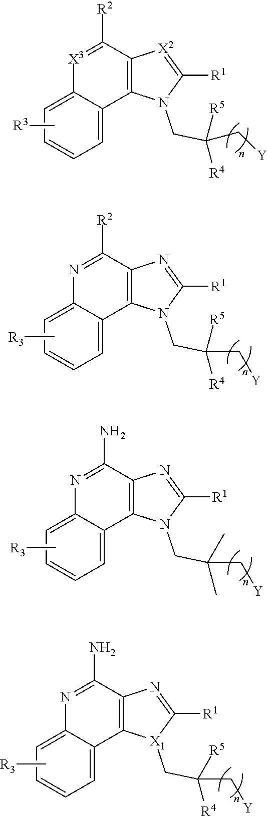

or a pharmaceutically acceptable salt thereof.

In some embodiments, n is 1-3. In another embodiment, n is 1 or 2. In certain embodiments of the compounds hereof, n is 1.

One embodiment provides a compound, or a pharmaceutically acceptable salt thereof, having the structure of Formula (I) or (IA) wherein Y is —OH, OCH₃, —NH₂, —NHNH₂, —NHCONH₂, —SH, —SO₂NH₂, —N₃, —COOH, —COCH₃, —COOCH₃, or —CONH. In some embodiment, Y is OH. In other embodiments, Y is NH₂.

In some embodiments, the compound is represented by (or comprises a radical of) any one or more of the formulae:

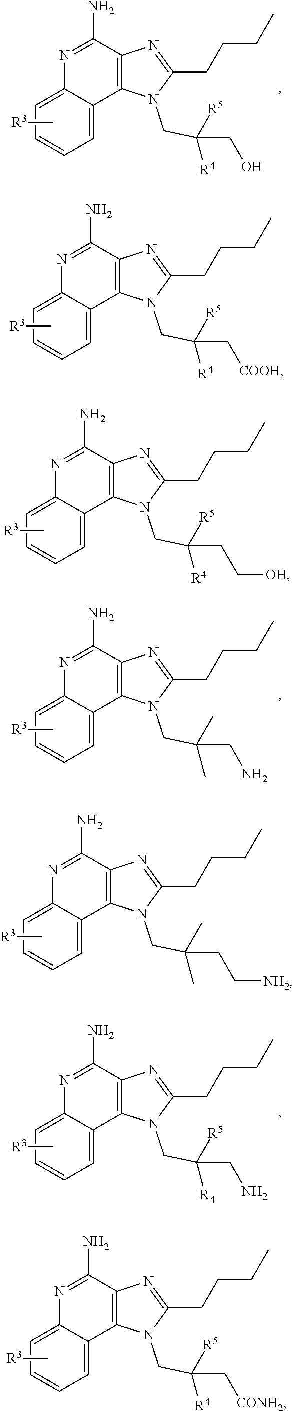

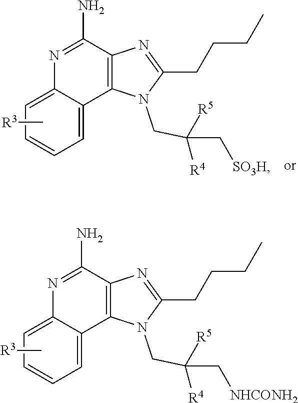

or a pharmaceutically acceptable salt thereof.

In some embodiments of Formula (I) or (IA), R⁴ and R⁵ are each alkyl. In certain embodiments of Formula (I) or (IA), R⁴ and R⁵ are each independently C₁-C₄ alkyl. In one embodiment, R⁴ and R⁵ are each methyl.

In some embodiments, the compound is represented by (or comprises a radical of) any one of the following formulae:

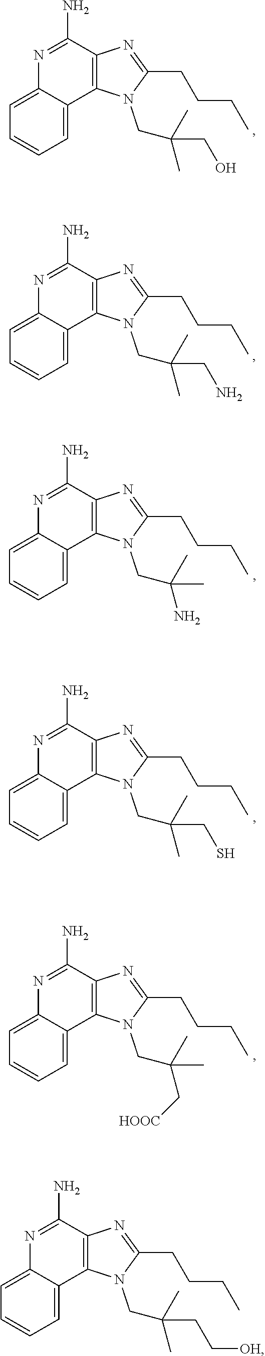

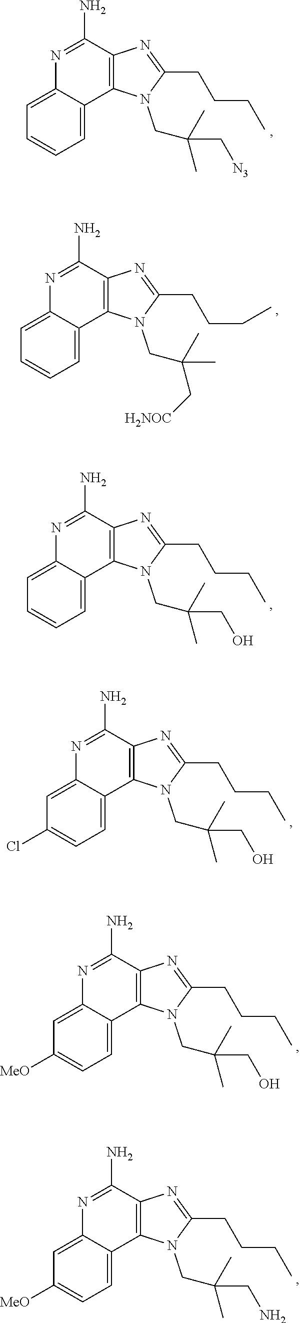

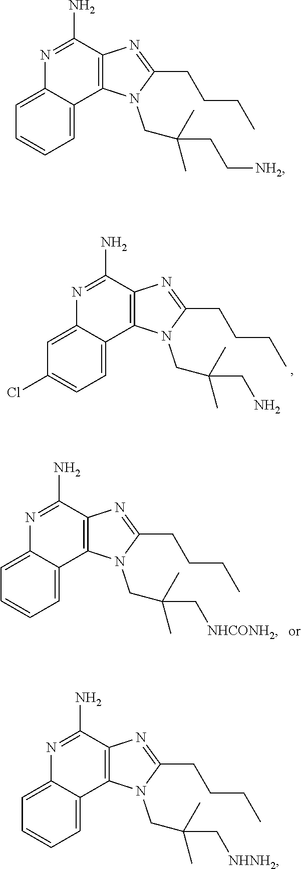

or a pharmaceutically acceptable salt thereof.

In some embodiments, X¹, X², and X³ are each N.

In certain embodiments of Formula (I) or (IA), the compound is of the formula:

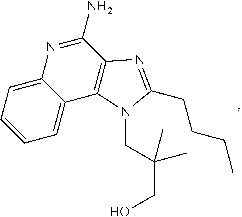 [SMILES: CCCCc1nc2c(N)nc3ccccc3c2n1CC(C)(C)CO]

or a pharmaceutical salt thereof.

One embodiment provides a compound represented by the structure of Formula (II):

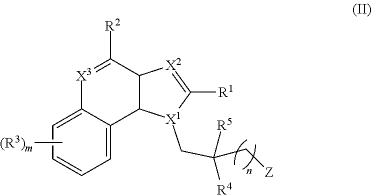

or a pharmaceutically acceptable salt thereof, wherein:
R¹, R³, R⁴, R⁵ are each independently a H, an alkyl, an alkoxyl, an alkenyl, an alkynyl, an alicyclic, an aryl, a biaryl, a halo, a heteroaryl, —COR^{2x},

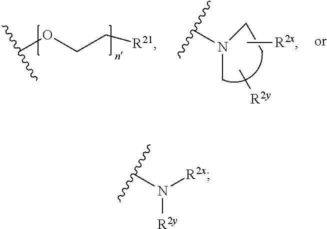

R² is a H, —OH, —NH₂, —NHR^{2x}, N₃, —NH—CH₂—NH₂, —CONH₂, —SO₂NH₂, —NH—CS—NH₂,

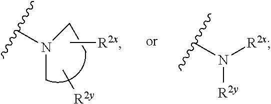

Z is a group of the formula G-L-, G-O—, G-L-O—, G-L-O-alkyl-, G-L-S—, G-SO₂—NH—, G-L-NR^{a}R^{b}-G-L-S(O)ₓ-alkyl-, G-L-CO—, G-L-aryl-, G-L-NH—CO—NH—, G-L-NH—O—, G-L-NH—NH—, G-L-NH—CS—NH, G-L-C(O)-alkyl-, G-L-SO₂—,

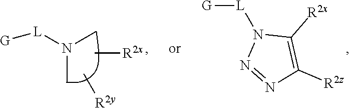

wherein:
L is a linker and G is a folate receptor binding ligand;

R^{a} and R^{b} are each, independently, H, halo, hydroxy, alkoxy, aryl, amino, acyl or C(O)R^{c}, wherein R^{c} is alkyl, aryl, oxy or alkoxy;

x is 0-3;

each of R^{2x} and R^{2y} is independently selected from a group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl;

each of R² is independently selected from a group consisting of —NH₂, —NR^{2q}R^{2q′}, —O—R^{2q};

each R^{2q} and R^{2q′} is independently alkyl or H, and

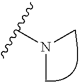 [SMILES: *N(C)C]

is a 3-10 membered N-containing non-aromatic, mono- or bicyclic heterocycle;

R²¹ is H or alkyl; and

n′ is 0-30;

wherein, in Formula II:

X¹, X², and X³ are each independently CR^{q} or N, where each R^{q} is independently hydrogen, halogen, or optionally substituted alkyl;

n is 0-30 (e.g., 1-8 or 1-6) and m is 0-4; and

wherein when n is 0, Z is not bound to Formula (II) by an oxygen atom.

One embodiment provides a compound represented by the structure of Formula (IIA):

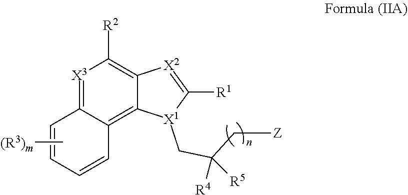

or a pharmaceutical salt thereof, wherein:
R¹ is optionally substituted alkyl (e.g., acyclic or cyclic) (e.g., optionally substituted with one or more substituent(s), each substituent independently being halogen, alkyl, heteroalkyl, alkoxy, or cycloalkyl);

R² is H, —OR^{z}, —SO₂N(R^{z})₂, —NR^{2x}R^{2y}, or N₃, where:

R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituent(s), each substituent independently being oxo, halogen, alkyl, heteroalkyl, alkoxy, or cycloalkyl); each R^{z} is independently hydrogen, halogen, or optionally substituted alkyl; or

R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);

each R³ is independently halogen, —N₃, —CN, —NO₂, alkyl, heteroalkyl, cycloalkyl, heterocycloalkyl, alkoxy, aryl, heteroaryl, heterocycloalkyl, amino, hydroxy, carboxyl, or thiol, wherein the alkyl, alkoxy, heteroalkyl, cycloalkyl, or heterocycloalkyl is optionally substituted;

R⁴ and R⁵ are each independently alkyl, alkoxy, halogen, or cycloalkyl, wherein the alkyl, alkoxy, and cycloalkyl are optionally substituted;
each X¹, X², and X³ is independently CR^{q} or N, where each R^{q} is independently hydrogen, halogen, or optionally substituted alkyl;
Z is L-G, wherein L is a linker and G is a folate receptor binding ligand;
n is 1-6; and
m is 0-4.

One embodiment provides a compound represented by the structure of Formula (III):

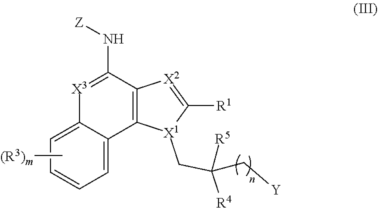

or a pharmaceutically acceptable salt thereof, wherein:
R¹, R³, R⁴, R⁵ are each independently a H, an alkyl, an alkoxyl, an alkenyl, an alkynyl, an alicyclic, an aryl, a biaryl, a halo, a heteroaryl, —COR^{2x},

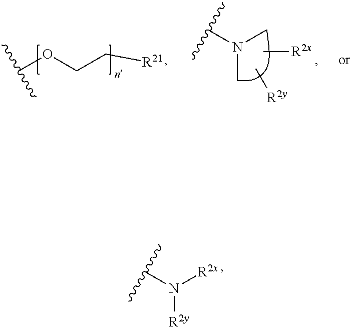

where each of R^{2x}, and R^{2y} are independently selected from a group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each of R^{2z} are independently selected from a group consisting of —NH₂, —NR^{2q}R^{2q′}, —O—R^{2q}, —SO—R^{2q}, and —COR^{2q}, wherein each R^{2q} and R^{2q′} is independently alkyl or H,

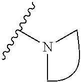 [SMILES: *N(C)C]

is a 3-10 membered N-containing non-aromatic, mono- or bicyclic heterocycle, R²¹ is H or alkyl, and n′ is 0-30;

Z is a group of the formula G-L-, G-L-CO—, G-L-C(O)-alkyl-, wherein L is a linker and G is a folate receptor binding ligand;

X¹, X², and X³ are each independently CR^{q} or N, where each R^{q} is independently hydrogen, halogen, or optionally substituted alkyl;

Y is as described in Formula I or IA;

n is 0-30; and

n is 0-4.

One embodiment provides a compound represented by the structure of Formula (IIIA):

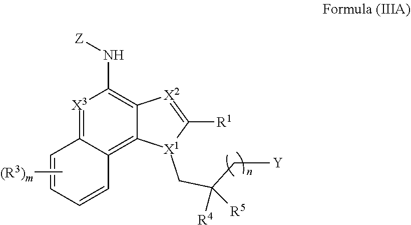

or a pharmaceutically acceptable salt thereof, wherein:
R¹ is optionally substituted alkyl (e.g., acyclic or cyclic) (e.g., optionally substituted with one or more substituent(s), each substituent independently being halogen, alkyl, heteroalkyl, alkoxy, or cycloalkyl);
Y is H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂, where:

R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituent, each substituent independently being oxo, halogen, alkyl, heteroalkyl, alkoxy, or cycloalkyl) and each R^{z} is independently hydrogen, halogen, or optionally substituted alkyl; or

R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);

each R³ is independently halogen, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;
R⁴ and R⁵ are each independently alkyl, alkoxy, halogen, or cycloalkyl, wherein the alkyl, alkoxy, and cycloalkyl are optionally substituted;
each X¹, X², and X³ is independently CR^{q} or N, where each R^{q} is independently hydrogen, halogen, or optionally substituted alkyl;
Z is L-G, wherein L is a linker and G is a folate receptor binding ligand;
n is 1-6; and
m is 0-4.

In some embodiments of Formula (II), (IIA), (III) or (IIIA), wherein X¹, X², and X³ are each N.

In some embodiments of Formula (II), (IIA), (III) or (IIIA), n is 1.

In some embodiments, the compound is represented by any one of the formulae:

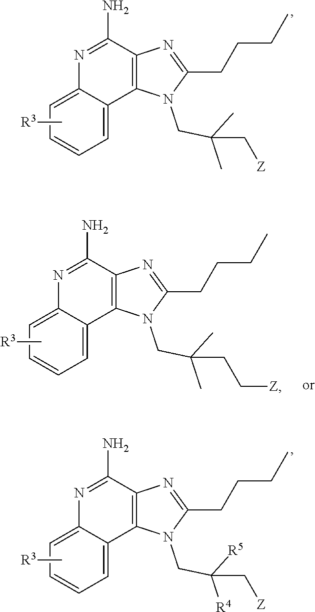

in which Z is L-G, wherein L is a linker and G is a folate receptor binding ligand, or a pharmaceutically acceptable salt thereof.

In some embodiments, the compound is a compound of Formula (IIA):

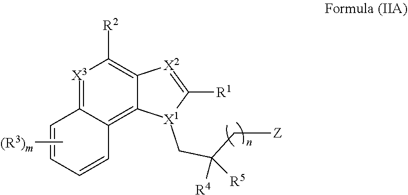

wherein:
R¹ is a C₁-C₆ alkyl optionally substituted with 1-3 substituents, each substituent independently being halogen or C₁-C₆ alkoxy;

R² is —NR^{2x}R^{2y}, where R^{2x} and R^{2y} are each independently a hydrogen or a C₁-C₆ alkyl;

each R³ is independently a halogen, —CN, C₁-C₆ alkyl, C₁-C₆ heteroalkyl, C₃-C₇ cycloalkyl, C₁-C₆ alkoxy, amino, hydroxy, carboxyl, or thiol;

R⁴ and R⁵ are each independently C₁-C₆ alkyl;

each X¹, X², and X³ is N;

Z is G-L- or G-L-O—, wherein L is a linker and G is a folate receptor binding ligand;

n is 1; and

m is 0-4;

or a pharmaceutically acceptable salt thereof.

In some embodiments, R¹ is a C₁-C₆ alkyl. In some embodiments, R² is —NH₂. In some embodiments, m is 0. In certain embodiments, R¹ is a C₁-C₆ alkyl, R² is —NH₂, n is 1, and m is 0.

In some embodiments, the compound of Formula (II) is a compound of Formula (IIB):

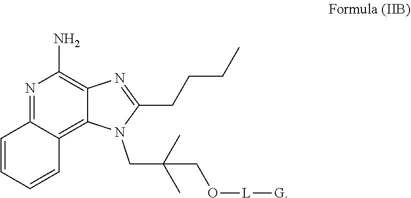

In some embodiments, the compound is represented by any one or more of the structures:

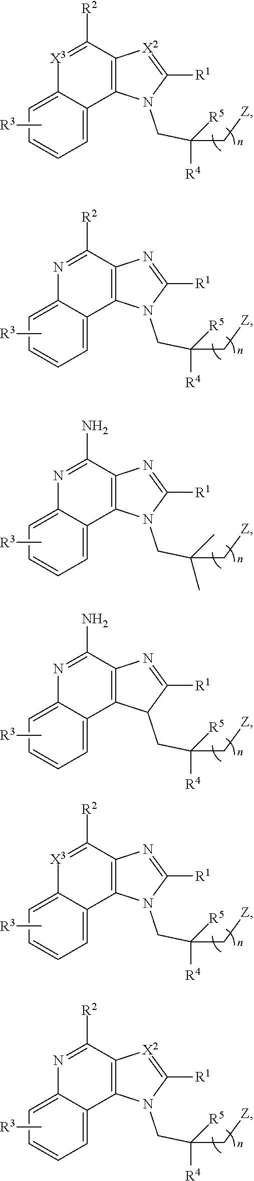

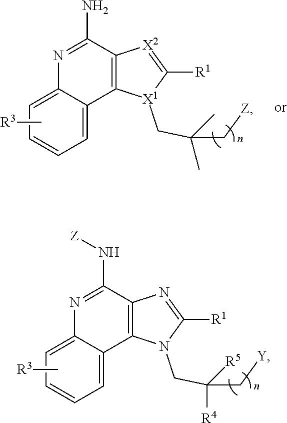

in which Z is L-G, wherein L is a linker and G is a folate receptor binding ligand, or a pharmaceutically acceptable salt thereof.

In some embodiments of Formula (II), (IIA), (IIB), (III) or (IIIA) L is a cleavable (releasable) linker. In specific embodiments of Formula (II), (IIA), (IIB), (III) or (IIIA) L is a hydrolyzable linker. In preferred embodiments, L is a non-cleavable or non-releasable linker. In specific embodiments of Formula (II), (IIA), (IIB), (III) or (IIIA) L is a non-hydrolyzable linker.

In some embodiments, L comprises an optionally substituted heteroalkyl. In some embodiments, the heteroalkyl is unsubstituted. In other embodiments, the heteroalkyl is substituted with at least one substituent selected from the group consisting of alkyl, hydroxyl, acyl, polyethylene glycol (PEG), carboxylate, and halo. In another embodiment, L comprises a substituted heteroalkyl with at least one disulfide bond in the backbone thereof.

In some embodiments, L is a peptide or a peptidoglycan with at least one disulfide bond in the backbone thereof.

In some embodiments, L is a cleavable/releasable linker that can be cleaved by enzymatic reaction, reaction oxygen species (ROS), or reductive conditions.

In some embodiments, L has the formula —NH—CH₂—CR⁶R⁷—S—S—CH₂—CH₂—O—CO—, wherein R⁶ and R⁷ are each, independently, H, alkyl, or heteroalkyl. In some preferred embodiments, L does not comprise a disulfide.

In some embodiments, L is a group or comprises a group of the formula:

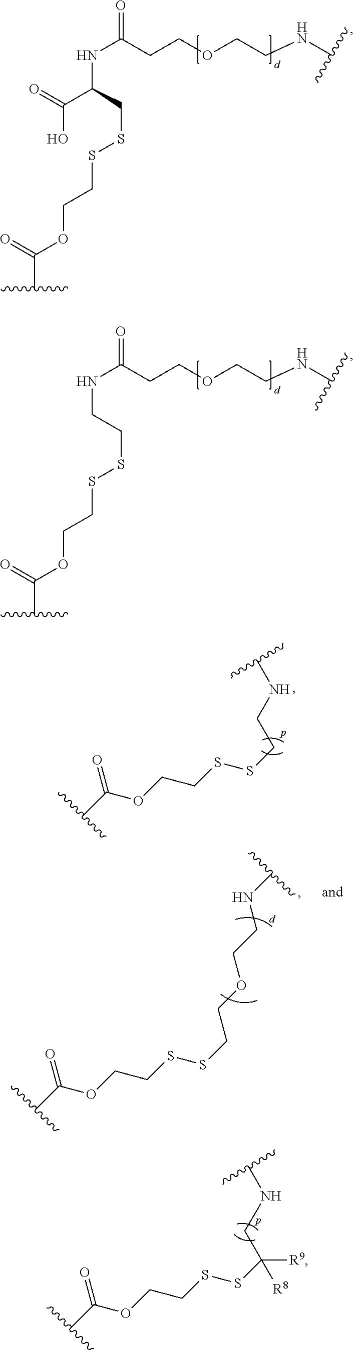

wherein p is an integer from 0 to 30; d is an integer from 1 to 40; and R⁸ and R⁹ are each, independently, H, alkyl, a heterocyclyl, a cycloalkyl, an aryl, or a heteroalkyl.

In some embodiments of Formula (II), (IIA), (IIB), (III) or (IIIA), L is a non-releasable linker. In some specific embodiments, L is a non-hydrolyzable linker.

In some embodiments, L comprises one or more group linker moieties (L′) (e.g., as expressed by L′ₙ_{^{−}}). In some embodiments, each one or more linker moieties is independently selected from the group consisting of alkylene, heteroalkylene, —O— alkynylene, alkenylene, acyl, aryl, heteroaryl, amide, oxime, ether, ester, triazole, PEG, carboxylate, carbonate, carbamate, amino acid, peptide (e.g., comprising two or more amino acid residues), and peptidoglycan.

In one embodiment, L is or comprises an alkyl ether. In another embodiment, L is or comprises an amide. In another embodiment, L is or comprises a peptide or a peptidoglycan. In another embodiment, L is or comprises an amino acid. In another embodiment, L is or comprises a PEG (e.g., —OCH₂—CH₂—O—). In another embodiment, L is or comprises polysaccharide.

In some embodiments, L is or comprises a group represented by the structure:

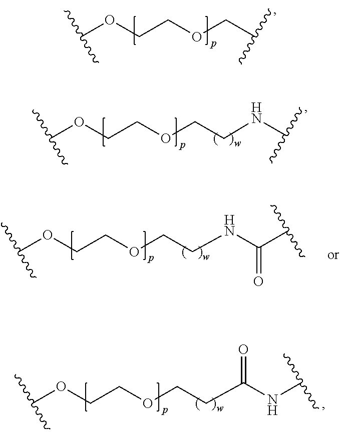

wherein w is 0-5 and p is 1-30.

In one embodiment, L is or comprises:

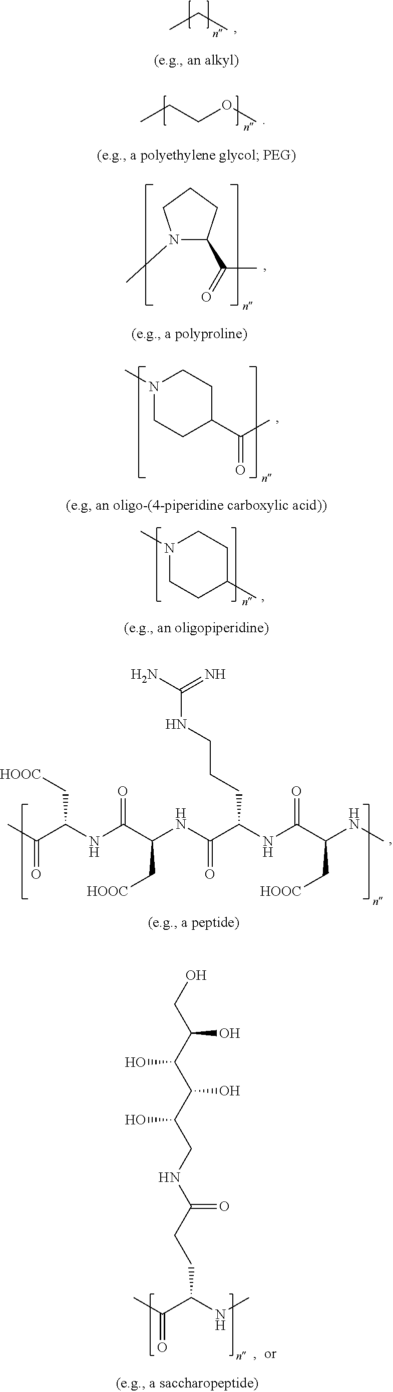

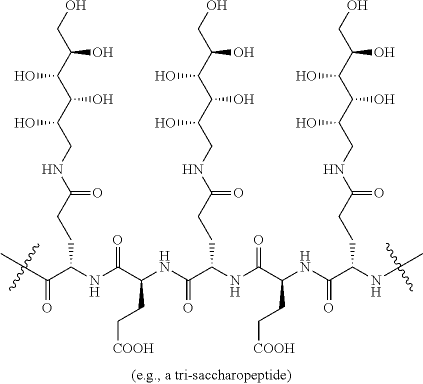

wherein n″ is an integer from 0-30 (e.g., 1-30, 1-8, or 1-6).

In certain embodiments, L is a bivalent linker.

In some embodiments of Formula (II), (IIA), (IIB), (III) or (IIIA), G is a group or comprises a group of formula (IV):

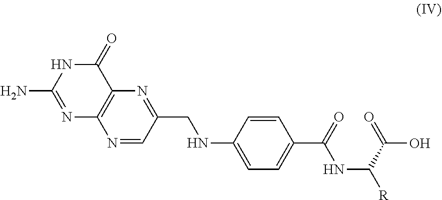

wherein R is or comprises any of the following:

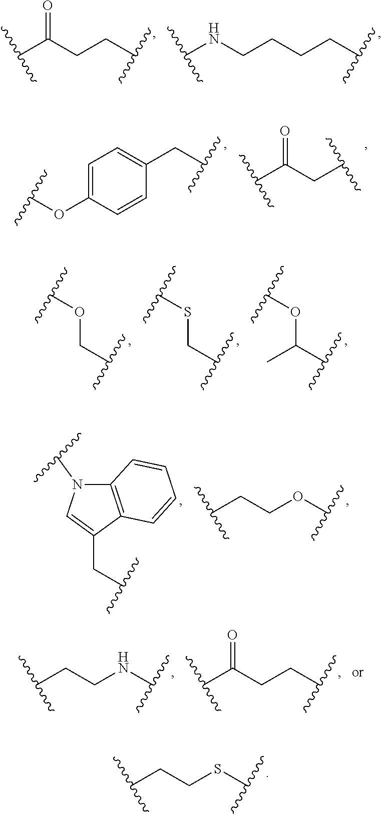

or a naturally occurring or unnatural amino acid or its derivative or fragments.

In certain specific embodiments of Formula (II), (IIA), (IIB), (III) or (IIIA), G is a radical (e.g., a group or comprising a group) having the structure of Formula (V):

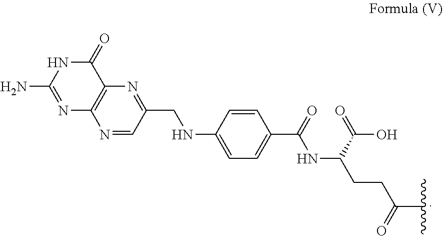

In some specific embodiments of Formula (II), (IIA), (IIB), (III) or (IIIA), G is a radical (e.g., a group or comprising a group of) having the structure of Formula (VI):

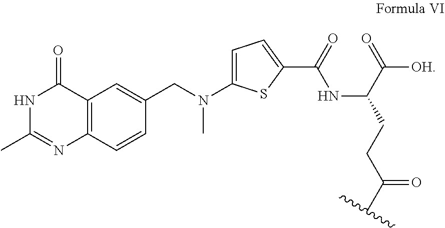 [SMILES: *C(=O)CC[C@H](NC(=O)c1ccc(N(C)Cc2ccc3nc(C)[nH]c(=O)c3c2)s1)C(=O)O]

In some embodiments, the compound is represented by one of the following structures:

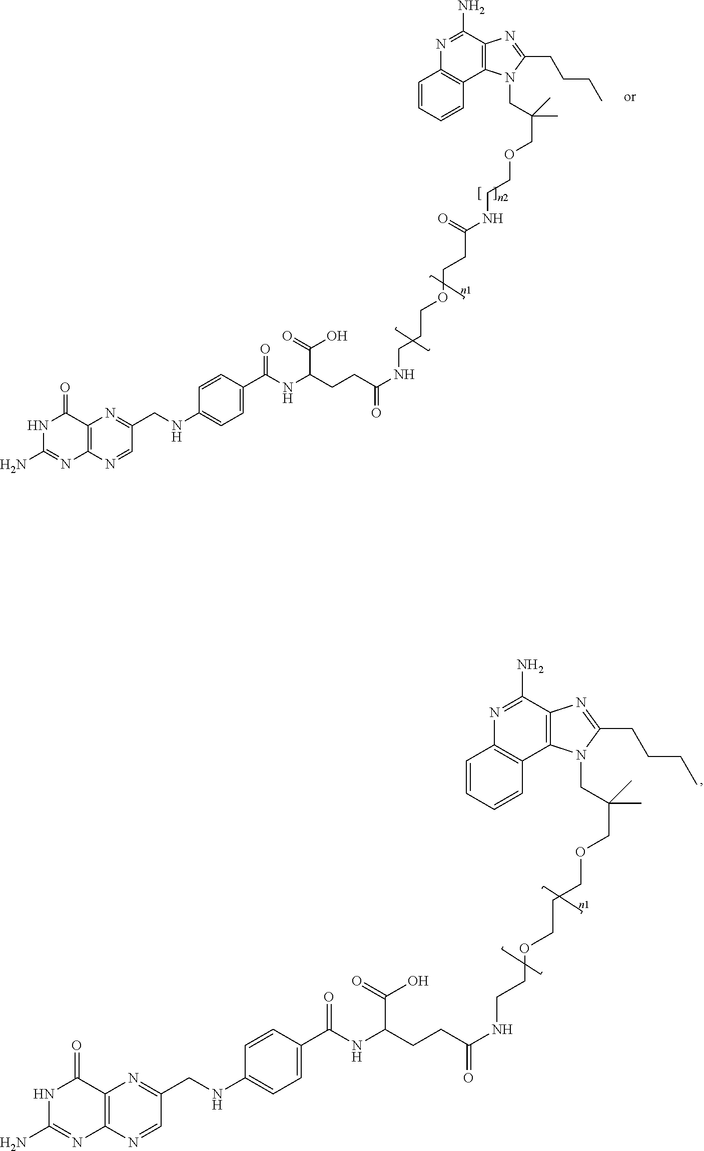

or a pharmaceutically acceptable salt thereof, wherein n1 is 0-10 and n2 is 0-10.

In some embodiments, the compound is represented by one of the following structures:

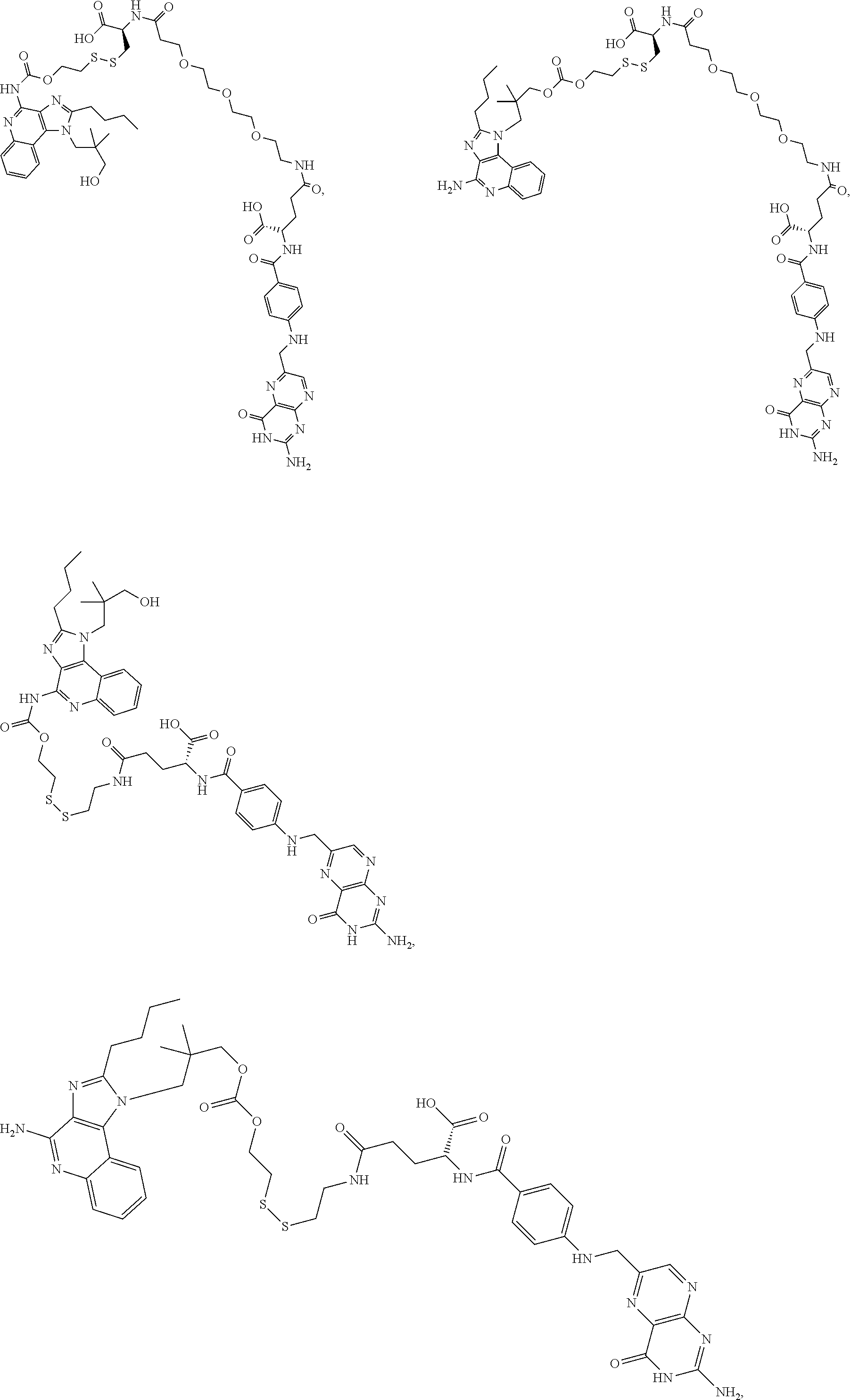

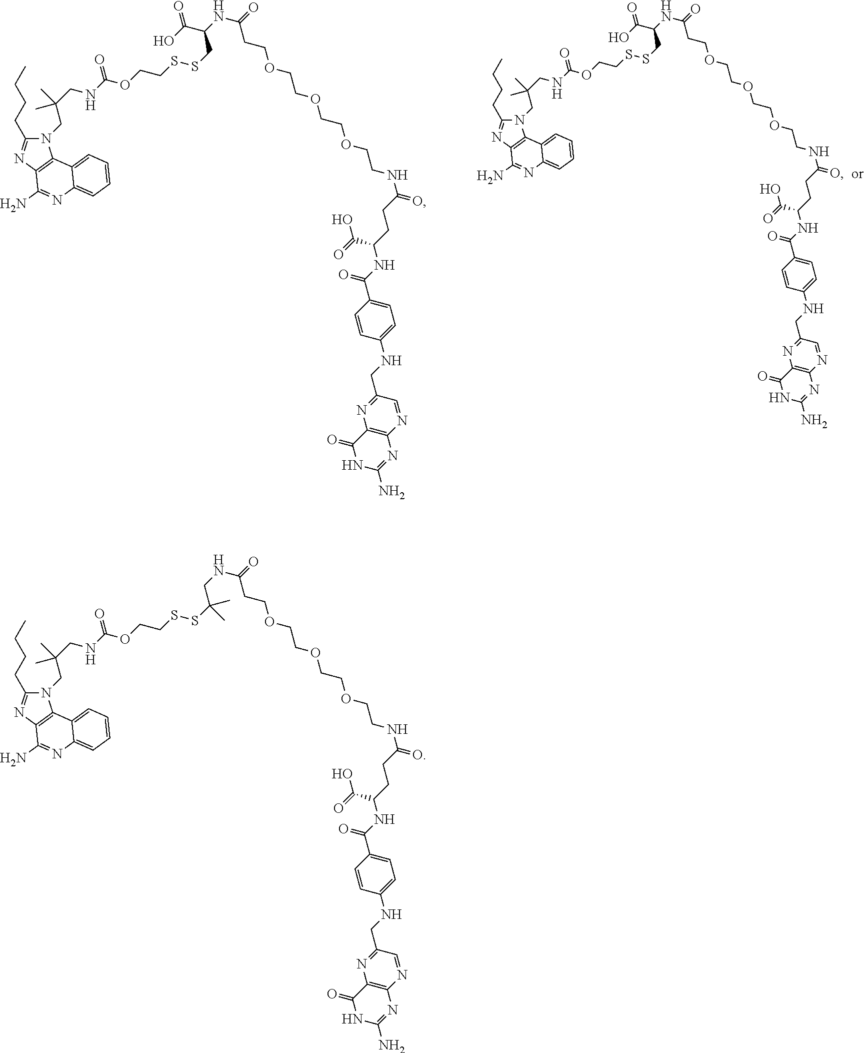

In some embodiments, the compound is represented by one of the following structures:

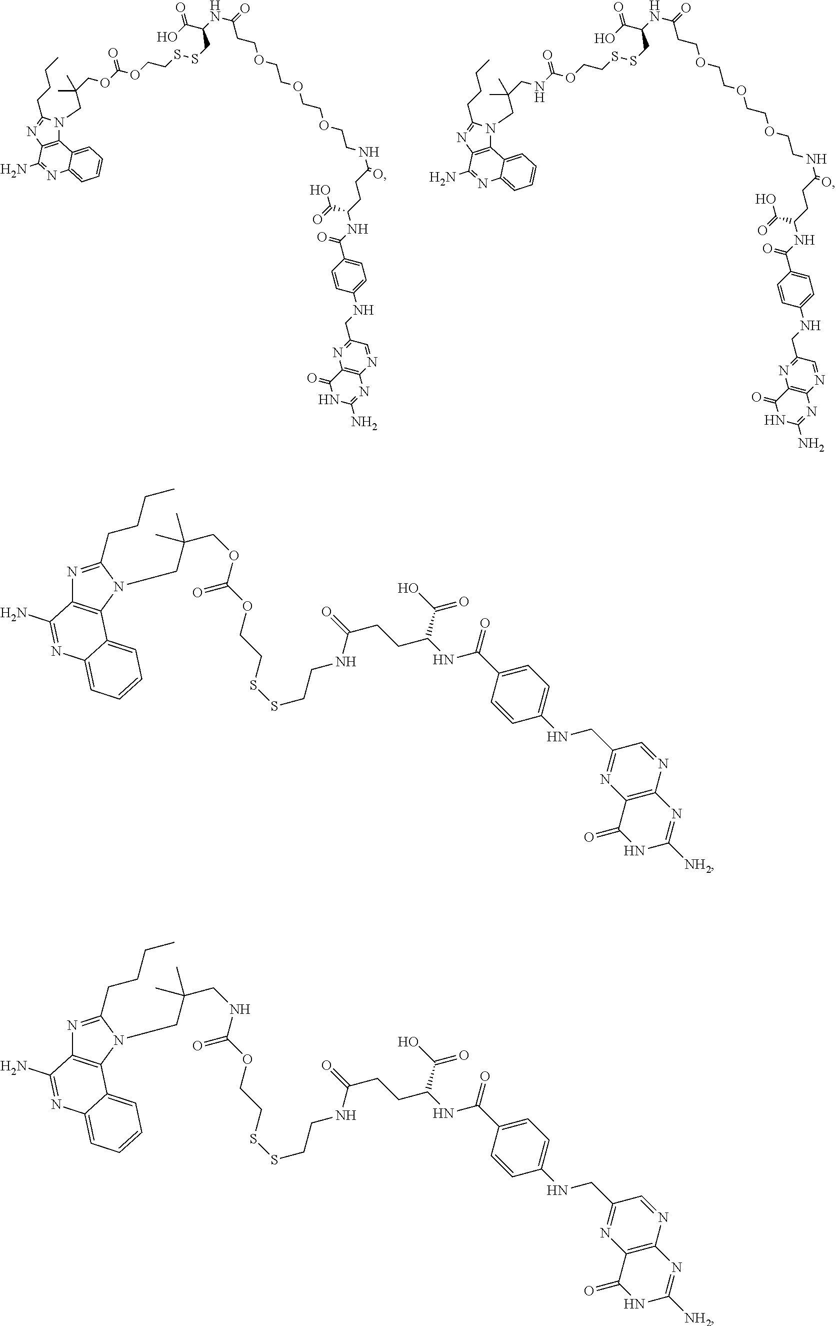

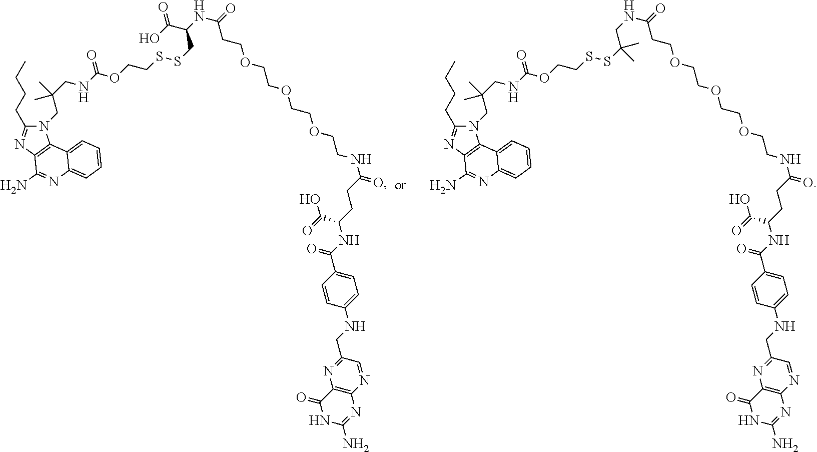

In certain embodiments, the compound is represented by one of the following structures:

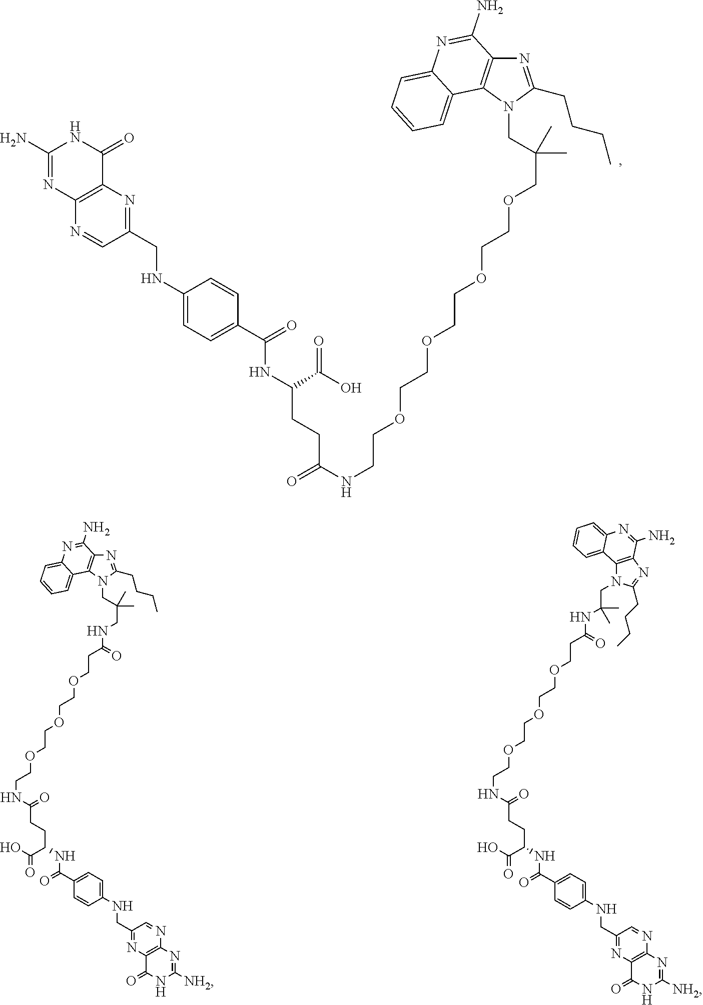

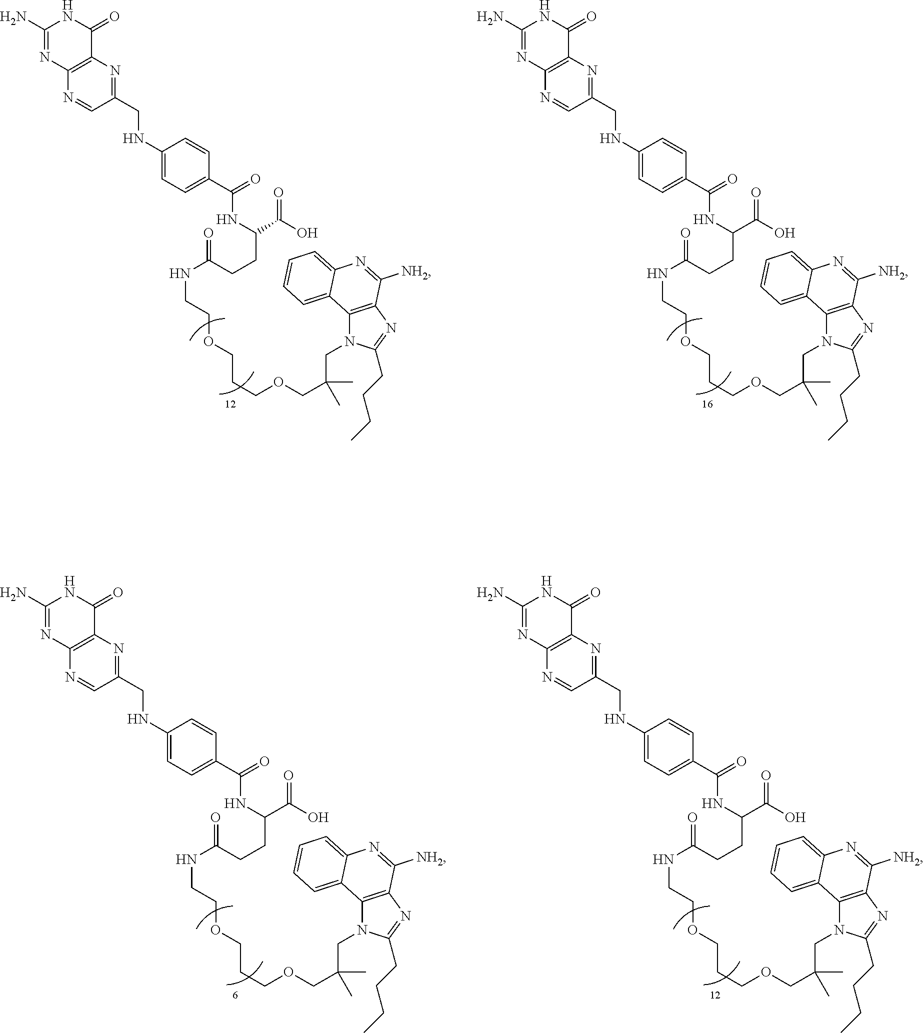

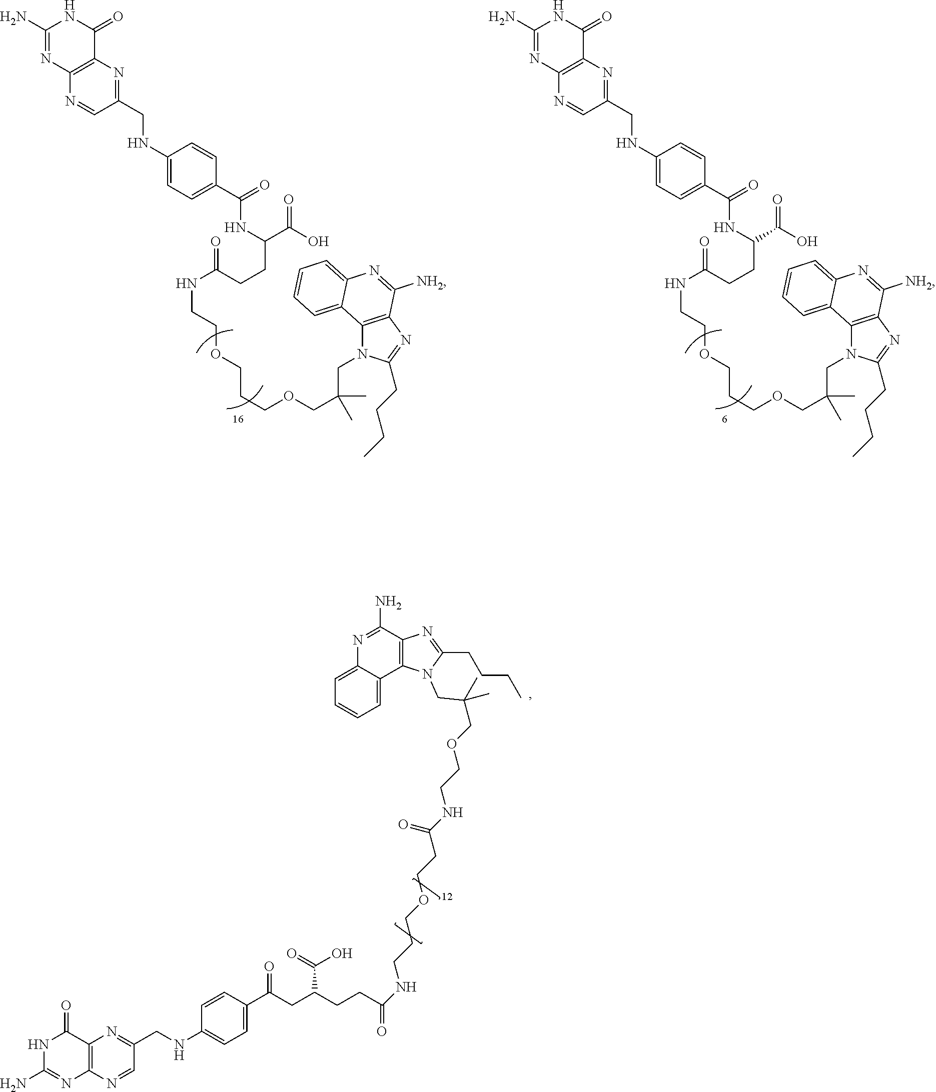

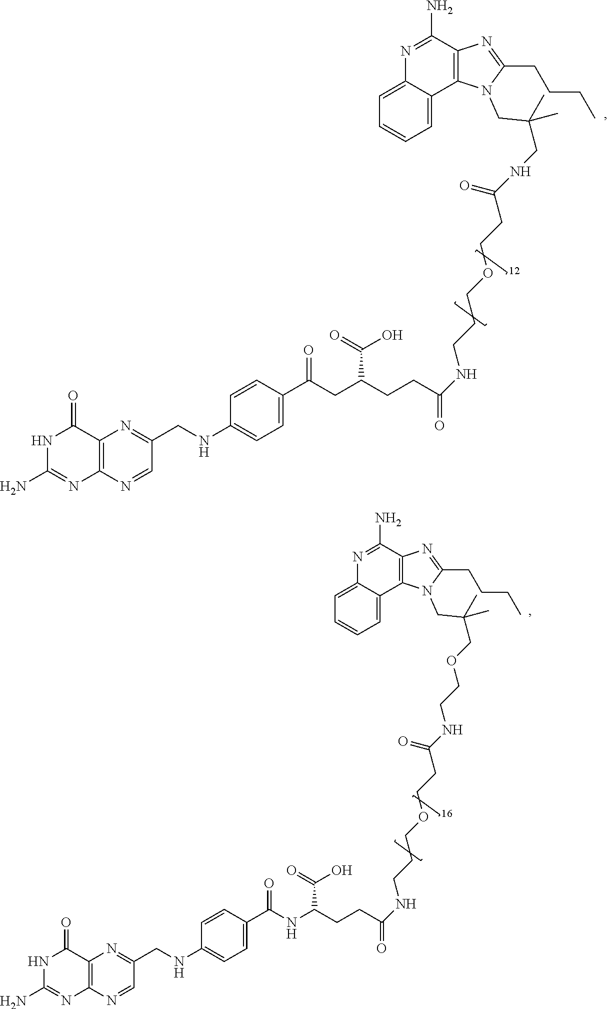

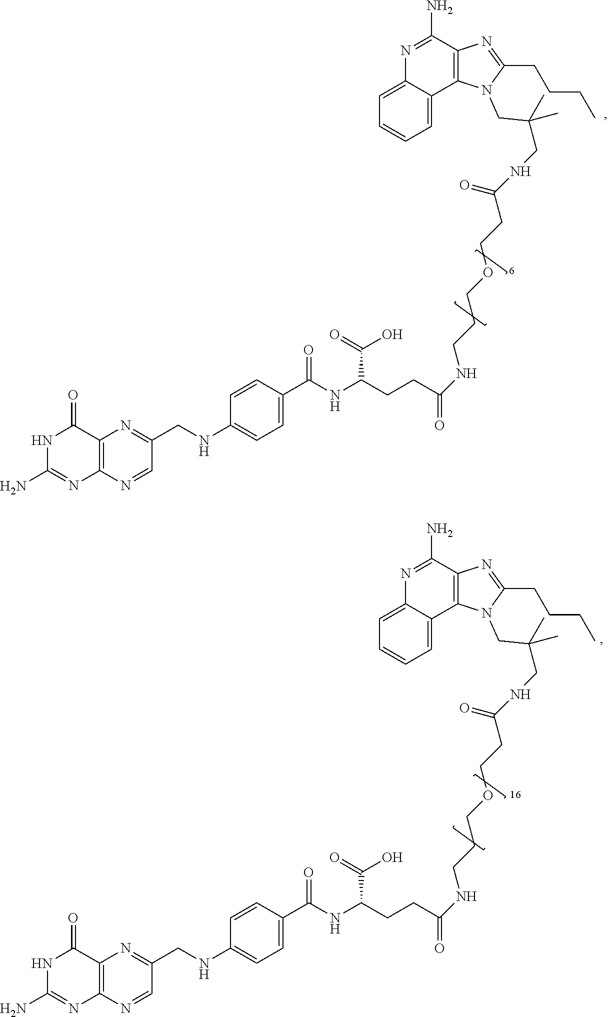

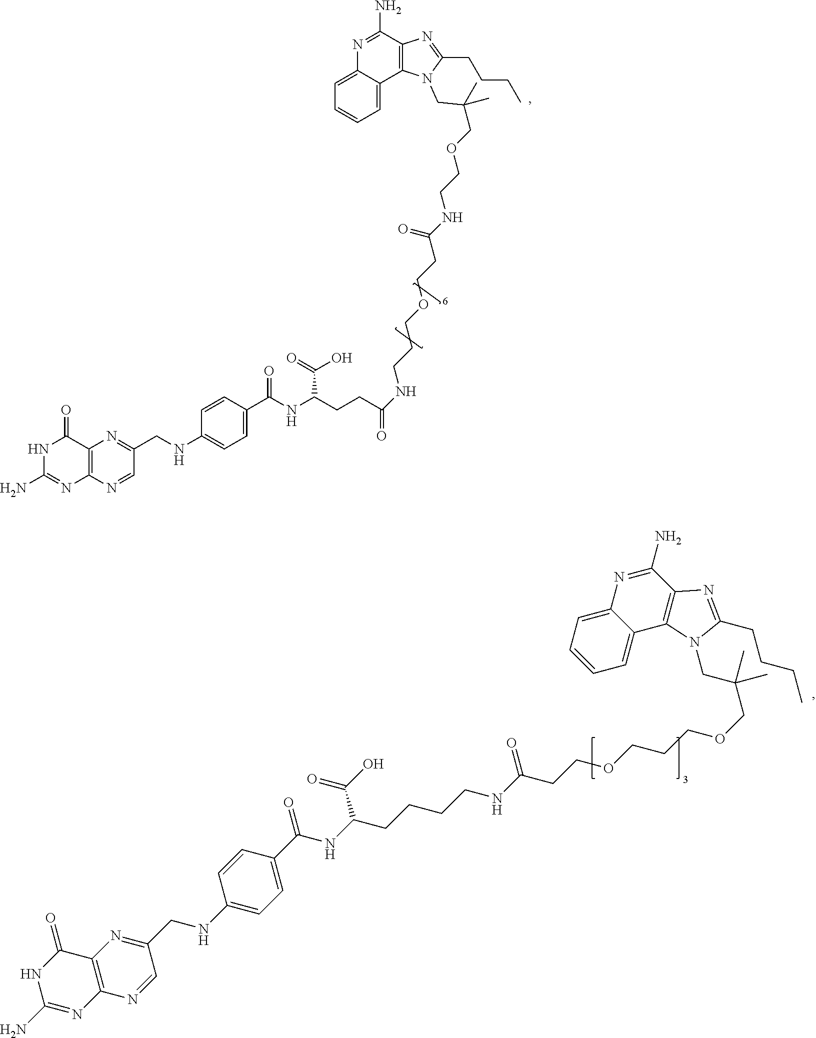

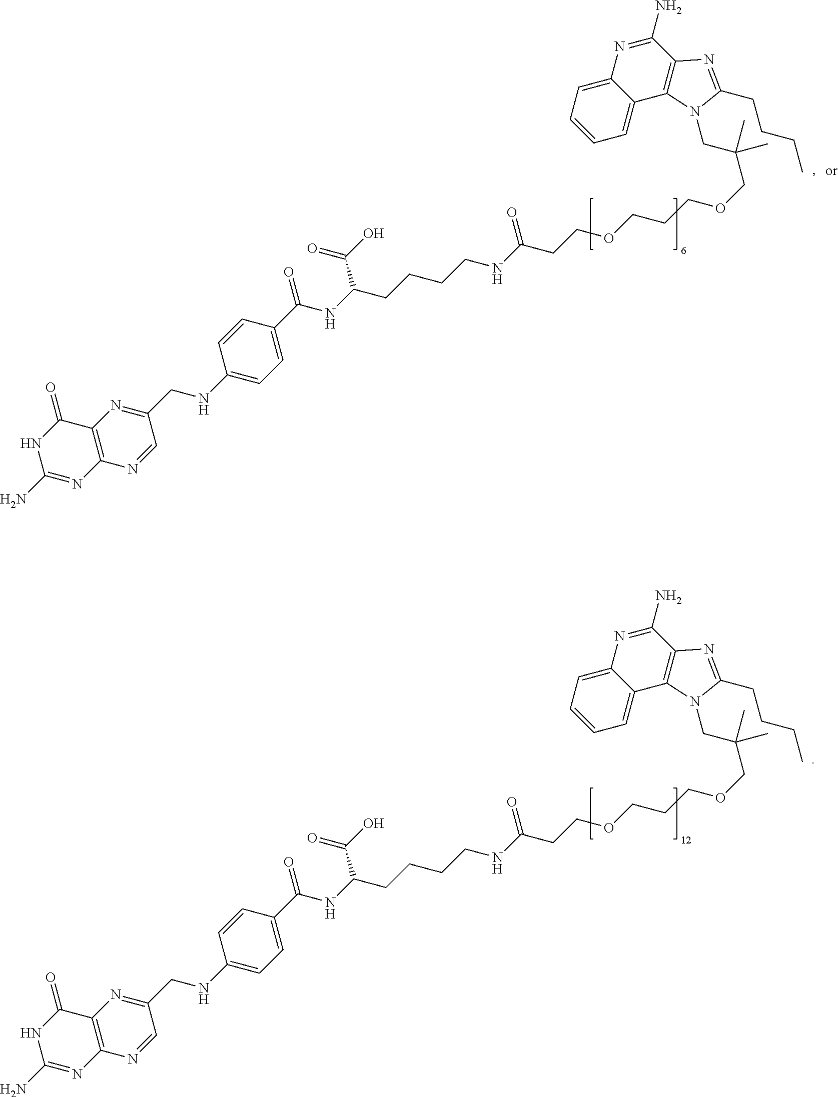

In certain embodiments, the compound is represented by one of the following structures:

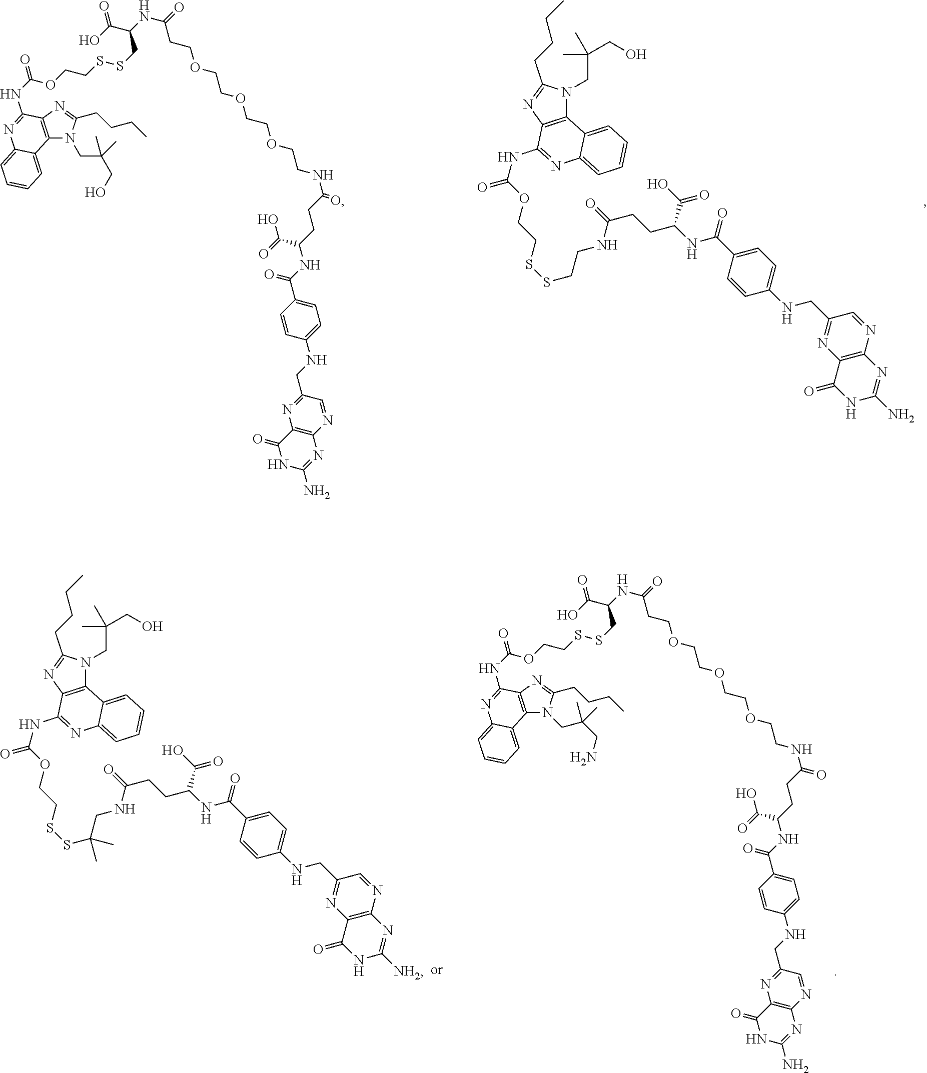

Provided is a pharmaceutical composition comprising a compound of Formula (I), (IA), (II), (IIA), (IIB), (III) or (IIIA) or any compound covered by such formulae, or a pharmaceutically acceptable salt thereof, and at least one pharmaceutically acceptable excipient.

Also provided is a pharmaceutical composition comprising a therapeutically effective compound of Formula (I), (IA), (II), (IIA), (IIB), (III) or (IIIA) or any compound covered by such formulae, or a pharmaceutically acceptable salt thereof, and at least one pharmaceutically acceptable excipient.

A method for treating a cancer or an inflammatory disease or disorder, such as, for example, a fibrotic disease or disorder, in an individual in need thereof is further provided. The method comprises administering a therapeutically effective amount of one or more compounds of any one of (I), (IA), (II), (IIA), (IIB), (III) or (IIIA) or any compound covered by such formulae, or a pharmaceutically acceptable salt thereof, or a pharmaceutical composition comprising same, to the individual or subject in need thereof.

In certain embodiments where the method is for treating an inflammatory disease or disorder, the inflammatory disease or disorder is selected from the group consisting of lupus, inflammatory bowel disease (IBS), Addison's disease, Grave's disease, Sjogren's syndrome, celiac disease, Hashimoto's thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, transplant rejection, fatty liver disease, asthma, osteoporosis, sarcoidosis, ischemia-reperfusion injury, prosthesis osteolysis, glomerulonephritis, scleroderma, psoriasis, with autoimmune myocarditis, spinal cord injury, central nervous system, viral infection, influenza, coronavirus infection, cytokine storm syndrome, bone damage, inflammatory brain disease, and atherosclerosis. In certain embodiments, the inflammatory disease or disorder is a fibrotic disease or disorder.

A method for treating a fibrotic disease or disorder in an individual in need thereof is even still further provided. The method comprises administering a therapeutically effective amount of one or more compounds of any one of (I), (IA), (II), (IIA), (IIB), (III) or (IIIA) or any compound covered by such formulae, or a pharmaceutically acceptable salt thereof, or a pharmaceutical composition comprising same, to an individual or subject in need thereof. In certain embodiments, the fibrotic disease or disorder is selected from the group consisting of arthrofibrosis, autoimmune pancreatitis, bladder fibrosis, chronic kidney disease, chronic wounds, Crohn's disease, desmoid tumor, Dupuytren's contracture, endometrial fibroids, fibromatosis, graft-versus-host disease (GVHD), heart fibrosis, keloids, liver fibrosis (e.g., nonalcoholic steatohepatitis (NASH) or cirrhosis), mediastinal fibrosis, myelofibrosis, nephrogenic systemic fibrosis, Peyronie's disease, pulmonary fibrosis, retroperitoneal cavity fibrosis, scleroderma or systemic sclerosis, and skin fibrosis.

Another method provided is one for treating a cancer in an individual in need thereof. The method comprises administering (e.g., to the individual) a compound of Formula (I), (IA), (II), (IIA), (IIB), (III) or (IIIA) or any compound covered by such formulae, or a pharmaceutically acceptable salt thereof, or a pharmaceutical composition comprising same, to an individual in need thereof. In one embodiment, the cancer is selected from the group consisting of lung cancer, bone cancer, pancreatic cancer, skin cancer, cancer of the head, cancer of the neck, cutaneous melanoma, intraocular melanoma, uterine cancer, ovarian cancer, endometrial cancer, epithelial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, bladder cancer, gastric cancer, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer, and adenocarcinoma of the gastroesophageal junction. In additional embodiments, the cancer is lung cancer, breast cancer (e.g., triple negative breast cancer), colon cancer, gastric cancer, bladder cancer, ovarian cancer, pancreatic cancer, or epithelial cancer.

Yet another method provided is one for inhibiting or reducing fibrosis (e.g., in an individual in need thereof, such as an individual suffering from cancer or fibrotic disease). The method comprises administering (e.g., to the individual) a compound of Formula (I), (IA), (II), (IIA), (IIB), (III) or (IIIA) or any compound covered by such formulae, or a pharmaceutically acceptable salt thereof, or a pharmaceutical composition comprising same, to an individual in need thereof. In some embodiments, the fibrotic disease or disorder is selected from the group consisting of arthrofibrosis, autoimmune pancreatitis, bladder fibrosis, chronic kidney disease, chronic wounds, Crohns's disease, desmoid tumor, Dupuytren's contracture, endometrial fibroids, fibromatosis, graft-versus-host disease, heart fibrosis, keloids, liver fibrosis (e.g., NASH or cirrhosis), mediastinal fibrosis, myelofibrosis, nephrogenic systemic fibrosis, Peyronie's disease, pulmonary fibrosis, retroperitoneal cavity fibrosis, scleroderma or systemic sclerosis, and skin fibrosis.

In one embodiment, the fibrotic disease or disorder is idiopathic pulmonary fibrosis, liver fibrosis, myelofibrosis, or cardiac fibrosis. In certain embodiments, the fibrotic disease or disorder is pulmonary fibrosis, liver fibrosis, scleroderma, myelofibrosis, Crohn's disease, or chronic kidney disease.

A method for inhibiting or reducing fibrosis (e.g., in an individual in need thereof, such as an individual suffering from cancer or fibrotic disease) is provided, such method comprising administering (e.g., to the individual) a compound of Formula (I), (IA), (II), (IIA), (IIB), (III) or (IIIA) or any compound covered by such formulae, or a pharmaceutically acceptable salt thereof, or a pharmaceutical composition comprising the same, to an individual in need thereof in an amount effective to convert a population of macrophages biased towards an M2-like phenotype (e.g., profibrotic) to an M1-like phenotype (e.g., antifibrotic), wherein the population of macrophages are present in a targeted location within the individual, the M2-like phenotype is associated with an anti-inflammatory/pro-fibrotic state, and the M1-like phenotype is associated with a proinflammatory/anti-fibrotic state.

In addition, a method for inhibiting or reducing cancerous growth (e.g., in an individual in need thereof, such as an individual suffering from cancer) is provided, such method comprising administering (e.g., to the individual) a compound of Formula (I), (IA), (II), (IIA), (IIB), (III) or (IIIA) or any compound covered by such formulae, or a pharmaceutically acceptable salt thereof, or a pharmaceutical composition comprising the same, to an individual in need thereof in an amount effective to convert a population of macrophages biased towards an M2-like phenotype (e.g., profibrotic) to an M1-like phenotype (e.g., antifibrotic), wherein the population of macrophages are present in a targeted location within the individual, the M2-like phenotype is associated with an anti-inflammatory/pro-fibrotic state, and the M1-like phenotype is associated with a proinflammatory/anti-fibrotic state. In at least one embodiment, the targeted location is a tumor microenvironment.

In any of the above embodiments, the method does not induce unwanted inflammation in the individual.

In any of the above embodiments, the method further comprises administering a second therapeutic agent. In one embodiment, the second therapeutic agent is an anti-inflammatory agent. In any of the above embodiments, the method further comprises administering a chemotherapeutic agent.

Further embodiments and the full scope of applicability of the present disclosure will become apparent from the Detailed Description. However, it should be understood that the Detailed Description and specific examples are given by way of illustration only. Various changes and modifications within the spirit and scope of the present disclosure will become apparent to those skilled in the art.

### DESCRIPTION OF THE DRAWINGS

The detailed description will be better understood when taken in conjunction with the accompanying drawings, wherein:

FIG. 1 shows the chemical structure of exemplary compounds provided herein.

FIG. 2 shows the effect of various exemplary compounds on interleukin-6 (IL-6) expression in peripheral blood mononuclear cells (PBMCs).

FIG. 3A and FIG. 3B show the in vitro effects of various exemplary compounds on IL-6 (FIG. 3A) and CXCL-10 (FIG. 3 B) induction in human monocyte-derived M2-macrophages for 48 hours.

FIG. 3C and FIG. 3D show the in vivo effects in mice of various exemplary compounds on IL-6 (FIG. 3C) and tumor necrosis factor alpha (TNF-α) (FIG. 3D) production.

FIG. 4A and FIG. 4B show the effects of Toll-like receptor 7 (TLR7) agonists on IL-6 and TNF-α in mouse bone marrow-derived M2-macrophages.

FIG. 5A and FIG. 5B show flow cytometry data of expression of TLR7 and folate receptor β (FR-β) in fixed and permeabilized human M2-polarized macrophages.

FIG. 6 show confocal microscopic images of human PBMC-derived M2 macrophages fixed, permeabilized, and stained with both TLR7 antibody and FR-β antibody (results showing that both are located within the endosome).

FIG. 7A and FIG. 7B show liquid chromatography mass spectrometry (LCMS) data of a disulfide cleavage study of folate (FA)-TLR7 conjugates FA-TLR7-1 (FIG. 7A) and FA-Compound 1 (releasable conjugate) (Compound 5) (FIG. 7B), with analysis performed at 0 minutes, 7 minutes, 30 minutes and 50 minutes.

FIG. 8 shows a schematic diagram of what are believed to be examples of possible mechanisms of action of certain releasable and non-releasable folate-TLR7 conjugates based on data obtained to date.

FIG. 9 shows graphical data representative of M1 marker IL-6 expression in PBMC-derived macrophages following treatment with a releasable folate-TLR7 conjugate (FA-PEG₃-TLR7-1A (Releasable (“Re”))).

FIGS. 10A-10E show graphical data related to an in vivo therapeutic study of a releasable folate-TLR7 conjugate (Compound 5; FA-PEG₃-TLR7-1A (Re)) (10 nmol/mice) in a 4T1 solid tumor model. FIG. 10A shows tumor volume measured every other day after the treatment. FIG. 10B shows average tumor weight as measured at the end of the study. FIG. 10C shows the relative ratio of M1/M2 (CD86+/CD206+) macrophages. FIG. 10D shows CD8+ cells per 100,000 events. FIG. 10E shows CD4+ cells per 100,000 events.

FIGS. 11A-11D show graphical data related to various M1 markers in human PBMC-derived macrophages following treatment with a non-releasable folate-TLR7 conjugate of the present disclosure (Compound 4; FA-PEG₃-TLR7-1A (NR)). FIG. 11A shows changes in mRNA levels of the M1 marker IL-6 after 3 hrs of treatment, whereas FIG. 11B shows changes in mRNA levels of the M1 marker TNF-α after 3 hours of treatment. FIG. 11C shows analysis of IL-6 protein expression using ELISA, after 3+45 hours of treatment. FIG. 11D shows M2 marker CD206 surface expression of macrophages analyzed by flow cytometry.

FIG. 12A and FIG. 12B show graphical data related to cell-surface markers CD40 and CD80, respectively, analyzed using flow cytometry, of human PBMC-derived M2 macrophages following treatment with a non-releasable folate-TLR7 conjugate (Compound 4; FA-PEG₃-TLR7-1A (NR)).

FIG. 13 shows in vivo pharmacokinetic analysis data of a non-releasable folate-TLR7 conjugate (Compound 4; FA-PEG₃-TLR7-1A (NR)) in mice (column: Agilent Eclipse Plus C18, 2.1×50 mm, SN: B17477, Eluent: A—water+0.1% FA, B—CAN+0.1% FA).

FIG. 14A and FIG. 14B show graphical data related to an in vivo therapeutic study of a non-releasable folate-TLR7 conjugate (Compound 4; FA-PEG₃-TLR7-1A (NR)) at different concentrations and different dosing intervals in a 4T1 solid tumor model. FIG. 14A shows tumor volume measured after the treatment. FIG. 14B shows tumor weight (grams) measured at the end of the study.

FIGS. 15A-15C show data from an in vivo therapeutic study of a non-releasable folate-TLR7 conjugate (Compound 4; FA-PEG₃-TLR7-1A (NR)) (1 nmol once/week) in a 4T1 metastatic tumor model. FIG. 15A shows tumor volume measured during the treatment, whereas FIG. 15B shows the quantification of metastatic tumor cells in the lung using a 6-thioguanine assay, and FIG. 15C shows representative images from the 6-thioguanine assay of FIG. 15B.

### DETAILED DESCRIPTION

The present disclosure relates to the preparation and use of compounds and compositions that prevent and/or treat fibrotic diseases. In certain embodiments, the compounds, compositions, and methods provided are also useful for the prevention and/or treatment of cancer. In some embodiments, the compounds, compositions, and methods provided leverage strategies to (e.g., selectively) target the innate immune system and reprogram the polarization of a macrophage from M2 to M1 and, for example, leverage the anti-fibrotic/pro-inflammatory properties of the M1-type phenotype.

In at least one embodiment, such compounds and compositions comprise an immune modulator or a pharmaceutically acceptable salt thereof (e.g., a Toll-like receptor (TLR) agonist such as a TLR7 or TLR7/8 agonist) that can, upon administration, convert—e.g., reprogram—activated myeloid cells (e.g., M2-like macrophages) into an anti-fibrotic/pro-inflammatory M1 polarization.

In exemplary embodiments, the immune modulator or pharmaceutically acceptable salt thereof is conjugated (directly or via a linker) to a targeting moiety or radical thereof that targets a pattern recognition receptor of a fibrotic or cancerous cell. The targeting moiety or radical thereof, for example, can be a folate ligand or functional fragment or analog thereof. Such embodiments utilize the limited expression of the targeting moiety's target to localize systemically administered compounds directly to the target-expressing cells (e.g., those of fibrotic and/or cancerous tissue) such that the immune modulator can then reprogram the activated myeloid cells (e.g., M2-like macrophages) into an antifibrotic/proinflammatory M1 polarization. This targeting design advantageously can prevent the systemic activation of the immune system and, thus, avoid toxicity in the subject.

Further, exemplary embodiments can comprise a linker disposed between the targeting moiety and the immune modulator or pharmaceutically acceptable salt thereof. Such linkers can be releasable or non-releasable. A compound that comprises a releasable linker (as well as a composition comprising the compound) will, when administered, result in the targeting moiety and immune modulator being released from each other on or about the time the immune modulator becomes active. In embodiments where a compound comprises a non-releasable linker (as well as a composition comprising the compound) is administered, the targeting moiety and the immune modulator do not release quickly under physiological conditions. In this way, the components remain together following uptake by a targeted cell (e.g., a fibrotic or cancerous cell) and/or activation of the immune modulator.

Various embodiments will now be described, as well as data relating to examples that support the same.

### Compounds

One embodiment provides a compound (e.g., an immune modulator) represented by the structure of Formula (I):

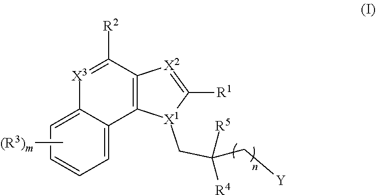

or a pharmaceutically acceptable salt thereof, wherein:

R¹, R³, R⁴, R⁵ are each independently a H, an alkyl, an alkoxyl, an alkenyl, an alkynyl, a cycloalkyl, an alicyclic, an aryl (e.g., a biaryl), a halo, a heteroaryl, —COR^{2x},

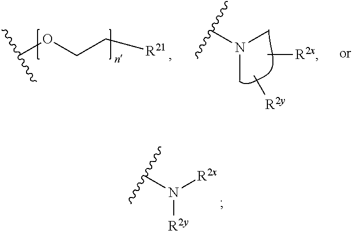

R² is a H, —OH, —NH₂, —NHR^{2x}, N₃, —NH—CH₂—NH₂, —CONH₂, —SO₂NH₂, —NH—CS—NH₂,

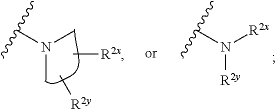

Y is a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

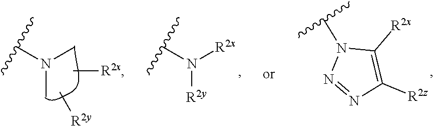

where each of R^{2x}, and R^{2y} is independently selected from a group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl,
each of R^{2z} are independently selected from a group consisting of —NH₂, —NR^{2q}R^{2q′}, —O—R^{2q}, —SO—R^{2q}, and —COR^{2q}; wherein each R^{2q} and R^{2q′} is independently alkyl or H, and

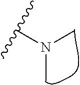 [SMILES: *N(C)C]

is a 3-10 membered N-containing non-aromatic, mono- or bicyclic heterocycle;

R²¹ is H or alkyl; and

n′ is 0-30;

where, in Formula (I), each of X¹, X², X³ is independently CR^{q} or N, where each R^{q} is independently hydrogen, halogen, or optionally substituted alkyl;

n is 0-30; and

m is 0-4.

In at least one embodiment of Formula (I), when n is 0, Y is not H, —OH, or —O—R^{2x}.

Another embodiment provides a compound (e.g., an immune modulator), having the structure of Formula (IA):

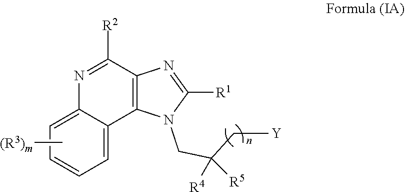

or a pharmaceutically acceptable salt thereof, wherein:

R¹ is an optionally substituted C₃-C₈ alkyl (e.g., acyclic or cyclic) (e.g., optionally substituted with one or more substituent(s), each substituent independently being halogen, alkyl, heteroalkyl, alkoxy, or cycloalkyl);

R² is H, —OR^{z}, —SO₂N(R^{z})₂, —NR^{2x}R^{2y}, or N₃;

Y is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, where:
R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituent(s), each substituent independently being oxo, halogen, alkyl, heteroalkyl, alkoxy, or cycloalkyl), where each R^{z} is independently hydrogen, halogen, or optionally substituted alkyl; or
R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);

each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein the alkyl, alkoxy, heteroalkyl, cycloalkyl, or heterocycloalkyl is optionally substituted;

R⁴ and R⁵ are each independently alkyl, alkoxy, halogen, or cycloalkyl, wherein the alkyl, alkoxy, and cycloalkyl are optionally substituted;

n is 1-6; and

m is 0-4.

One embodiment provides a compound, or a pharmaceutically acceptable salt thereof, having the structure of Formula (I) or (IA) wherein n is 1-30. In one embodiment, n is 1-6. In another embodiment, n is 1-3. In another embodiment, n is 1 or 2. In another embodiment, n is 0. In another embodiment, n is 1. In another embodiment, n is 1 and Y is OH. In another embodiment, n is 1 and Y is NH₂. In one embodiment, the compound is represented by the structure of Compound 1:

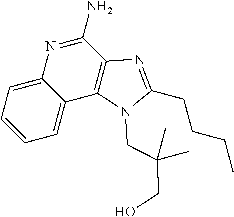 [SMILES: CCCCc1nc2c(N)nc3ccccc3c2n1CC(C)(C)CO]

or a pharmaceutically acceptable salt thereof.

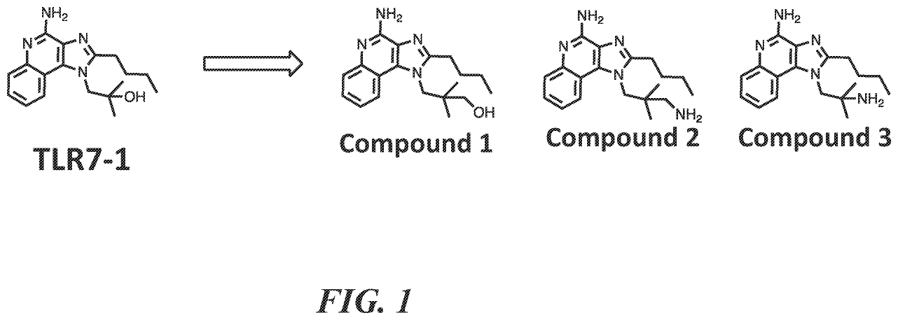

In one embodiment, the compound is represented by the structure of Compound 2. In one embodiment, the compound is represented by the structure of Compound 3. The structures of such compounds are depicted in FIG. 1.

One embodiment provides a compound, or a pharmaceutically acceptable salt thereof, having the structure of Formula (I) or (IA) wherein Y is —OH, OCH₃, —NH₂, —NHNH₂, —NHCONH₂, —SH, —SO₂NH₂, —N₃, —COOH, —COCH₃, —COOCH₃, or —CONH₂.

One embodiment provides a compound, or a pharmaceutically acceptable salt thereof, having the structure of Formula (I) or (IA) wherein Y is a H, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

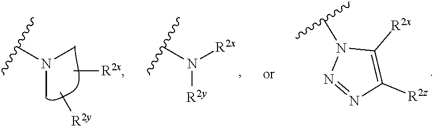

One embodiment provides a compound, or a pharmaceutically acceptable salt thereof, having the structure of Formula (I) or (IA) wherein Y is OH.

One embodiment provides a compound, or a pharmaceutically acceptable salt thereof, having the structure of Formula (I) or (IA) wherein Y is NH₂.

One embodiment provides a compound, or a pharmaceutically acceptable salt thereof, having the structure of Formula (I) or (IA) wherein n is 1 and Y is OH.

One embodiment provides a compound, or a pharmaceutically acceptable salt thereof, having the structure of Formula (I) or (IA) wherein n is 1 and Y is NH₂.

One embodiment provides a compound, or a pharmaceutically acceptable salt thereof, having the structure of Formula (I) or (IA) wherein n is 0 and Y is NH₂.

One embodiment provides a compound, or a pharmaceutically acceptable salt thereof, having the structure of Formula (I) or (IA) wherein R¹ is an optionally substituted alkyl. In one embodiment, R¹ is an optionally substituted C₃-C₆ alkyl. In another embodiment, R¹ is an optionally substituted acyclic C₃-C₆ alkyl. In another embodiment, R¹ is butyl.

One embodiment provides a compound, or a pharmaceutically acceptable salt thereof, having the structure of Formula (I) or (IA) wherein R² is —NR^{2x}R^{2y}. In one embodiment, R² is NH₂.

One embodiment provides a compound, or a pharmaceutically acceptable salt thereof, having the structure of Formula (I) or (IA) wherein R³ is H.

One embodiment provides a compound, or a pharmaceutically acceptable salt thereof, having the structure of Formula (I) or (IA) wherein R⁴ is alkyl. In one embodiment, R⁴ is methyl.

One embodiment provides a compound, or a pharmaceutically acceptable salt thereof, having the structure of Formula (I) or (IA) wherein R⁵ is alkyl. In one embodiment, R⁵ is methyl.

One embodiment provides a compound, or a pharmaceutically acceptable salt thereof, having the structure of Formula (I) or (IA) wherein R⁴ and R⁵ are each alkyl. In one embodiment, R⁴ and R⁵ are each independently C₁-C₄ alkyl. In one embodiment, R⁴ and R⁵ are each methyl.

One embodiment provides a compound, or a pharmaceutically acceptable salt thereof, having the structure of Formula (I) or (IA) wherein m is 0. In another embodiment, m is 1. In another embodiment, m is 2. In another embodiment, m is 3. In another embodiment, m is 4.

One embodiment provides a compound, or a pharmaceutically acceptable salt thereof, having the structure of Formula (I) wherein X¹, X², and X³ are each N. In one embodiment, X¹ is N. In another embodiment, X² is N. In another embodiment, X³ is N.

One embodiment provides a compound, or a pharmaceutically acceptable salt thereof, having the structure of Formula (I) with the proviso that compounds in which n is 0 are excluded.

One embodiment provides a compound, or a pharmaceutically acceptable salt thereof, having the structure of Formula (I) with the proviso that compounds in which n is 0 and Y is OH are excluded.

One embodiment provides a compound, or a pharmaceutically acceptable salt thereof, having the structure of Formula (I) with the proviso that compounds wherein n is 0, Y is OH, R¹ is butyl, R² is NH₂, R³ is H and R⁴ and R⁵ are each methyl are excluded.

One embodiment provides a compound, or a pharmaceutically acceptable salt thereof, having the structure of Formula (I) with the proviso that the compound TLR7-1 as shown in FIG. 1 is excluded.

In some embodiments, the compound is represented by any one or more of the formulae:

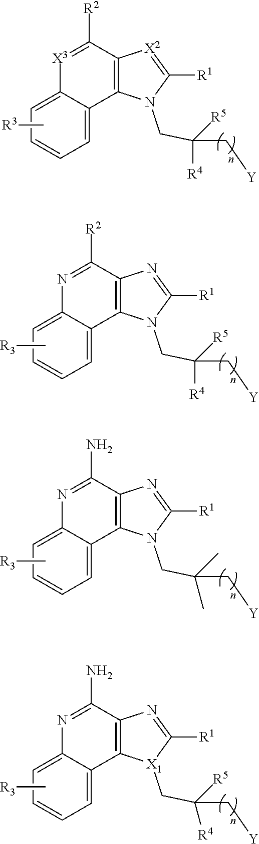

or a pharmaceutically acceptable salt thereof.

In some embodiments, the compound is represented by any one or more of the formulae:

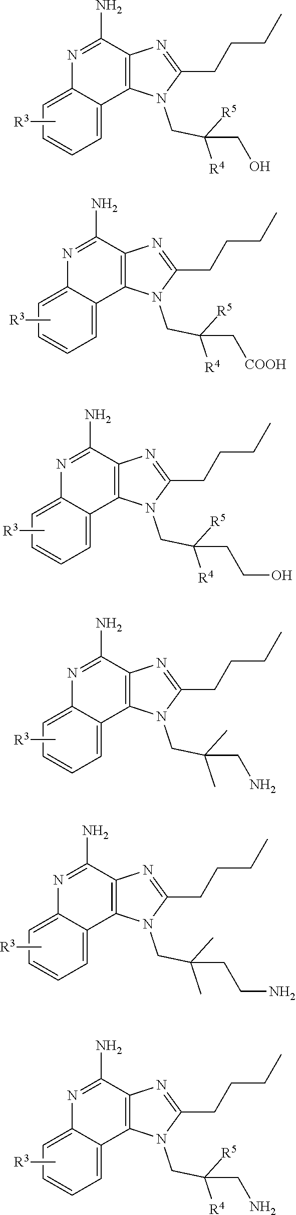

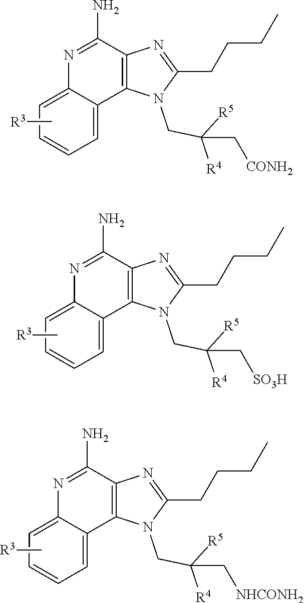

or a pharmaceutically acceptable salt thereof.

In some embodiments, the compound is represented by any one or more of the formulae:

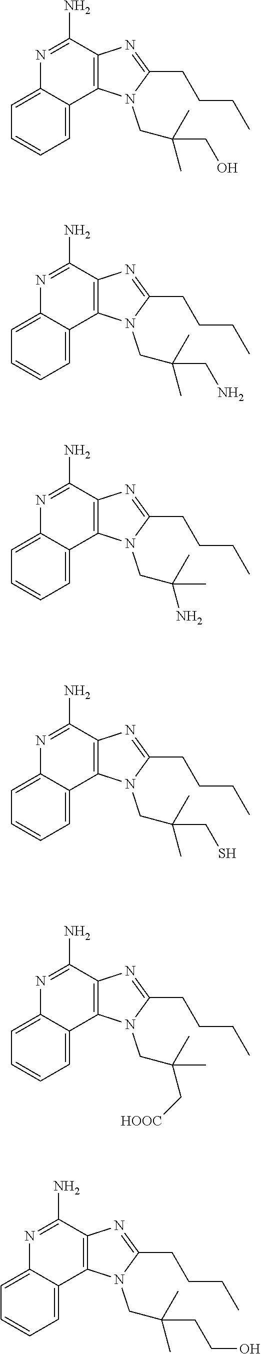 [SMILES: CCCCc1nc2c(N)nc3ccccc3c2n1CC(C)(C)CO ; CCCCc1nc2c(N)nc3ccccc3c2n1CC(C)(C)CN ; CCCCc1nc2c(N)nc3ccccc3c2n1CC(C)(C)N ; CCCCc1nc2c(N)nc3ccccc3c2n1CC(C)(C)CS ; CCCCc1nc2c(N)nc3ccccc3c2n1CC(C)(C)CC(=O)O ; CCCCc1nc2c(N)nc3ccccc3c2n1CC(C)(C)CCO]

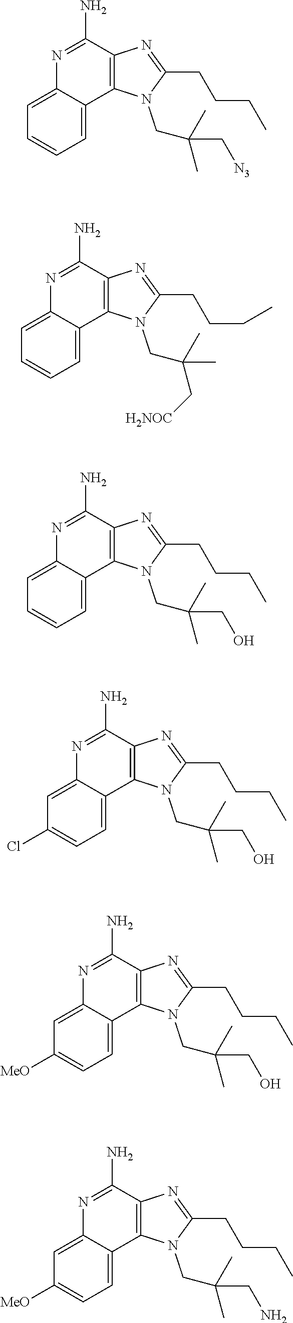 [SMILES: CCCCc1nc2c(N)nc3ccccc3c2n1CC(C)(C)CN=[N+]=[N-] ; CCCCc1nc2c(N)nc3ccccc3c2n1CC(C)(C)CC(N)=O ; CCCCc1nc2c(N)nc3ccccc3c2n1CC(C)(C)CO ; CCCCc1nc2c(N)nc3cc(Cl)ccc3c2n1CC(C)(C)CO ; CCCCc1nc2c(N)nc3cc(OC)ccc3c2n1CC(C)(C)CO ; CCCCc1nc2c(N)nc3cc(OC)ccc3c2n1CC(C)(C)CN]

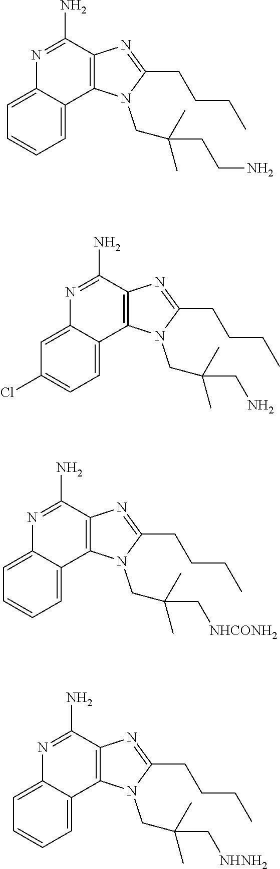

or a pharmaceutically acceptable salt thereof

### Conjugates

The present disclosure further relates to compounds (and radicals thereof) provided herein (e.g., TLR7/8 agonists) that are conjugated, directly or via a linker, to a targeting moiety that targets a pattern recognition receptor of a cell. In some embodiments, the targeting ligand comprises a folate ligand or functional fragment or analog thereof, e.g., pteroyl amino acids. In some embodiments, the linkers are non-releasable. In some embodiments, the conjugates provide targeting molecules having non-releasable linkers thereby reducing systemic exposure of TLR7/8 agonists. In some embodiments, the conjugates provide targeting molecules having non-releasable linkers thereby reducing systemic adverse effects of TLR7/8 agonists.

One embodiment provides a compound represented by the structure of Formula (II):

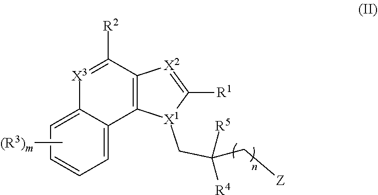

or a pharmaceutically acceptable salt thereof wherein:

R¹, R³, R⁴, R⁵ are each independently a H, an alkyl, an alkoxyl, an alkenyl, an alkynyl, an alicyclic, an aryl, a biaryl, a halo, a heteroaryl, —COR^{2x},

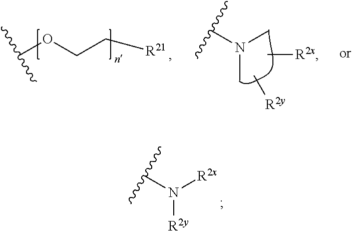

R² is a H, —OH, —NH₂, —NHR^{2x}, N₃, —NH—CH₂—NH₂, —CONH₂, —SO₂NH₂, —NH—CS—NH₂,

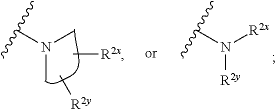

Z is a group of the formula G-L-, G-O—, G-L-O—, G-L-O-alkyl-, G-L-S—, G-SO₂—NH—, G-L-NR^{a}R^{b}—, G-L-S(O)ₓ-alkyl-, G-L-CO—, G-L-aryl-, G-L-NH—CO—NH—, G-L-NH—O—, G-L-NH—NH—, G-L-NH—CS—NH, G-L-C(O)-alkyl-, G-L-SO₂—,

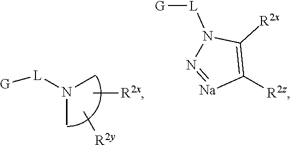

wherein:
L is a linker and G is a folate receptor binding ligand; and
R^{a} and R^{b} are each, independently, H, halo, hydroxy, alkoxy, aryl, amino, acyl or C(O)R^{c}, wherein R is alkyl, aryl, oxy or alkoxy;
x is 0-3 (e.g., an integer that varies from 0-3);
each of R^{2x} and R^{2y} are independently selected from a group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl,
each R^{2z} is independently selected from a group consisting of —NH₂, —NR^{2q}R^{2q′}, R^{2q}, —SO—R^{2q}, and —COR^{2q}, where each R^{2q} and R^{2q′} is independently alkyl or H, and

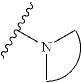 [SMILES: *N(C)C]

is a 3-10 membered N-containing non-aromatic, mono- or bicyclic heterocycle;

R²¹ is H or alkyl; and

n′ is 0-30;

wherein, in Formula II:

X¹, X², X³ are each independently CR^{q} or N, where each R^{q} is independently hydrogen, halogen, or optionally substituted alkyl;

n is 0-30 (e.g., an integer that varies from 0-30); and

m is 0-4.

In certain embodiments of the compound of Formula II, when n is 0, Z is not bound to Formula (II) by an oxygen atom.

One embodiment provides a compound represented by the structure of Formula (IIA):

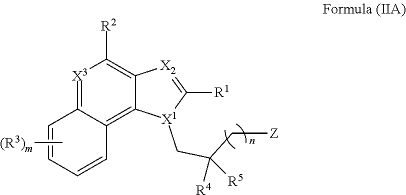

or a pharmaceutically acceptable salt thereof, wherein:

R¹ is optionally substituted alkyl (e.g., acyclic or cyclic) (e.g., optionally substituted with one or more substituent, each substituent independently being halogen, alkyl, heteroalkyl, alkoxy, or cycloalkyl);

R² is H, —OR^{z}, —SO₂N(R^{z})₂, —NR^{2x}R^{2y}, or N₃, where:
R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituent, each substituent independently being oxo, halogen, alkyl, heteroalkyl, alkoxy, or cycloalkyl), and each R^{z} is independently hydrogen, halogen, or optionally substituted alkyl; or
R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);

each R³ is independently halogen, —N₃, —CN, —NO₂, alkyl, heteroalkyl, cycloalkyl, heterocycloalkyl, alkoxy, aryl, heteroaryl, heterocycloalkyl, amino, hydroxy, carboxyl, or thiol, wherein the alkyl, alkoxy, heteroalkyl, cycloalkyl, or heterocycloalkyl is optionally substituted;

R⁴ and R⁵ are each independently alkyl, alkoxy, halogen, or cycloalkyl, wherein the alkyl, alkoxy, and cycloalkyl are optionally substituted;

each X¹, X², and X³ are independently CR^{q} or N, where each R^{q} is independently hydrogen, halogen, or optionally substituted alkyl;

Z is L-G, wherein L is a linker and G is a folate receptor binding ligand;

n is 1-6; and

m is 0-4.

One embodiment provides a compound represented by the structure of Formula (IIA):

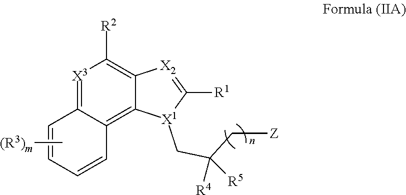

wherein:

R¹ is a C₁-C₆ alkyl optionally substituted with 1-3 substituents, each substituent independently being halogen or C₁-C₆ alkoxy;

R² is —NR^{2x}R^{2y}, where R^{2x} and R^{2y} are each independently a hydrogen or a C₁-C₆ alkyl;

each R³ is independently a halogen, —CN, C₁-C₆ alkyl, C₁-C₆ heteroalkyl, C₃-C₇ cycloalkyl, C₁-C₆ alkoxy, amino, hydroxy, carboxyl, or thiol;

R⁴ and R⁵ are each independently C₁-C₆ alkyl;

each X¹, X², and X³ is N;

Z is G-L- or G-L-O—, wherein L is a linker and G is a folate receptor binding ligand;

n is 1; and

m is 0-4;

or a pharmaceutically acceptable salt thereof.

One embodiment provides a compound of Formula (II), or a pharmaceutically acceptable salt thereof, having the structure of Formula (IIB):

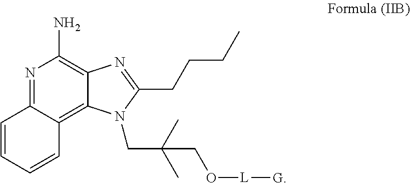

One embodiment provides a compound, or a pharmaceutically acceptable salt thereof, having the structure of Formula (II) or (IIA) or (IIB) wherein n is 1-30. In one embodiment, n is 1-6. In another embodiment, n is 1-3. In another embodiment, n is 1 or 2. In another embodiment, n is 0. In another embodiment, n is 1.

One embodiment provides a compound, or a pharmaceutically acceptable salt thereof, having the structure of Formula (II) or (IIA) or (IIB) wherein R¹ is an optionally substituted alkyl. In one embodiment, R¹ is an optionally substituted C₃-C₆ alkyl. In another embodiment, R¹ is an optionally substituted acyclic C₃-C₆ alkyl. In another embodiment, R¹ is butyl.

One embodiment provides a compound, or a pharmaceutically acceptable salt thereof, having the structure of Formula (II) or (IIA) wherein R² is —NR^{2x}R^{2y}. In one embodiment, R² is NH₂.

One embodiment provides a compound, or a pharmaceutically acceptable salt thereof, having the structure of Formula (II) or (IIA) wherein R³ is H.

One embodiment provides a compound, or a pharmaceutically acceptable salt thereof, having the structure of Formula (II) or (IIA) wherein R⁴ is alkyl. In one embodiment, R⁴ is methyl.

One embodiment provides a compound, or a pharmaceutically acceptable salt thereof, having the structure of Formula (II) or (IIA) wherein R⁵ is alkyl. In one embodiment, R⁵ is methyl.

One embodiment provides a compound, or a pharmaceutically acceptable salt thereof, having the structure of Formula (II) or (IIA) wherein R⁴ and R⁵ are each alkyl. In one embodiment, R⁴ and R⁵ are each methyl.

One embodiment provides a compound, or a pharmaceutically acceptable salt thereof, having the structure of Formula (II) or (IIA) wherein m is 0. In another embodiment, m is 1. In another embodiment, m is 2. In another embodiment, m is 3. In another embodiment, m is 4.

One embodiment provides a compound, or a pharmaceutically acceptable salt thereof, having the structure of Formula (II) or (IIA) wherein X¹, X², and X³ are each N. In one embodiment, X¹ is N. In another embodiment, X² is N. In another embodiment, X³ is N.

One embodiment provides a compound, or a pharmaceutically acceptable salt thereof, having the structure of Formula (II) or (IIA) wherein the compound is represented by the structure:

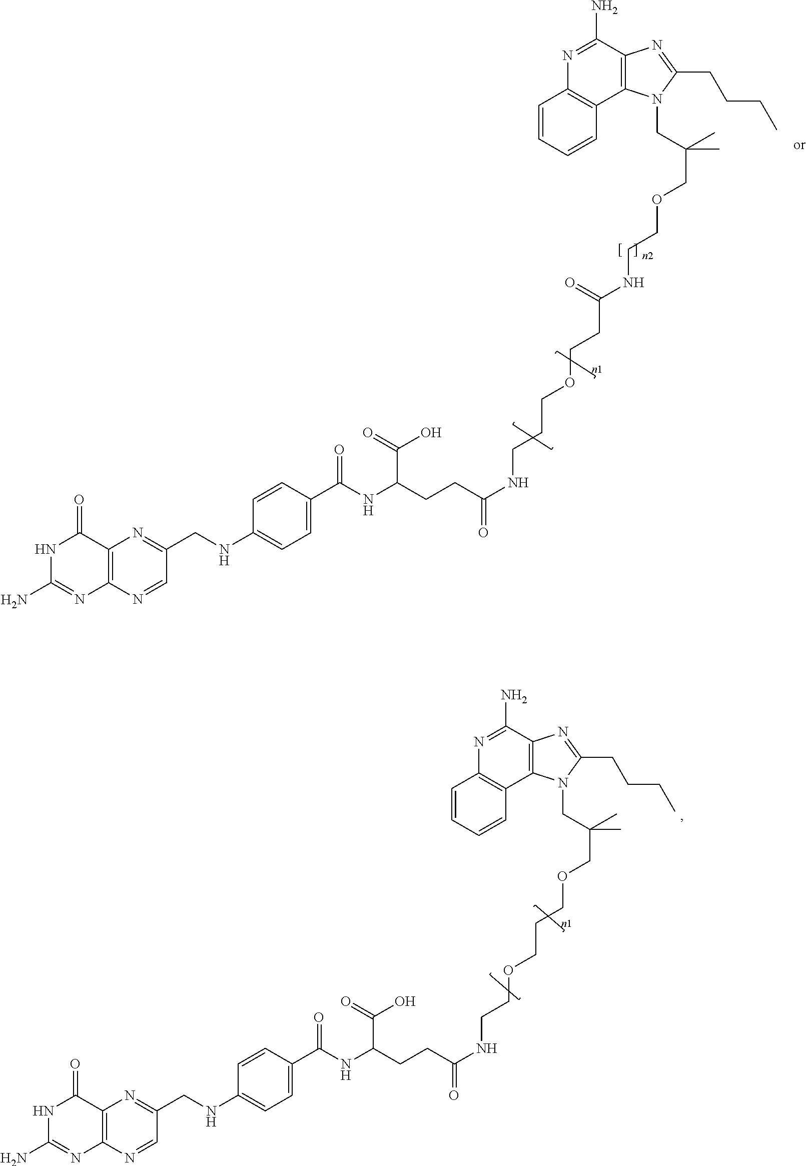

wherein n1 is 0-10 and n2 is 0-10.

One embodiment provides a compound represented by the structure of Formula (III):

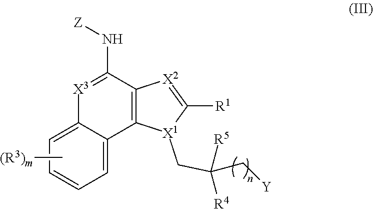

or a pharmaceutically acceptable salt thereof wherein:

R¹, R³, R⁴, R⁵ are each independently a H, an alkyl, an alkoxyl, an alkenyl, an alkynyl, an alicyclic, an aryl, a biaryl, a halo, a heteroaryl, —COR^{2x},

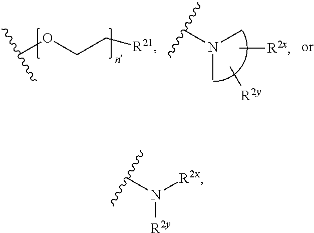

where each of R^{2x}, and R^{2y} are independently selected from a group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and each of R^{2z} is independently selected from a group consisting of —NH₂, —NR^{2q}R^{2q′}—O—R^{2q}, —SO—R^{2q}, and —COR^{2q}, wherein each R^{2q} and R^{2q′} is independently alkyl or H,

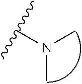 [SMILES: *N(C)C]

is a 3-10 membered N-containing non-aromatic, mono- or bicyclic heterocycle, R²¹ is H or alkyl, and n′ is 0-30;

Z is a group of the formula G-L-, G-L-CO—, G-L-C(O)-alkyl-, wherein L is a linker and G is a folate receptor binding ligand;

X¹, X², X³ are each independently CR^{q} or N, where each R^{q} is independently hydrogen, halogen, or optionally substituted alkyl;

n is 0-30 (e.g., an integer from 0-30); and

n is 0-4.

One embodiment provides a compound represented by the structure of Formula (IIIA):

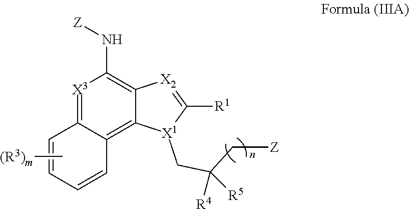

or a pharmaceutically acceptable salt thereof, wherein:

R¹ is optionally substituted alkyl (e.g., acyclic or cyclic) (e.g., optionally substituted with one or more substituent, each substituent independently being halogen, alkyl, heteroalkyl, alkoxy, or cycloalkyl);

Y is H, —OR^{z}, —NR^{2x}R^{2y}, —SR^{z}, —SOR^{z}, —SO₃R^{z}, —N₃, —COR^{z}, —COOR^{z}, —CONR^{z}₂, —COSR^{z}, —SO₂N(R^{z})₂, or —CON(R^{z})₂, where:
R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl (e.g., optionally substituted with one or more substituent, each substituent independently being oxo, halogen, alkyl, heteroalkyl, alkoxy, or cycloalkyl), and each R^{z} is independently hydrogen, halogen, or optionally substituted alkyl; or
R^{2x} and R^{2y} are taken together to form an optionally substituted heterocycloalkyl (e.g., wherein the optionally substituted heterocycloalkyl is a mono- or bicyclic heterocycloalkyl and/or wherein the optionally substituted heterocycloalkyl is a 3-10 membered heterocycloalkyl);

each R³ is independently halogen, —N₃, —CN, —NO₂, alkyl, heteroalkyl, cycloalkyl, heterocycloalkyl, alkoxy, aryl, heteroaryl, heterocycloalkyl, amino, hydroxy, carbonyl, or thiol, wherein the alkyl, alkoxy, heteroalkyl, cycloalkyl, or heterocycloalkyl is optionally substituted;

R⁴ and R⁵ are each independently alkyl, alkoxy, halogen, or cycloalkyl, wherein the alkyl, alkoxy, and cycloalkyl are optionally substituted;

each X¹, X², and X³ are independently CR^{q} or N, and each R^{q} is independently hydrogen, halogen, or optionally substituted alkyl;

Z is L-G, wherein L is a linker and G is a folate receptor binding ligand;

n is 1-6; and

m is 0-4.

One embodiment provides a compound, or a pharmaceutically acceptable salt thereof, having the structure of Formula (III) or (IIIA) wherein n is 1-30. In one embodiment, n is 1-6. In another embodiment, n is 1-3. In another embodiment, n is 1 or 2. In another embodiment, n is 0. In another embodiment, n is 1. In another embodiment, n is 1 and Y is OH. In another embodiment, n is 1 and Y is NH₂.

One embodiment provides a compound, or a pharmaceutically acceptable salt thereof, having the structure of Formula (III) or (IIIA) wherein Y is OH.

One embodiment provides a compound, or a pharmaceutically acceptable salt thereof, having the structure of Formula (III) or (IIIA) wherein Y is NH₂.

One embodiment provides a compound, or a pharmaceutically acceptable salt thereof, having the structure of Formula (III) or (IIIA) wherein n is 1 and Y is OH.

One embodiment provides a compound, or a pharmaceutically acceptable salt thereof, having the structure of Formula (III) or (IIIA) wherein n is 1 and Y is NH₂.

One embodiment provides a compound, or a pharmaceutically acceptable salt thereof, having the structure of Formula (III) or (IIIA) wherein n is 0 and Y is NH₂.

One embodiment provides a compound, or a pharmaceutically acceptable salt thereof, having the structure of Formula (III) or (IIIA) wherein R¹ is an optionally substituted alkyl. In one embodiment, R¹ is an optionally substituted C₃-C₆ alkyl. In another embodiment, R¹ is an optionally substituted acyclic C₃-C₆ alkyl. In another embodiment, R¹ is butyl.

One embodiment provides a compound, or a pharmaceutically acceptable salt thereof, having the structure of Formula (III) or (IIIA) wherein R³ is H.

One embodiment provides a compound, or a pharmaceutically acceptable salt thereof, having the structure of Formula (III) or (IIIA) wherein R⁴ is alkyl. In one embodiment, R⁴ is methyl.

One embodiment provides a compound, or a pharmaceutically acceptable salt thereof, having the structure of Formula (III) or (IIIA) wherein R⁵ is alkyl. In one embodiment, R⁵ is methyl.

One embodiment provides a compound, or a pharmaceutically acceptable salt thereof, having the structure of Formula (III) or (IIIA) wherein R⁴ and R⁵ are each alkyl. In one embodiment, R⁴ and R⁵ are each methyl.

One embodiment provides a compound, or a pharmaceutically acceptable salt thereof, having the structure of Formula (III) or (IIIA) wherein m is 0. In another embodiment, m is 1. In another embodiment, m is 2. In another embodiment, m is 3. In another embodiment, m is 4.

One embodiment provides a compound, or a pharmaceutically acceptable salt thereof, having the structure of Formula (III) or (IIIA) wherein X¹, X², and X³ are each N. In one embodiment, X¹ is N. In another embodiment, X² is N. In another embodiment, X³ is N.

In some embodiments, the compound is represented by any one or more of the structures:

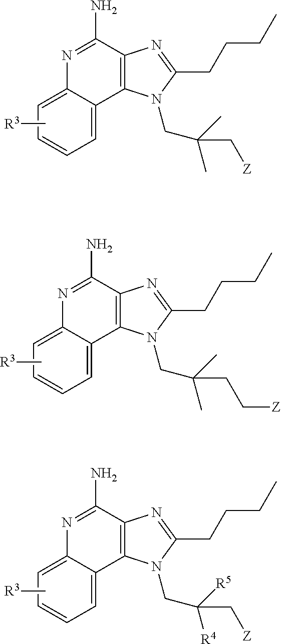

or a pharmaceutically acceptable salt thereof.

In some embodiments, the compound is represented by any one or more of the structures:

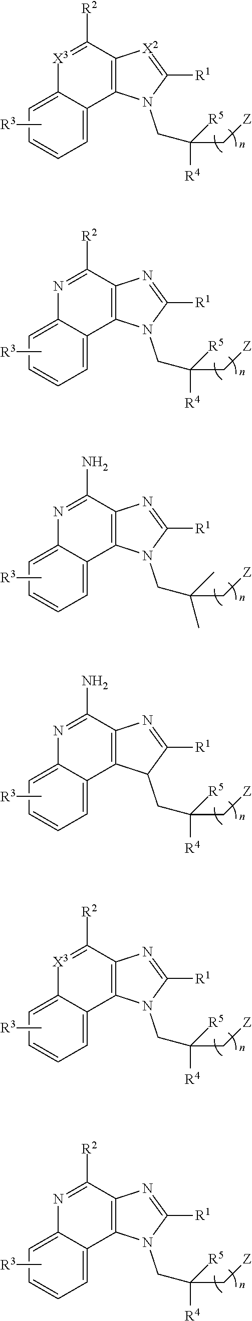

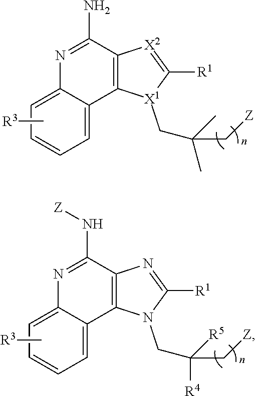

or a pharmaceutically acceptable salt thereof.

In some embodiments, the compound is represented the following structure:

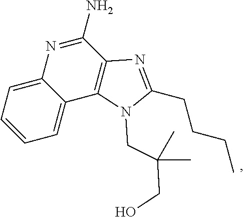 [SMILES: CCCCc1nc2c(N)nc3ccccc3c2n1CC(C)(C)CO]

or a pharmaceutically acceptable salt thereof.

### Linkers

The compounds can comprise an immune modulator or pharmaceutically acceptable salt thereof (e.g., a drug) conjugated with a targeting moiety or a radical thereof through a linker (e.g., optionally comprising a spacer). The linker of the can be releasable or non-releasable. In some instances, the target for a compound comprising a non-releasable linker is the endosome (e.g., of the cell of interest), for example, whereas the target for a releasable linker, in some instances, is the endosome, the cytoplasm, or both (e.g., of the cell of interest).

The term “releasable” in the context of a linker means a linker that includes at least one bond that can be broken (e.g., chemically or enzymatically hydrolyzed) under physiological conditions, such as, for example, by reducing agent-labile, pH-labile, acid-labile, base-labile, oxidatively labile, metabolically labile, biochemically labile, enzymatically labile, or via a p-aminobenzylic-based, multivalent releasable bond. It is appreciated that the physiological conditions resulting in bond breaking do not necessarily include a biological or metabolic process and instead may include a standard chemical reaction, such as a hydrolysis reaction, for example, at physiological pH or as a result of compartmentalization into a cellular organelle, such as an endosome, having a lower pH than cytosolic pH. A cleavable bond can connect two adjacent atoms within the releasable linker and/or connect other linker portions or the targeting moiety and/or the drug, as described herein, for example, at either or both ends of the releasable linker. In some instances, the releasable linker is broken into two or more fragments. In some instances, the releasable linker is separated from the targeting moiety. In some embodiments, the targeting moiety and the immune modulator are released from each other, and the immune modulator becomes active.

In contrast, the term “non-releasable” in the context of a linker means a linker that includes at least one bond that is not easily or quickly broken under physiological conditions. In some embodiments, a non-releasable linker comprises a backbone that is stable under physiological conditions (e.g., the backbone is not susceptible to hydrolysis (e.g., aqueous hydrolysis or enzymatic hydrolysis)). In some embodiments, a compound comprising a non-releasable linker does not release any component of the compound (e.g., a targeting ligand (e.g., an FA-ligand) or an immune modulator (e.g., a TLR7 agonist)). In some embodiments, the non-releasable linker lacks a disulfide bond (e.g., S—S) or an ester in the backbone. In some embodiments, the compound comprises a targeting moiety and an immune modulator connected by a backbone that is substantially stable for the entire duration of the compound's circulation (e.g., during endocytosis into the target cell endosome). In some embodiments, the compound comprising the non-releasable linker is particularly beneficial when the immune modulator targets TLRs, nucleotide-binding oligomerization domain-(NOD)-like receptors, and/or other pattern recognition receptors present within the endosome of a cell. The non-releasable linker can comprise an amide, ester, ether, amine, and/or thioether (e.g., thio-maleimide). While specific examples are provided herein, it will be understood that any molecule(s) may be used in the non-releasable linker provided that at least one bond that is not easily or quickly broken under physiological conditions is formed.

In some embodiments, a non-releasable linker comprises a linker that, at a neutral pH, for example, less than ten percent (10%) (e.g., less than 5%, less than 4%, less than 3%, less than 2%, less than 1%, less than 0.1%, less than 0.01%, or less than 0.001%) will hydrolyze in an aqueous (e.g., buffered (e.g., phosphate buffer) solution) within a period of time (e.g., 24 hours). In some embodiments, where a non-releasable linker is employed, less than about ten percent (10%), and preferably less than five percent (5%) or none, of the conjugate administered releases the free drug (e.g., in systemic circulation prior to uptake by the targeted cells/tissue). In some embodiments, within one (1) hour of administration, less than five percent (5%) of the free drug is released from the conjugate while the compound is in systemic circulation.

In some embodiments, the targeting moiety does not cleave from the drug/immune modulator for the compound to be therapeutically effective in vivo. This can be advantageous as it can allow for the use of targeting compounds and compositions comprising potent drugs (e.g., TLR7 and TLR7/8 agonists), for example, because only a negligible amount (if any) of the drug (e.g., immune modulator, e.g., TLR7 or TLR7/8 agonist) is released (e.g., systemically) prior to the targeted delivery of the compound. In some embodiments, tuning the releasing properties of active components is a difficult aspect of the preparation of effective pharmaceutical compositions. In some embodiments, the compounds comprising the non-releasable linkers provided herein avoid the difficulties of the preparation of effective pharmaceutical compositions (e.g., by removing the necessity of timing the release). In some embodiments, the immune modulator/warhead of the compound provided herein is active when bound (e.g., conjugated to the targeting conjugate). In some embodiments, while the warhead/immune modulator is active, the non-releasable linker and the targeting moiety prevent the release of toxic cytokines (e.g., by the subject's body) that activate the immune system (such as, for example, IL-6) (e.g., because the compound is specifically targeted (using, for example, folate or an analog thereof)). In certain instances, the immune modulator cannot access the appropriate (e.g., targeted) receptor within the endosome of the cell until the compound binds to the targeted receptor (for example, a folate receptor), for example, even though the warhead/immune modulator of the compound is active when connected to the non-releasable linker.

Both releasable and non-releasable linkers can be engineered to optimize biodistribution, bioavailability, and PK/PD (e.g., of the compound) and/or to increase uptake (e.g., of the compound) into the targeted tissue pursuant to methodologies commonly known in the art or hereinafter developed such as through PEGylation and the like. In some embodiments, the linker is configured to avoid significant release of a pharmaceutically active amount of the drug in circulation prior to capture by a cell (e.g., a cell of interest (e.g., a macrophage in fibrotic or cancer tissue to be treated)).

In some embodiments, the compounds comprising releasable linkers of the present disclosure can be designed to diffuse across the membrane of the endosome and, for example, into the cytoplasm of the targeted cell. In some embodiments, releasable linkers can be designed such that the immune modulator is not released until the compound reaches the cytoplasm.

In some embodiments, a conjugate provided herein comprises a releasable linker (e.g., to facilitate the release of the immune modulator in the cytoplasm). The releasable linker can, for example, prevent the release of the immune modulator until after the targeting moiety binds the appropriate target (e.g., a macrophage folate receptor), is internalized into the endosome of the targeted cell, and/or diffuses into the cytoplasm (e.g., which is where the desired pattern recognition receptor is located). In some embodiments, the releasable linker releases the immune modulator within the endosome.

In some embodiments, linkers can comprise one or more spacers (e.g., to facilitate a particular release time, facilitate an increase in uptake into a targeted tissue, and/or optimize biodistribution, bioavailability, and/or PK/PD of a compound). A spacer can comprise one or more of alkyl chains, polyethylene glycols (PEGs), peptides, sugars, peptidoglycans, clickable linkers (e.g., triazoles), rigid linkers such as poly-prolines and poly-piperidines, and the like.

In some embodiments, a linker comprising PEG₁₂ significantly reduces—if not altogether avoids—nonspecific uptake of the compounds provided herein (e.g., into a non-targeted organ (e.g., into the liver and/or kidneys of a subject following administration)). In some embodiments, the compounds avoid delivery to the liver and kidneys. In some embodiments, the targeting moieties (in their free form, a radical thereof, or a conjugate thereof) do not bind with uptake receptors on non-targeted cells (e.g., provided the organs are not the targeted sites, and, as such, stimulation of the immune complex in those organs can be avoided, which is highly beneficial in a clinical context).

In some embodiments, a conjugate comprising anon-releasable linker reduces or eliminates toxicity of a component released from the conjugate in its free form (e.g., a free form of a compound and/or ligand provided herein).

One embodiment provides a compound, or a pharmaceutically acceptable salt thereof, having the structure of Formula (II), (IIA), (IIB), (III) or (IIIA) wherein L is a cleavable linker.

In some embodiments, the one or more linkers of the compound provided herein can comprise PEG, a PEG derivative, or any other linker known in the art or hereinafter developed that can achieve the purpose set forth herein. In some embodiments, the linker is repeated n times, where n is a positive integer. For example, and without limitation, n can be any integer selected from a range of 1-16, 1-32, 1-64, or 1-96. The number of repeats in the linker can be selected to achieve the desired functionality, size, and/or potency of the compound and/or in view of the desired application. In some embodiments, the one or more of the linkers comprise one or more spacers (e.g., which may also be used to specifically design characteristics of the compound).

In some embodiments, the linker is a hydrolyzable linker. In some embodiments, the linker is a non-hydrolyzable linker. In some embodiments, the linker is an optionally substituted heteroalkyl. In some embodiments, the linker is a substituted heteroalkyl comprising at least one substituent selected from the group consisting of alkyl, hydroxyl, oxo, PEG, carboxylate, and halo. In some embodiments, the linker comprises a spacer (e.g., as described elsewhere herein).

One embodiment provides a compound, or a pharmaceutically acceptable salt thereof, having the structure of Formula (II), (IIA), (IIB), (III) or (IIIA) wherein L is a hydrolyzable linker (e.g., amide, ester, ether, or sulfonamide).

In another embodiment, L is an optionally substituted heteroalkyl. In some embodiments, the heteroalkyl is unsubstituted. In other embodiments, the heteroaryl is substituted with at least one substituent selected from the group consisting of alkyl, hydroxyl, acyl, PEG, carboxylate, and halo. In another embodiment, L is a substituted heteroalkyl with at least one disulfide bond in the backbone thereof.

In another embodiment, L is a peptide or a peptidoglycan with at least one disulfide bond in the backbone thereof.

In another embodiment, L is a cleavable linker that can be cleaved by enzymatic reaction, reaction oxygen species (ROS) or reductive conditions.

In some embodiments, L has the formula: —NH—CH₂—CR⁶R⁷—S—S—CH₂—CH₂—O—CO—, wherein R⁶ and R⁷ are each, independently, H, alkyl, or heteroalkyl.

In some embodiments, L is a group or comprises a group of the formulae:

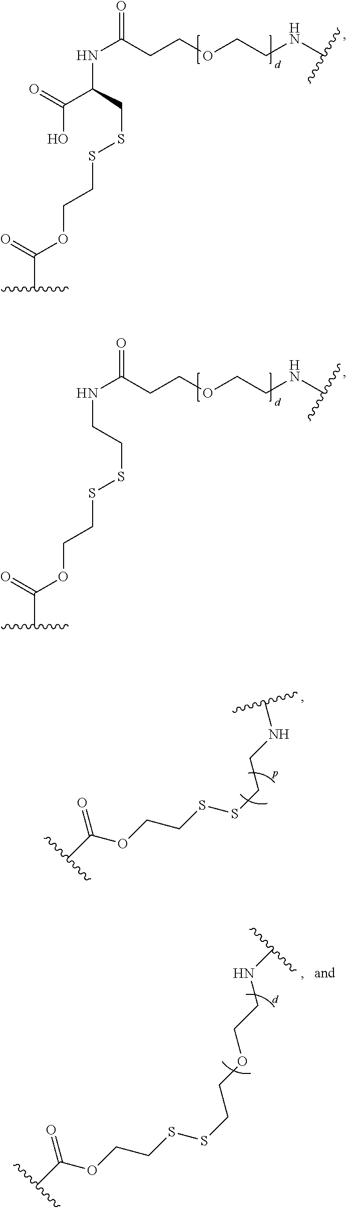

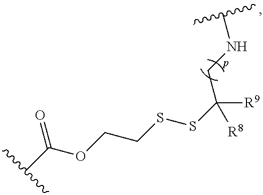

wherein,

p is an integer from 0 to 30;

d is an integer from 1 to 40; and

R⁸ and R⁹ are each, independently, an H, an alkyl, a heterocyclyl, a cycloalkyl, an aryl, or a heteroalkyl.

One embodiment provides a compound, or a pharmaceutically acceptable salt thereof, having the structure of Formula (II), (IIA), (IIB), (III) or (IIIA), wherein L is a non-releasable linker.

One embodiment provides a compound, or a pharmaceutically acceptable salt thereof, having the structure of Formula (II), (IIA), (IIB), (III) or (IIIA), wherein L is a non-hydrolyzable linker.

In some embodiments, L is selected from the group consisting of alkylene, heteroalkylene, —O— alkynylene, alkenylene, acyl, aryl, heteroaryl, amide, oxime, ether, ester, triazole, PEG, and carboxylate.

In one embodiment, L is or comprises an alkyl ether. In another embodiment, L is or comprises an amide. In another embodiment, L is or comprises a peptide or a peptidoglycan. In another embodiment, L is or comprises an amino acid. In another embodiment, L is or comprises a PEG (e.g., —OCH₂—CH₂—O—). In another embodiment, L is or comprises polysaccharide. In another embodiment, L is or comprises a group represented by the structure:

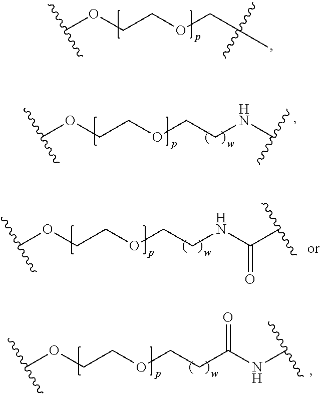

wherein w is 0-5 and p is 1-30.

In one embodiment, L is or comprises a linker moiety selected from the following list:

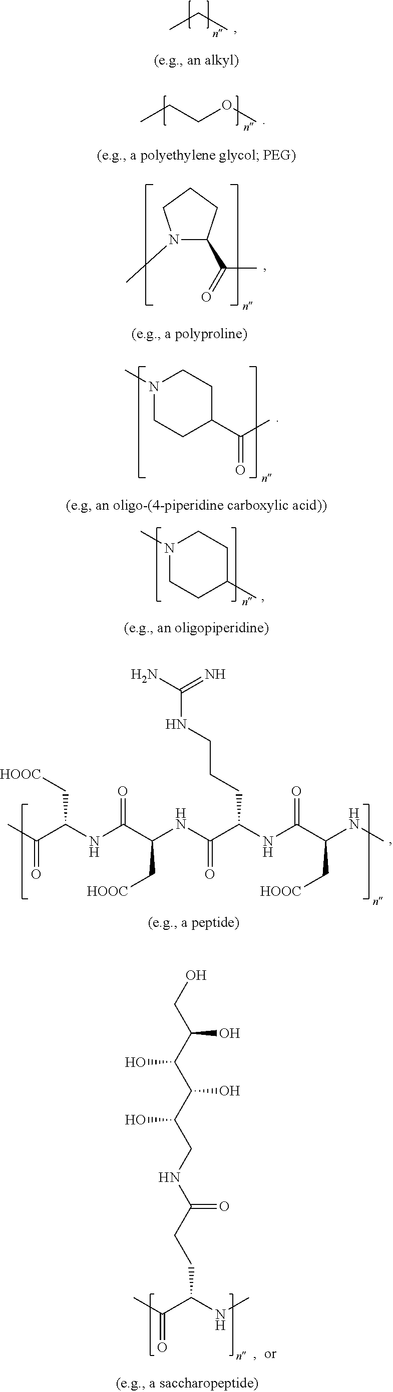

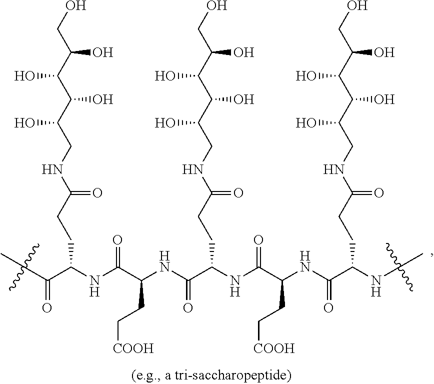

wherein n″ is 0-30 (e.g., n″ is an integer that varies from 0-30).

In some embodiments, the linker comprises —CONH—CH(COOH)—CH₂—S—S—CH₂—R^{a}R^{b}—O—CO—, —CONH—CH(COOH) R^{a}R^{b}—O—CO—, —C(O)NHCH(COOH)(CH₂)₂—CONH—CH(COOH)CR^{a}R^{b}—O—CO— or —C(O)NHCH(COOH)(CH₂)₂—CONH—CH(COOH)—CH₂—S—S—CH₂—R^{a}R^{b}—O—CO—, wherein Rₐ and R_{b} are independently H, alkyl, or heteroalkyl (e.g., PEG).

In some embodiments, the linker L comprises a structure of:

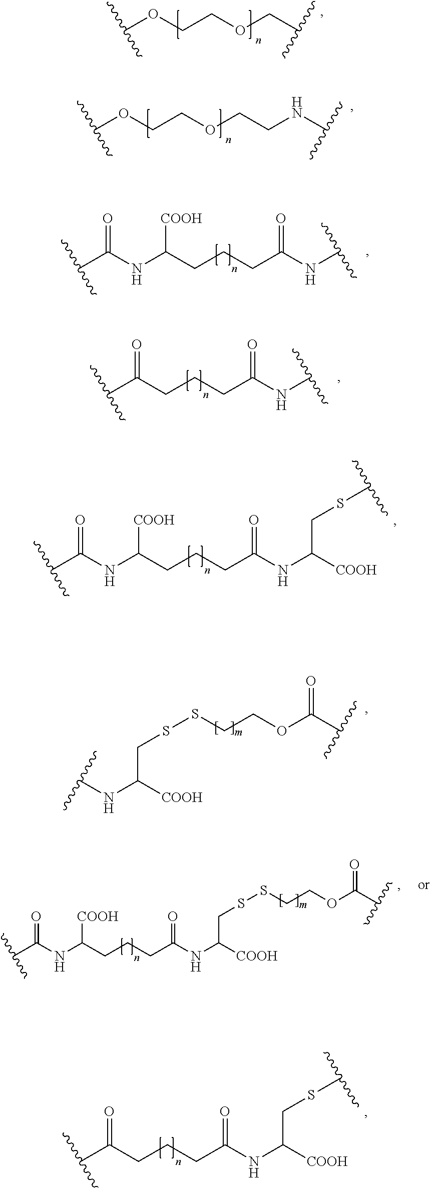

wherein n and m are each independently an integer from 0 to 10.

In some embodiments, the linker L comprises a structure of:

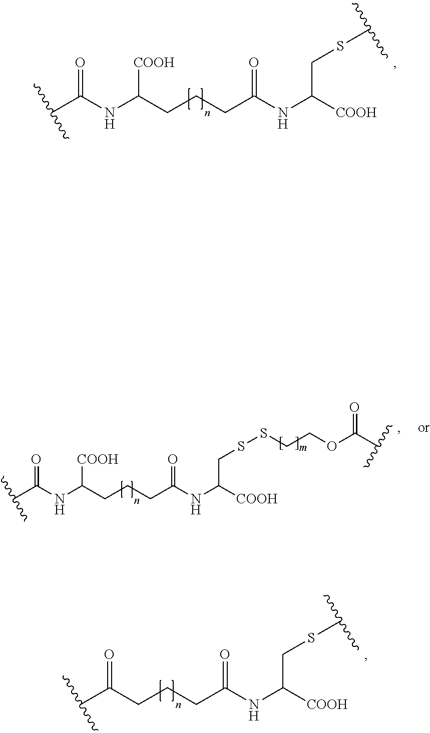

wherein n and m are each independently an integer from 0 to 10.

In some embodiments, the linker L comprises a structure of:

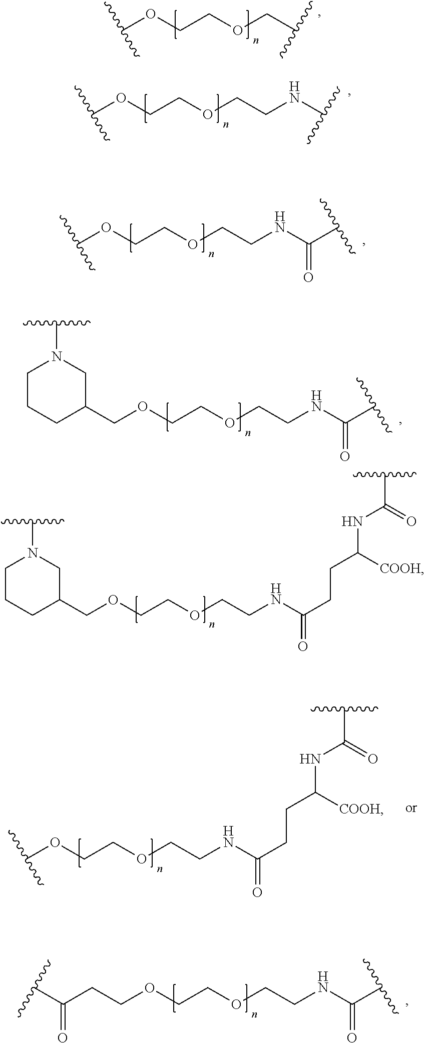

wherein n is an integer from 1 to 32.

In some embodiments, the linker comprises the structure of:

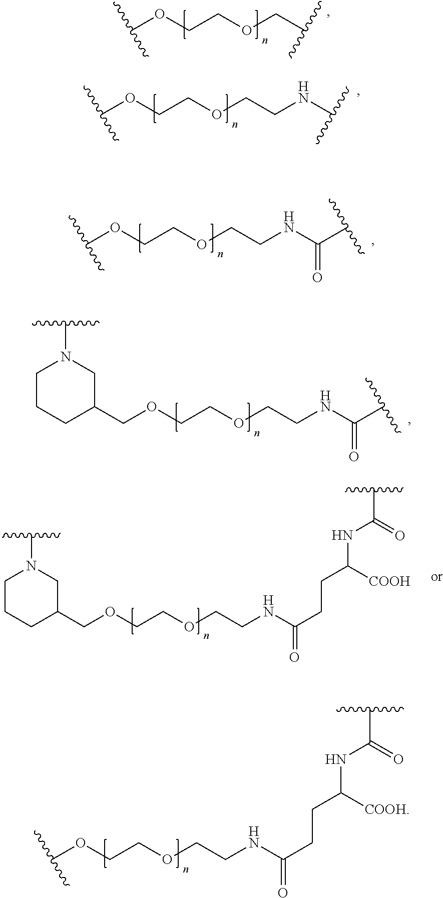

In some embodiments, the linker is a bivalent linker (e.g., connecting two groups). In some embodiments, the linker is a releasable linker. In some embodiments, the linker is a non-releasable linker.

The linker present in the compounds described herein can be any suitable linker. For example, in some embodiments, the linker is a hydrophilic linker, such as a linker that comprises one or more of an amino acid (which are the same or different), an alkyl chain, a PEG monomer, a PEG oligomer, a PEG polymer, or a combination of an any of the foregoing. In some embodiments, the linker comprises an oligomer of peptidoglycans, glycans, or anions.

For a linker that comprises one or more PEG units, all carbon and oxygen atoms of the PEG units are part of the backbone unless otherwise specified. A cleavable bond for a releasable linker is part of the backbone. The “backbone” of the linker L is the shortest chain of contiguous atoms forming a covalently bonded connection between G¹ and G².

In some embodiments, a polyvalent linker has a branched backbone, with each branch serving as a section of backbone linker until reaching a terminus.

### Folate Receptor Binding Ligands

In some instances, toxicities associated with systemic administration of at least the conventional drugs identified herein have precluded their practical application with respect to treating fibrotic diseases and disorders, cancers, and other disease states. For example, TLR agonists may not be tolerated by an individual and, in some instances, can result in the death of a subject (e.g., if administered systemically via conventional modalities). In some embodiments, the compounds such as, for example, those having Formula (I), (IA), (II), (IIA), (IIB), (III), and/or (IIIA), are potent and can be used with a mechanism for circumventing systemic toxicity (e.g., the targeting moieties provided herein and/or releasable and non-releasable linkers).

In some instances, the immune modulator or pharmaceutically acceptable salt thereof is conjugated to a targeting moiety. In some embodiments, the targeting moiety comprises a ligand or other atom or molecule that targets a particular area or tissue of an individual (e.g., with high specificity) and, in certain instances, can, for example, comprise hormones, antibodies, and/or vitamins. As described in further detail below, in at least one embodiment, the targeting moiety comprises a molecule that has (e.g., a high) affinity for folate receptor β (FR-β). In some instances, the targeting moiety has a specific affinity for any receptor that is particular to cells or tissues of a fibrotic disease or disorder, or a cancer, as appropriate.

In some instances, FR-β is significantly upregulated in activated myeloid cells (e.g., predominantly activated monocytes and M2-like macrophages), for example, with recorded data to date supporting that FR-β is only induced in cells of myelogenous origin following exposure to anti-inflammatory or proinflammatory stimuli. The folate receptor can be upregulated in (e.g., more than 90%) of non-mucinous ovarian carcinomas. In certain instances, the folate receptor is present in kidney, brain, lung, and breast carcinoma. Although there are a number of cancers that do not themselves express the folate receptor in sufficient numbers to provide the desired specificity, cancerous tumors do express myeloid-derived suppressor cells (MDSCs), for example, which do express FR-β and can be targeted by a targeting moiety. In some embodiments, folate receptors are not substantially present (e.g., present only at extremely low levels) in healthy (non-myeloid) tissues (e.g., whether lungs, liver, spleen, heart, brain, muscle, intestines, pancreas, bladder, etc.). In some instances, uptake of folate-targeted imaging agents is in, for example, inflamed tissues, malignant lesions, and the kidneys. In certain instances, subjects devoid of cancer only retain folate-targeted drugs in the kidneys and sites of inflammation. In some instances, the discrepancy in folate receptor expression provides a mechanism for selectively targeting fibrotic cancer cells.

In some embodiments, the compounds, compositions, and methods leverage the limited expression of FR-β to target/localize systemically administered potent compounds (e.g., conjugates or drugs) to fibrotic and/or cancerous tissue. In some instances, the compounds are delivered directly to FR-β expressing cells, for example, which advantageously prevents the systemic activation of the immune system and, for example, can avoid (e.g., at least a portion of) the toxicity that has heretofore prevented systemic use of non-targeting compounds (e.g., drugs). In some embodiments, the methods are used to treat fibrotic diseases and/or cancers, for example, regardless of whether the cancer expresses the folate receptor. In some embodiments, folic acid and other folate receptor binding ligands (or radicals thereof), such as, for example folate, are used as targeting moieties, since for example, they have affinity for FR-3.

Folic acid is a member of the B family of vitamins and can play an essential role in cell survival by participating in the biosynthesis of nucleic and amino acids. Folic acid can enhance the specificity of conjugated immune modulator drugs by targeting activated myeloid cells and conjugated anti-cancer drugs by targeting folate receptor-positive cancer cells. Provided herein are compounds comprising a folate ligand (or radical thereof), or a functional fragment or analog thereof, as a targeting moiety and an immune modulator (e.g., TLR7 or TLR7/8 agonist). In some instances, TLR7 and TLR7/8 are present in the endosome. In some embodiments, the compound, or radical thereof, binds to a TLR (e.g., TLR7 or TLR7/8).

A pyrido[2,3-d]pyrimidine analog ligand (e.g., or radical thereof), a functional fragment or analog thereof, or any other molecule, fragment or atom with an affinity (for example, and without limitation, a high specificity) for FR-β may alternatively be used as the targeting moiety (or radical thereof). For example, such folate analog molecules may have a relative affinity for binding FR-β of about 0.01 or greater as compared to folic acid at a temperature about 20° C./25° C./30° C./physiological. Similarly, a Galectin-3 ligand, a translocator protein (TSPO) ligand, and any other ligand or targeting moiety with a highly specific affinity for fibrotic and/or cancerous cells or tissue may be employed.

Specific examples of suitable targeting moieties (or radicals thereof) will now be provided; however, it will be understood that the targeting moiety (or radical thereof) can comprise any ligand (or radical thereof) useful to target FR-β and is not limited to the structures specified herein. The ligand (or radical thereof) can bind to FR-β.

One embodiment provides a compound, or a pharmaceutically acceptable salt thereof, having the structure of Formula (II), (IIA), (IIB), (III) or (IIIA) wherein G is a folate receptor binding ligand. In one embodiment, G is or is derived from folate, folic acid, or a functional fragment or derivative thereof. In one embodiment, G is a folate or folate derivative. In another embodiment, G is a pteroic acid or pteroyl derivative.

In some embodiments, G is a reduced folate. In some embodiments, G is a naturally occurring folate. In some embodiments, G is selected from the group consisting of a 5-methyltetrahydrofolate (5-MTHF), a 5-formyltetrahydrofolate (5-formyl-THF), a 10-formyltetrahydrofolate (10-formyl-THF), a 5,10-methylenetetrahydrofolate (5,10-methylene-THF), a 5,10-methenyltetrahydrofolate (5,10-methenyl-THF), a 5,10-formiminotetrahydrofolate (5,10-formimino-THF), a 5,6,7,8-tetrahydrofolate (THF), and a dihydrofolicacid (DHF).

One embodiment provides a compound, or a pharmaceutically acceptable salt thereof, having the structure of Formula (II), (IIA), (IIB), (III) or (IIIA), wherein G is a group or comprise a group of Formula (IV):

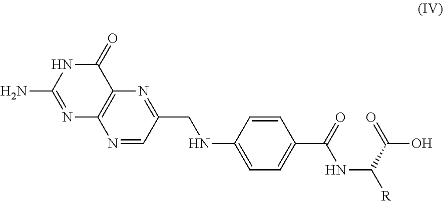

wherein, each R is or comprises, independently,

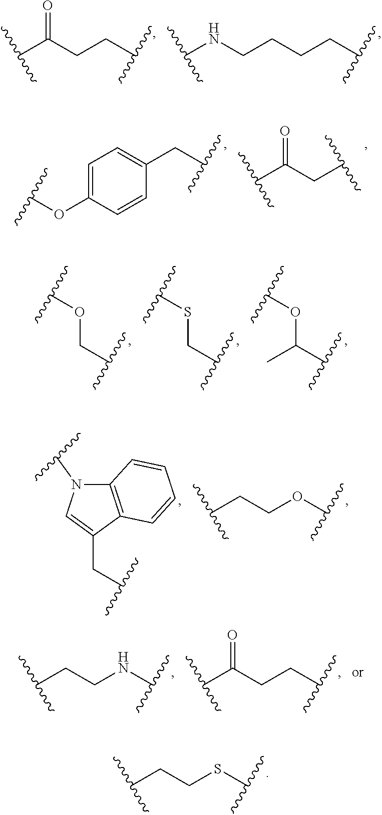

or R is a naturally occurring or unnatural amino acid or its derivative or fragments.

One embodiment provides a compound, or a pharmaceutically acceptable salt thereof, having the structure of Formula (II), (IIA), (IIB), (III) or (IIIA), wherein G is a radical (e.g., a group or comprises a group) having the structure of Formula (V):

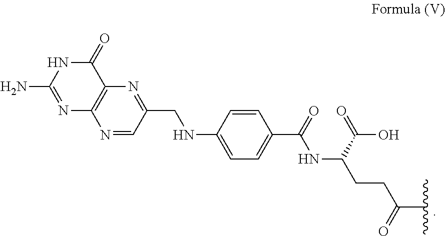

One embodiment provides a compound, or a pharmaceutically acceptable salt thereof, having the structure of Formula (II), (IIA), (IIB), (III) or (IIIA), wherein G is a radical (e.g., a group or comprises a group) having the structure of Formula (VI):

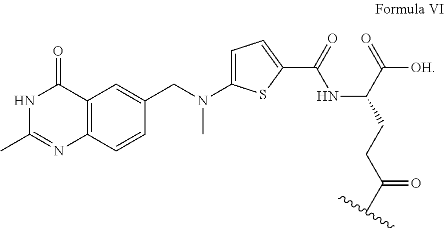 [SMILES: *C(=O)CC[C@H](NC(=O)c1ccc(N(C)Cc2ccc3nc(C)[nH]c(=O)c3c2)s1)C(=O)O]

One embodiment provides a compound, or a pharmaceutically acceptable salt thereof, represented by one of the following structures:

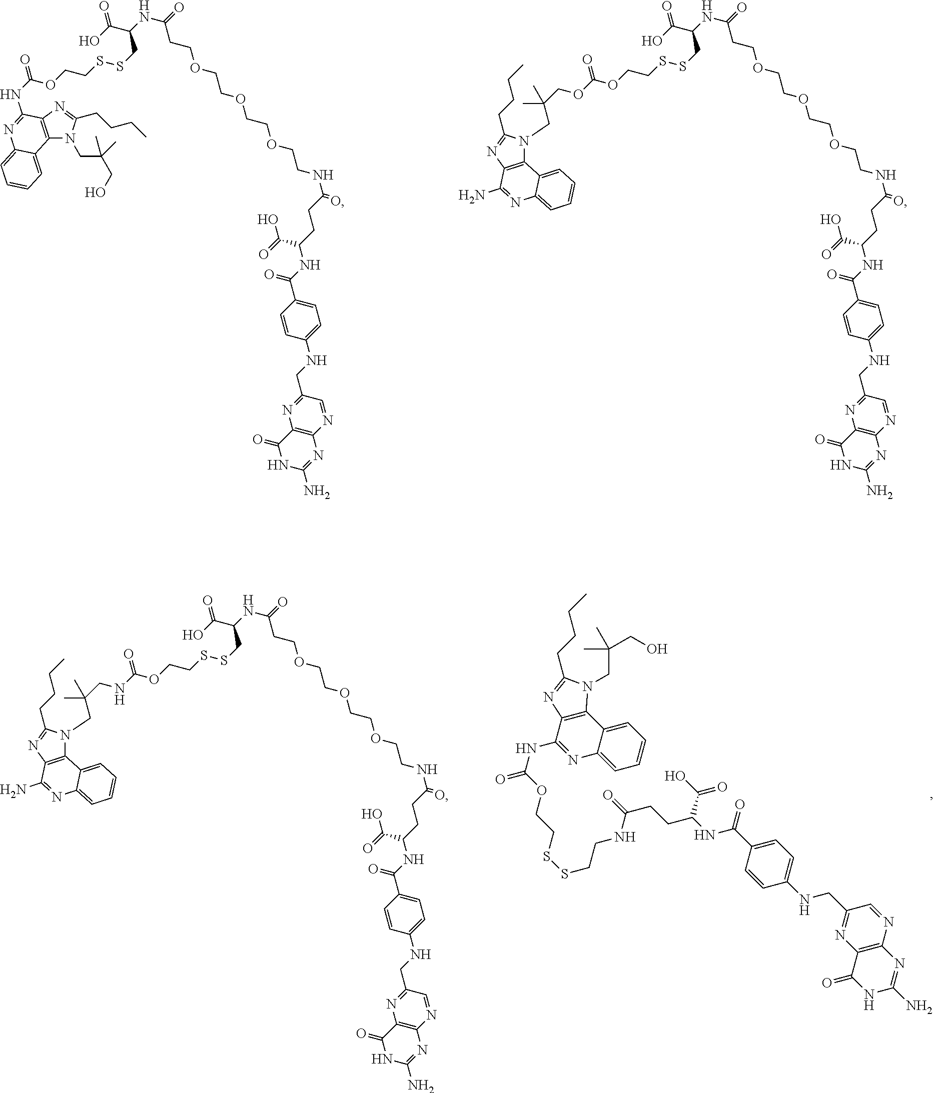

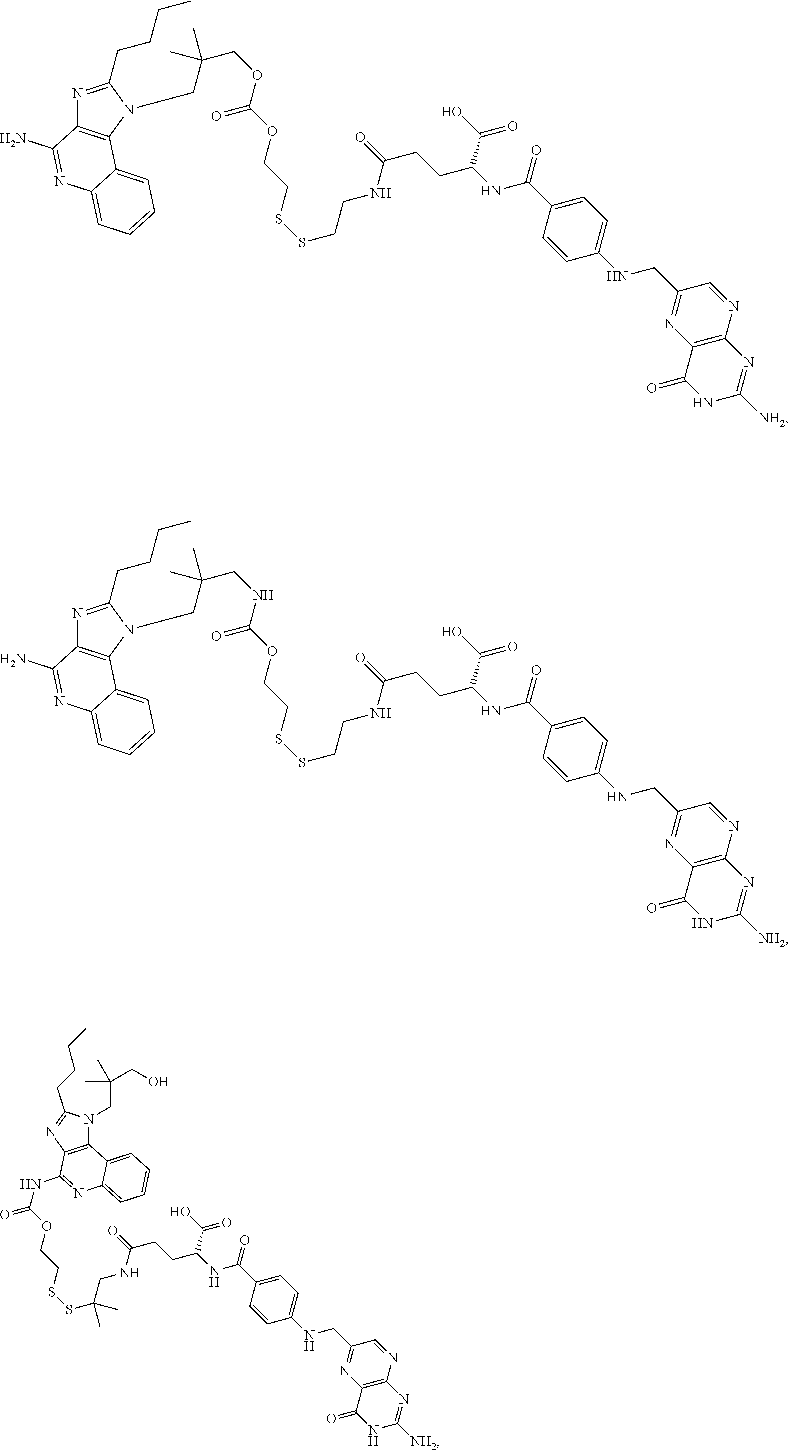

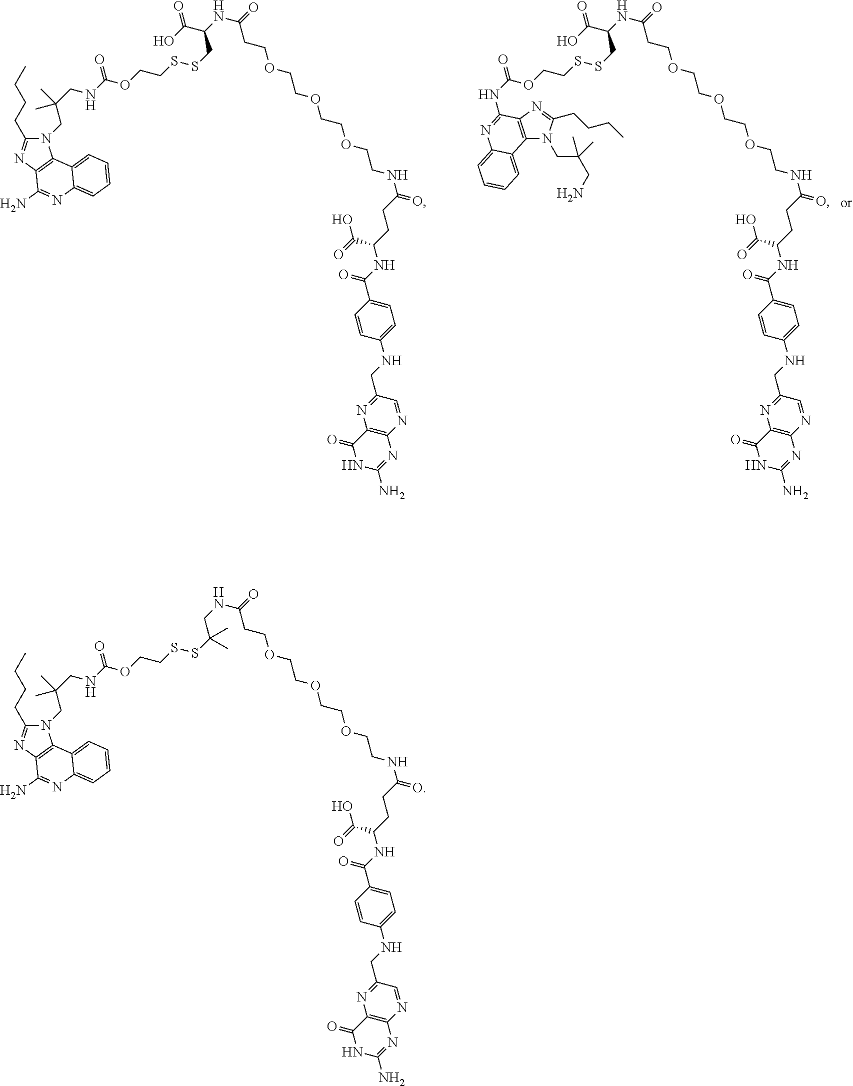

One embodiment provides a compound, or a pharmaceutically acceptable salt thereof, represented by one of the following structures:

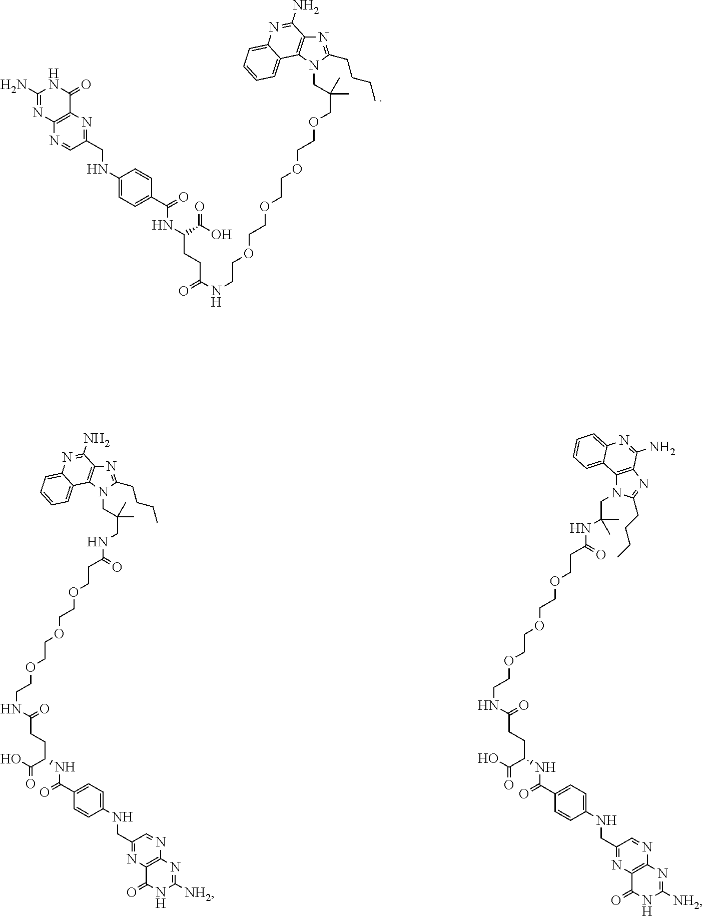

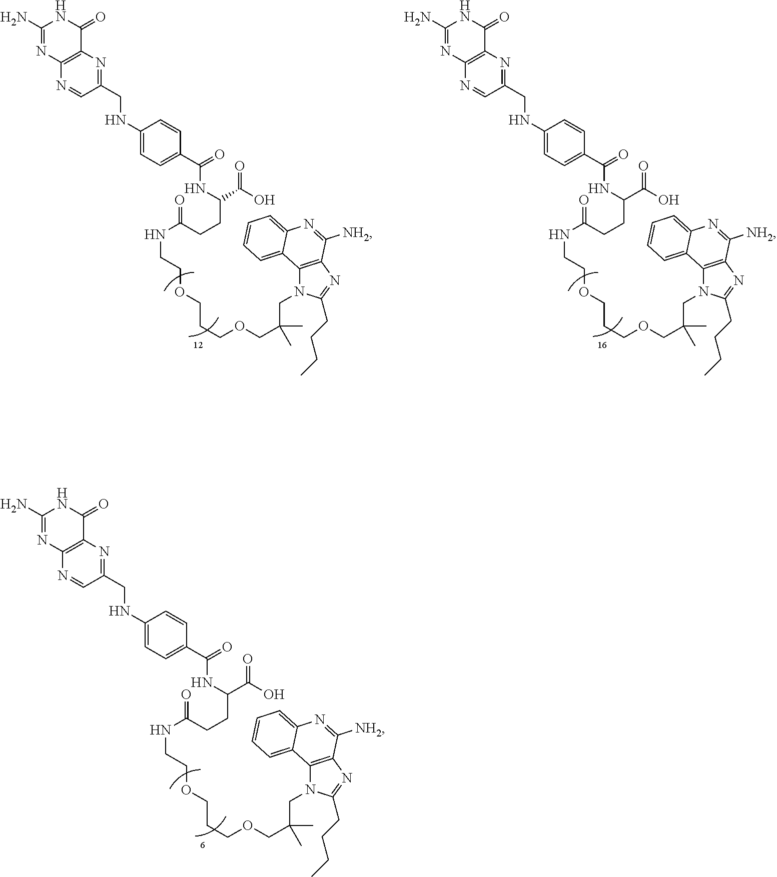

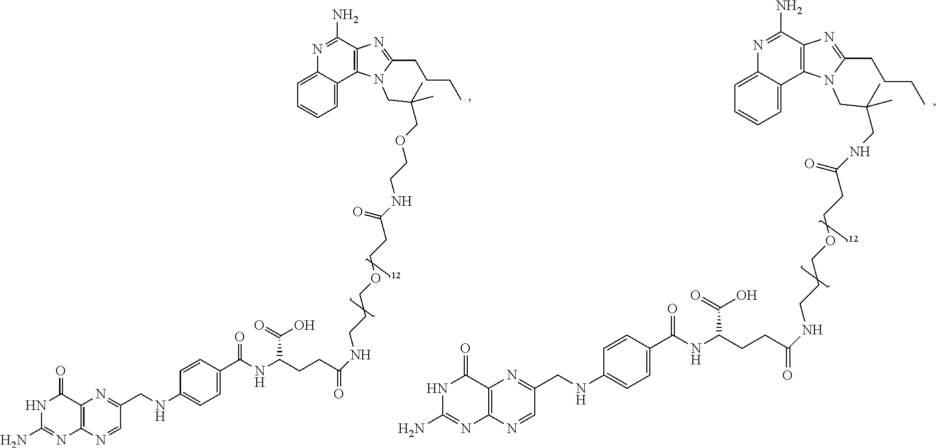

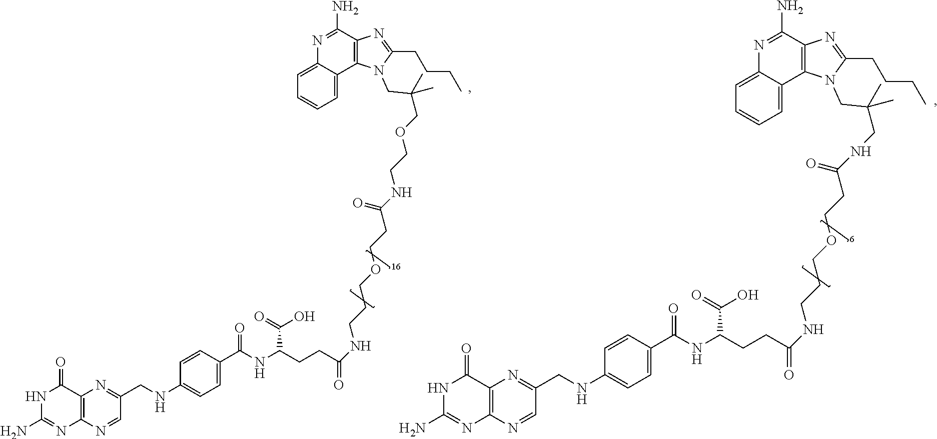

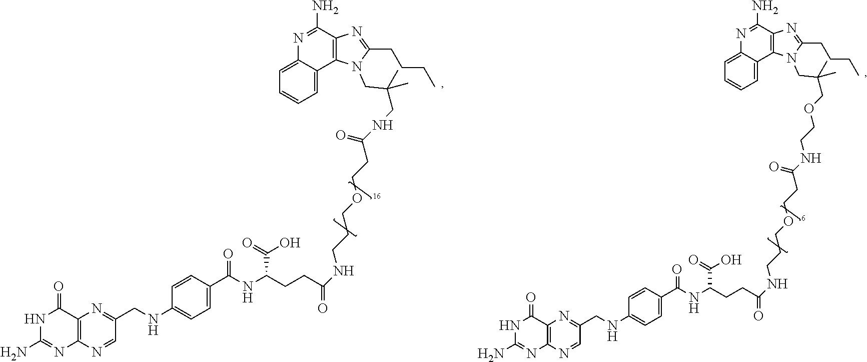

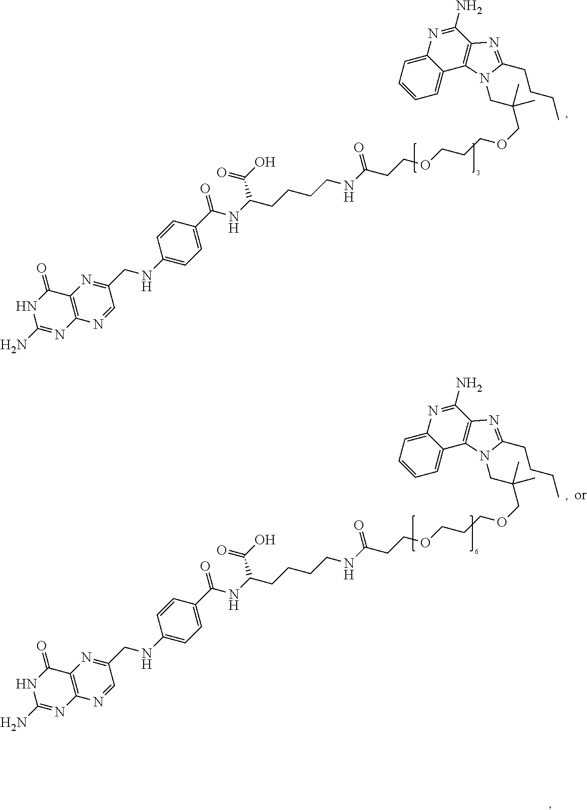

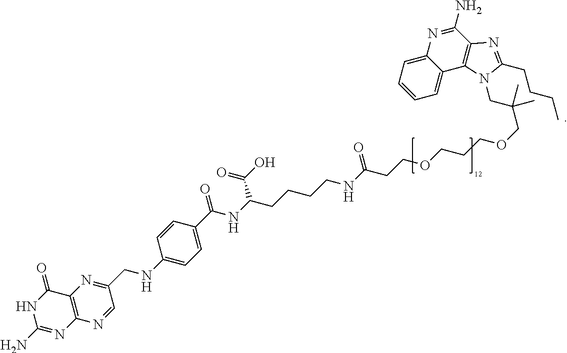

One embodiment provides a compound (Compound 5) (e.g., a folate-PEG₃-TLR7-1A comprising a releasable linker), or a pharmaceutically acceptable salt thereof, having the structure:

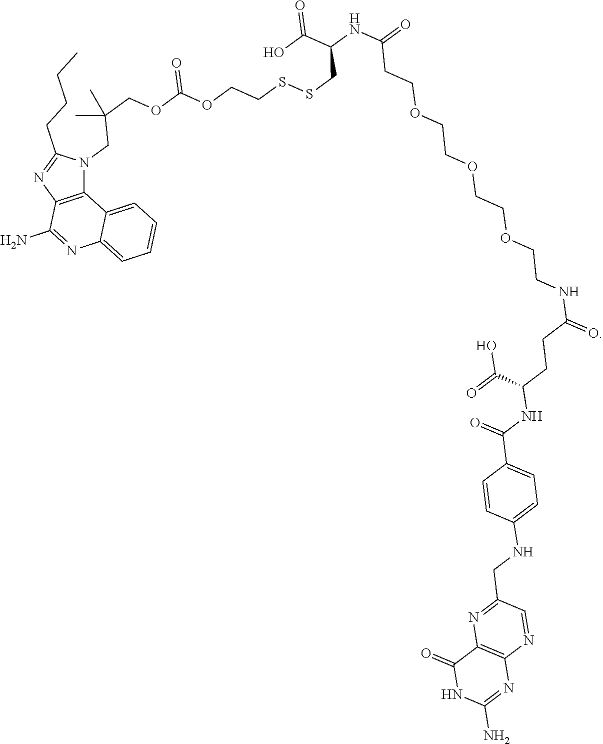 [SMILES: CCCCc1nc2c(N)nc3ccccc3c2n1CC(C)(C)COC(=O)OCCSSC[C@H](NC(=O)CCOCCOCCOCCNC(=O)CC[C@H](NC(=O)c1ccc(NCc2cnc3nc(N)[nH]c(=O)c3n2)cc1)C(=O)O)C(=O)O]

One embodiment provides a compound (Compound 4) (e.g., a folate-PEG₃-TLR7-1A comprising a non-releasable linker), or a pharmaceutically acceptable salt thereof, having the structure:

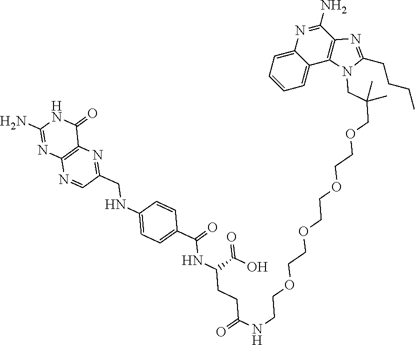

The compounds can be prepared by conventional methods of organic synthesis practiced by those skilled in the art. The general reaction sequences outlined below represent a general method useful for preparing the compounds and are not meant to be limiting in scope or utility.

Descriptions of compounds herein are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art, thereby avoiding inherently unstable compounds.

### Pharmaceutical Compositions

The compounds described herein can be administered alone or formulated as a pharmaceutical composition comprising the compound or compounds and one or more pharmaceutically acceptable excipients. As used herein, the terms “composition” generally refers to any product comprising more than one ingredient, including the compounds described herein. It is to be understood that the compositions described herein can be prepared from isolated compounds or from salts, solutions, hydrates, solvates, and other forms of the compounds. It is appreciated that certain functional groups, such as the hydroxy, amino, and like groups, can form complexes with water and/or various solvents, in the various physical forms of the compounds. It is also to be understood that the compositions can be prepared from various amorphous, non-amorphous, partially crystalline, crystalline, and/or other morphological forms of the compounds, and the compositions can be prepared from various hydrates and/or solvates of the compounds. Accordingly, such pharmaceutical compositions that recite compounds include each of, or any combination of, or individual forms of, the various morphological forms and/or solvate or hydrate forms of the compounds.

One embodiment provides a pharmaceutical composition comprising a compound of Formula (I), (IA), (II), (IIA), (IIB), (III) or (IIIA) or any compound covered by such formulae, or a pharmaceutically acceptable salt thereof, and at least one pharmaceutically acceptable excipient.

One embodiment provides a pharmaceutical composition comprising an effective amount of a therapeutically (or prophylactically) effective compound of Formula (I), (IA), (II), (IIA), (IIB), (III) or (IIIA) or any compound covered by such formulae, or a pharmaceutically acceptable salt thereof, and at least one pharmaceutically acceptable excipient.

Compounds and/or compositions described herein may be administered in unit dosage forms and/or compositions containing one or more pharmaceutically acceptable carriers, adjuvants, diluents, excipients, and/or vehicles, and combinations thereof.

As used herein, the term “administering” generally refers to any and all means of introducing compounds described herein to the host subject including, but not limited to, by oral, intravenous, intramuscular, subcutaneous, transdermal, inhalation, buccal, ocular, sublingual, vaginal, rectal, and like routes of administration.

Administration of the compounds as salts can be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable. Similarly, “pharmaceutically acceptable salt” refers to those salts with counter ions which may be used in pharmaceuticals. Such salts may include, without limitation: (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid, malonic acid, and the like; or (2) salts formed when an acidic proton present in the parent compound either is replaced by a metal ion, e.g., an alkali metal ion, an alkaline earth ion, or an aluminum ion, or coordinates with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Acceptable salts can be obtained using standard procedures known in the art, including (without limitation) reacting a sufficiently acidic compound with a suitable base, affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds described herein are formed from bases that form non-toxic salts. Illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemi-salts of acids and bases, such as hemisulphate and hemicalcium salts, also can be formed.

The compounds can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. Indeed, in at least one embodiment, a compound and/or composition can be administered directly into the blood stream, into muscle, or into an internal organ.

For example, in at least one embodiment, the compounds can be systemically administered (orally, for example) in combination with a pharmaceutically acceptable vehicle, such as an inert diluent or an assimilable edible carrier. For oral therapeutic administration, the active compound can be combined with one or more excipients and used in the form of ingestible tablets, buccal tablets, troches, capsules, elixirs, suspensions, syrups, wafers, and the like. The percentage of the compositions and preparations can vary and may be between about 1 to about 99% weight of the active ingredient(s) and a binder, excipients, a disintegrating agent, a lubricant, and/or a sweetening agent (as are known in the art). The amount of active compound in such therapeutically useful compositions is such that an effective dosage level will be obtained.

The preparation of parenteral compounds/compositions under sterile conditions, for example, by lyophilization, can readily be accomplished using standard pharmaceutical techniques well-known to those skilled in the art. In at least one embodiment, the solubility of a compound used in the preparation of a parenteral composition can be increased by the use of appropriate formulation techniques, such as the incorporation of solubility-enhancing agents.

As previously noted, the compounds/compositions can also be administered via infusion or injection (e.g., using needle (including microneedle) injectors and/or needle-free injectors). Solutions of the active composition can be aqueous, optionally mixed with a nontoxic surfactant and/or contain carriers or excipients, such as salts, carbohydrates and buffering agents (preferably at a pH of from 3 to 9), but, for some applications, they may be more suitably formulated as a sterile non-aqueous solution or as a dried form to be used in conjunction with a suitable vehicle, such as sterile, pyrogen-free water or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents, such as parabens, chlorobutanol, phenol, sorbic acid, thimerosal, and the like. In certain cases, it can be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the active compound and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and freeze-drying, which yield a powder of the active ingredient plus any additional desired ingredient present in the previously sterile-filtered solutions.

For topical administration, it can be desirable to administer the compounds to the skin as compositions or formulations in combination with a dermatologically acceptable carrier, which may be a solid or a liquid. For example, in certain embodiments, solid carriers can include finely divided solids, such as talc, clay, microcrystalline cellulose, silica, alumina and the like. Similarly, useful liquid carriers can comprise water, alcohols or glycols or water-alcohol/glycol blends, in which the compounds can be dissolved or dispersed at effective levels, optionally with the aid of non-toxic surfactants. Additionally or alternatively, adjuvants, such as fragrances and antimicrobial agents, can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject.

Thickeners, such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

As used herein, the term “therapeutically (or prophylactically) effective dose” means (unless specifically stated otherwise) a quantity of a compound which, when administered either one time or over the course of a treatment cycle affects the health, well-being or mortality of a subject (e.g., and without limitation, delays the onset of and/or reduces the severity of one or more of the signs and/or symptoms associated with a fibrotic disease or condition and/or a cancer, as applicable). Useful dosages of the compounds of the present disclosure can be determined by comparing their in vitro activity and the in vivo activity in animal models. Methods of the extrapolation of effective dosages in mice and other animals to human subjects are known in the art. Indeed, the dosage of the compound can vary significantly depending on the condition of the host subject, the cancer or fibrotic disease being treated, how advanced the pathology is, the route of administration of the compound and tissue distribution, and the possibility of co-usage of other therapeutic treatments (such as radiation therapy or additional drugs in combination therapies). The amount of the composition required for use in treatment (e.g., the therapeutically or diagnostically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise. Therapeutically (or prophylactically) effective or diagnostically effective amounts or doses can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. The total therapeutically (or prophylactically) or diagnostically effective amount of the compound can be administered in single or divided doses and can, at the practitioner's discretion, fall outside of the typical range given herein.

In another embodiment, the compound can be administered in a therapeutically (or prophylactically) or diagnostically effective amount of from about 0.5 g/m to about 500 mg/m², from about 0.5 g/m² to about 300 mg/m², or from about 100 g/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². The total amount can be administered in single or divided doses and can, at the physician's discretion, fall outside of the typical range given herein. These amounts are based on m² of body surface area.

### Methods of Treatment

In certain embodiments, the compositions and methods are useful for the prevention and/or treatment of fibrotic diseases. In certain embodiments, the compositions are also useful for the prevention and/or treatment of cancer. The compositions and methods can leverage strategies to (e.g., selectively) target the innate immune system and reprogram the polarization of a macrophage from M2 to M1 and, for example, leverage the antifibrotic properties thereof.

Primarily, there are two main immunity strategies found in vertebrates: the innate immune system and the adaptive immune system. The innate, or non-specific, immune response, is the first line of defense against non-self pathogens and consists of physical, chemical, and cellular defenses. The adaptive immune system, on the other hand, is called into action against pathogens that can evade or overcome the primary innate immune defenses.

Inflammatory response plays a role in immunity. When tissues are damaged or a pathogen is detected, for example, an inflammatory response is initiated, and the immune system is mobilized. The immune cells of the innate immune system (e.g., neutrophils and eosinophils) are the first recruited to the site of tissue injury or damage or pathogen location via blood vessels and the lymphatic system, followed by macrophages.

The cells of the innate immune system can express special pattern recognition receptors that sense and bind with specific protein sequences present in microbial pathogens or other non-self. For example, two classes of molecules that can bind to these pattern recognition receptors are pathogen-associated molecular patterns associated with microbial pathogens and damage-associated molecular patterns associated with components of the host's cells that are released during cell damage or death. Recognition of these protein sequences by the pattern recognition receptors can initiate signal transduction pathways that trigger the expression of certain genes whose products control innate immune responses (e.g., and, eventually (if needed), instruct the development of antigen-specific acquired immunity). Accordingly, the pattern recognition receptors mediate these signaling pathways and can be used to positively or negatively control innate—and even adaptive—immune response.

More specifically, macrophages are a diverse group of white blood cells known for eliminating pathogens through phagocytosis. Macrophages are broadly classified as either having an M1 or M2 phenotype, depending on which specific differentiation they undergo in response to the local tissue environment.

In some instances, macrophages are polarized towards the M1 phenotype by exposure to IFN-γ, lipopolysaccharide (LPS), and/or granulocyte-macrophage colony stimulating factor (GM-CSF). In certain instances, the M1 phenotype is characterized by the production of high levels of pro-inflammatory cytokine(s) (such as IL-10, tumor necrosis factor (TNF), IL-12, IL-18, and/or IL-23), an ability to mediate resistance to pathogens, strong microbicidal properties, high production of reactive nitrogen and oxygen intermediates, and/or promotion of Th1 responses. In some instances, M1 polarization is associated with the “attack and kill” phase of the innate immune response. In certain instances, MI polarization operates to inhibit or prevent initial establishment of infection and/or remove damaged tissue.

In certain instances, after the innate immune system performs this “attack and kill” phase, a macrophage can reprogram itself to become a healing system. In some instances, the macrophage releases growth factors to promote healing. Such growth factors can include (without limitation) certain cytokines, such as IL-4, IL-10, platelet-derived growth factor (PDGF), TGFβ, CCL18, and/or IL-13. In certain instances, exposure to such cytokines/growth factors can alternatively activate the M2 macrophage phenotype.

In contrast to M1, M2 macrophages are typically associated with wound healing and tissue repair. In some instances, M2 macrophages are characterized by their involvement in tissue remodeling, immune regulation/suppression, and/or tumor promotion. In specific instances, M2 macrophages produce polyamines to induce cell proliferation and/or proline to induce collagen production. While this healing response is beneficial in a healthy subject, the presence of M2 macrophages can have significantly detrimental effects through immune suppression and/or the promotion of tumor growth and fibrosis for those subjects suffering from a fibrotic disease or cancer.

For example, fibrotic pathologies can begin with an unknown trauma or insult to the epithelium. In response to the resulting tissue damage, chemokines and other factors can be released to promote the infiltration of immune cells to the damaged tissue (e.g., an innate immune response), which, for example, include monocytes and macrophages that assume an M2-like phenotypes and, for example, release profibrotic cytokines. The chronic secretion of these cytokines can then activate tissue-resident and infiltrating fibroblasts/fibrocytes to become myofibroblasts that, in turn, secret collagen and other extracellular matrix proteins that can stiffen the surrounding tissue. In this way, these M2 macrophages can exacerbate the disease and act profibrotic. For example, M2 macrophages can infiltrate the lungs of idiopathic pulmonary fibrosis (IPF) subjects, for example, and promote fibrosis therein, which facilitates progression of the disease.

The growth factors and other cytokines produced by the M2 phenotype can drive cancerous tumor growth through similar pathways. For example, cancers can also involve an anti-inflammatory immune response that promotes the growth of cancerous tumors (e.g., owing to the growth factors secreted by the activated M2-like macrophages, specifically tumor associated macrophages (TAMs)) and/or promotes collagen formation in cancerous tumors (e.g., through downstream fibrotic collagen production). In certain instances, this can result in a cancerous tumor that is more advanced and more difficult to treat because its growth decreases the drug penetrability thereof.

Reprogramming M2-like Macrophages to M1-like Macrophages

In certain cancers and fibrotic diseases, macrophages are disproportionately biased towards the anti-inflammatory pro-fibrotic (M2-like) phenotype. In certain instances, immune modulators (e.g., TLR7 agonists) can convert—e.g., reprogram—activated myeloid cells (e.g., M2-like macrophages) into a pro-inflammatory and antifibrotic M1 polarization (e.g., where they produce little or no growth factors and/or related cytokines and, for example, slow or even eliminate the progression of the disease state). Provided herein are compounds, compositions and methods that reverse the antifibrotic/proinflammatory to profibrotic/anti-inflammatory (M1>M2) macrophage shift observed during the course of the development of fibrotic diseases (such as, for example, IPF) and certain cancers. In some embodiments, the compounds, compositions and methods described herein decrease the amount of pro-fibrotic/anti-inflammatory biomarkers (e.g., of profibrotic activity (for example, CCL18, hydroxyproline, and collagen)) in an individual or a sample thereof. In some embodiments, the compounds, compositions and methods described herein increase anti-fibrotic/proinflammatory biomarkers (for example, TNFα and IFN-γ). In some embodiments, the compounds, compositions and methods described herein reprogram M2-like macrophages into M1-like macrophages. In some embodiments, the compounds, compositions and methods described herein alter the cytokine secretions and chemoattractants generated by macrophages. In some embodiments, provided are compositions that reverse the M2-like phenotypic shift to provide an effective treatment for fibrotic diseases, disorders, or conditions thereof, as well as for certain types of cancer.

In at least one embodiment, a drug comprising an immune modulator (e.g., a TLR7 agonist) is used to make the compounds and compositions used in the methods. Any therapeutic agent (e.g, drug) suitable for reprogramming activated macrophages (M2-like phenotype) to an M1-like phenotype (e.g., a TLR7 agonist) can be used and the drug (or warhead) can operate in the endosome and/or cytoplasm of the cell (e.g., depending on its structure). In at least one embodiment, the therapeutic agent (e.g., drug) is an immune modulator (e.g., that positively controls a pattern recognition receptor and/or its downstream signaling pathways (in each case, part of the innate immune system)), such as, for example, a TLR agonist. In some embodiments, the drug is a TLR7 agonist according to any one of Formula (I), (IA), (II), (IIA), (IIB), (III) or (IIIA).

In certain embodiments, a compound is provided that comprises a targeting moiety (or a radical thereof; for example, folate) attached to an immune modulator (or a radical thereof; for example, a TLR7 agonist) that targets a pattern recognition receptor of a cell, the targeting moiety comprising a folate ligand or a functional fragment or analog thereof. In some embodiments, the immune modulator is a TLR7 agonist.

One embodiment provides a method for treating a cancer in an individual in need thereof. The method comprises administering a therapeutically effective amount of one or more compounds of any one of Formula (I), (IA), (II), (IIA), (IIB), (III) or (IIIA), any compound covered by such formulae, or a composition comprising same to the individual in need thereof. In some embodiments, the cancer is characterized by a tumor comprising TAMs. In some embodiments, the cancer is selected from the group consisting of lung cancer, bone cancer, pancreatic cancer, skin cancer, cancer of the head, cancer of the neck, cutaneous melanoma, intraocular melanoma, uterine cancer, ovarian cancer, endometrial cancer, epithelial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, bladder cancer, gastric cancer, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer, and adenocarcinoma of the gastroesophageal junction. In certain embodiments, the cancer is lung cancer, breast cancer, colon cancer, ovarian cancer, pancreatic cancer or epithelial cancer.

In one embodiment, the cancer is lung cancer.

In one embodiment, the cancer lung cancer, triple negative breast cancer, colon cancer, gastric cancer, bladder cancer, prostate cancer, or pancreatic cancer.

One embodiment provides a method for treating an inflammatory disease or disorder. The method comprises administering a therapeutically effective amount of one or more compounds of any one of Formula (I), (IA), (II), (IIA), (IIB), (III) or (IIIA), any compound covered by such formulae, or a composition comprising same to a patient in need thereof.

One embodiment provides a method for treating a fibrotic disease or disorder in an individual in need thereof. The method comprises administering a therapeutically effective amount of one or more compounds of any one of Formula (I), (IA), (II), (IIA), (IIB), (III) or (IIIA), any compound covered by such formulae, or a composition comprising same to the individual in need thereof.

In certain embodiments, the fibrotic disease or disorder is selected from the group consisting of arthrofibrosis, autoimmune pancreatitis, bladder fibrosis, chronic kidney disease, chronic wounds, Crohn's disease, desmoid tumor, Dupuytren's contracture, endometrial fibroids, fibromatosis, graft-versus-host disease (GVHD), heart fibrosis, keloids, liver fibrosis (e.g., nonalcoholic steatohepatitis (NASH) or cirrhosis), mediastinal fibrosis, myelofibrosis, nephrogenic systemic fibrosis, Peyronie's disease, pulmonary fibrosis, retroperitoneal cavity fibrosis, scleroderma or systemic sclerosis, and skin fibrosis.

In certain embodiments, the fibrotic disease or disorder is idiopathic pulmonary fibrosis (IPF), liver fibrosis, myelofibrosis, or cardiac fibrosis.

In other embodiments, the fibrotic disease or disorder or inflammatory disease or disorder is selected from the group consisting of lupus, inflammatory bowel disease (IBS), Addison's disease, Grave's disease, Sjogren's syndrome, celiac disease, Hashimoto's thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, transplant rejection, fatty liver disease, asthma, osteoporosis, sarcoidosis, ischemia-reperfusion injury, prosthesis osteolysis, glomerulonephritis, scleroderma, psoriasis, with autoimmune myocarditis, spinal cord injury, central nervous system, viral infection, influenza, coronavirus infection, cytokine storm syndrome, bone damage, inflammatory brain disease, and atherosclerosis.

One embodiment provides a method for inhibiting or reducing an inflammatory disease or disorder. The method comprises administering a compound of Formula (I), (IA), (II), (IIA), (IIB), (III) or (IIIA), any compound covered by such formulae, or a composition comprising same to a patient in need thereof.

One embodiment provides a compound of Formula (I), (IA), (II), (IIA), (IIB), (III) or (IIIA), any compound covered by such formulae, or a composition comprising such compound, for use in a method for treating cancer (e.g., any of the types of cancer listed herein).

One embodiment provides a compound of Formula (I), (IA), (II), (IIA), (IIB), (III) or (IIIA), any compound covered by such formulae, or a composition comprising such compound, for use in a method for treating an inflammatory disease or disorder. In some embodiments, the inflammatory disease or disorder is a fibrotic disease or disorder. In certain embodiments, the fibrotic disease or disorder is IPF, liver fibrosis, myelofibrosis, or cardiac fibrosis.

One embodiment provides a compound of Formula (I), (IA), (II), (IIA), (IIB), (III) or (IIIA), any compound covered by such formulae, or a composition comprising such compound, for use in a method for inhibiting or reducing cancer.

One embodiment provides a compound of Formula (I), (IA), (II), (IIA), (IIB), (III) or (IIIA), any compound covered by such formulae, or a composition comprising such compound, for use in a method for inhibiting or reducing fibrosis.

One embodiment provides the use of a compound of Formula (I), (IA), (II), (IIA), (IIB), (III) or (IIIA), any compound covered by such formulae, or a composition comprising such compound, for the manufacturing of a medicament for treating cancer.

One embodiment provides the use of a compound of Formula (I), (IA), (II), (IIA), (IIB), (III) or (IIIA), any compound covered by such formulae, or a composition comprising such compound, for use in a method for the manufacture of a medicament for treating an inflammatory disease. In some embodiments, the inflammatory disease or disorder is a fibrotic disease or disorder.

A method for inhibiting or reducing fibrosis (e.g., in an individual in need thereof, such as an individual suffering from a cancer or a fibrotic disease) is provided, such method comprising administering (e.g., to the individual) one or more compounds of Formula (I), (IA), (II), (IIA), (IIB), (III) or (IIIA) or any compound(s) covered by such formulae, or a pharmaceutically acceptable salt thereof, or a pharmaceutical composition comprising the same, to an individual in need thereof in an amount effective to convert a population of macrophages biased towards an M2-like phenotype (e.g., profibrotic/anti-inflammatory) to an M1-like phenotype (e.g., antifibrotic/proinflammatory), wherein the population of macrophages are present in a targeted location within the individual, the M2-like phenotype is associated with an anti-inflammatory/profibrotic state, and the M1-like phenotype is associated with a proinflammatory/antifibrotic state.

In addition, a method for inhibiting or reducing cancerous growth (e.g., in an individual in need thereof, such as an individual suffering from cancer) is provided, such method comprising administering (e.g., to the individual) one or more compounds of Formula (I), (IA), (II), (IIA), (IIB), (III) or (IIIA) or any compound(s) covered by such formulae, or a pharmaceutically acceptable salt thereof, or a pharmaceutical composition comprising the same, to an individual in need thereof in an amount effective to convert a population of macrophages biased towards an M2-like phenotype (e.g., profibrotic/anti-inflammatory) to an M1-like phenotype (e.g., antifibrotic/proinflammatory), wherein the population of macrophages are present in a targeted location within the individual, the M2-like phenotype is associated with an anti-inflammatory/profibrotic state, and the M1-like phenotype is associated with a proinflammatory/antifibrotic state. In at least one embodiment, the targeted location is a tumor microenvironment.

One embodiment provides the use of a compound of Formula (I), (IA), (II), (IIA), (IIB), (III) or (IIIA), any compound covered by such formulae, or a composition comprising such compound, for the manufacturing of a medicament for treating a cancer.

Provided herein, in certain embodiments, are methods of treating lung cancer in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the lung cancer. In some embodiments, the lung cancer comprises a tumor comprising TAMs in M2 form (e.g., M2-like phenotype). In some embodiments, administering a compound of Formula (I), (IA), (II), (IIA), (IIB), (III) or (IIIA), any compound covered by such formulae, or a composition comprising such compound reprograms an M2 macrophage into M1 phenotype.

Provided herein, in certain embodiments, are methods of treating triple negative breast cancer in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the triple negative breast cancer. In some embodiments, the triple negative breast cancer comprises a tumor comprising TAMs in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating colon cancer in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the colon cancer. In some embodiments, the colon cancer comprises a tumor comprising TAMs in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating gastric cancer in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the gastric cancer. In some embodiments, the gastric cancer comprises a tumor comprising TAMs in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating prostate cancer in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the prostate cancer. In some embodiments, the prostate cancer comprises a tumor comprising TAMs in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating bladder cancer in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the bladder cancer. In some embodiments, the bladder cancer comprises a tumor comprising TAMs in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating pancreatic cancer in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the pancreatic cancer. In some embodiments, the pancreatic cancer comprises a tumor comprising TAMs in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

One embodiment provides the use of a compound of Formula (I), (IA), (II), (IIA), (IIB), (III) or (IIIA), any compound covered by such formulae, or a composition comprising such compound, for the manufacturing of a medicament for inhibiting or reducing fibrosis.

In one embodiment, the fibrosis is pulmonary fibrosis (e.g., IPF), liver fibrosis, or cardiac fibrosis. In some embodiments, the fibrosis is selected from a group consisting of fatty liver disease, cirrhosis, colitis, chronic liver disease, cardiac fibrosis, and scleroderma. In certain embodiments, the fibrotic disease or disorder is pulmonary fibrosis, liver fibrosis, scleroderma, myelofibrosis, Crohn's disease, or chronic kidney disease.

Provided herein, in certain embodiments, are methods of treating pulmonary fibrosis in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the pulmonary fibrosis. In some embodiments, the pulmonary fibrosis comprises pro-fibrotic macrophages in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating liver fibrosis in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the liver fibrosis. In some embodiments, the liver fibrosis comprises pro-fibrotic macrophages in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating scleroderma in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the scleroderma. In some embodiments, the scleroderma comprises pro-fibrotic macrophages in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating myelofibrosis in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the myelofibrosis. In some embodiments, the myelofibrosis comprises pro-fibrotic macrophages in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating Crohn's disease in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the Crohn's disease. In some embodiments, the Crohn's disease comprises pro-fibrotic macrophages in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

Provided herein, in certain embodiments, are methods of treating chronic kidney disease in an individual in need thereof, comprising administering to that individual a therapeutically effective amount of a compound of Formula (I), (IA), (II), (IIA), (IIB), (III) or (IIIA), any compound covered by such formulae, or a composition comprising such compound, thereby treating the chronic kidney disease. In some embodiments, the chronic kidney disease comprises pro-fibrotic macrophages in M2 form. In some embodiments, administering a compound of Formula (I), (IA), (II), (IIA), (IIB), (III) or (IIIA), any compound covered by such formulae, or a composition comprising such compound reprograms an M2 macrophage into M1 form.

In some embodiments of any of the methods of treating a cancer or a fibrotic disease or disorder disclosed herein, the method (e.g., administration of the one or more compounds) does not induce unwanted inflammation in the individual.

In some embodiments of any of the methods of treating a cancer or a fibrotic disease or disorder disclosed herein, the method further comprises administering a second therapeutic agent. In some embodiments, the second therapeutic agent is an anti-inflammatory agent. In some embodiments, the second therapeutic agent is a proinflammatory agent (e.g., if the method is for treating a fibrotic disease or disorder). In some embodiments, the second therapeutic agent is a chemotherapeutic agent (e.g., if the method is for treating a cancer). In some embodiments, the compounds or compositions of the disclosure are administered in combination with the second therapeutic agent simultaneously or sequentially.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

While certain embodiments of the present disclosure have been shown and described herein, it will be apparent to those skilled in the art that such embodiments are provided by way of example only. It is not intended that the claimed invention be limited by the specific examples provided within the specification.

While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein, which depend upon a variety of conditions and variables. It should be understood that various alternatives to the embodiments of the invention described herein may be employed in practicing the invention. It is, therefore, contemplated that the invention shall also cover any such alternatives, modifications, variations or equivalents. It is intended that the following claims define the scope of the invention and that methods and structures within the scope of these claims and their equivalents be covered thereby.

### Certain Definitions

As used herein and in the appended claims, the singular forms “a,” “and,” and “the” include plural referents unless the context clearly dictates otherwise. Thus, for example, reference to “a compound” includes a plurality of such compounds. When ranges are used herein for physical properties, such as molecular weight, or chemical properties, such as chemical formulae, all combinations and sub-combinations of ranges and specific embodiments therein are intended to be included. The term “about,” when referring to a number or a numerical range, means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude an embodiment of any compound, composition, method, process, or the like that may “consist of” or “consist essentially of” the described features.

The terms “treat,” “treating,” or “treatment” include reducing, alleviating, abating, ameliorating, relieving, or lessening the symptoms associated with a cancer, fibrotic disease or disorder, or inflammatory conditions or diseases in either a chronic or acute therapeutic scenario. In some embodiments, treatment of a fibrotic disease or disorder includes reducing fibrosis. In some embodiments, treatment of a cancer includes reducing the number of M2-like macrophages found in an associated tumor.

As used herein, the terms “patient,” “subject,” and “individual” are used interchangeably. None of the terms require the supervision of medical personnel. For example, administering to an individual includes the individual administering the therapeutic agent to themselves, as well as a medical professional administering the therapeutic agent to the individual.

“Alicyclic” refers to a radical in which there is at least one all-carbon ring which may be fully or partially saturated, and optionally there may be one or more straight chain groups attached. For example, cycloalkyl and cycloalkenyl groups such as cyclobutyl and cyclohex-3-enyl would be considered alicyclic, as would cycloalkyl groups with one or more straight chain alkyl groups attached, for example cyclohexylmethyl, 3-n-propylcyclopent-2-enylmethyl, or 2,3,4-trimethylcyclohexyl. The attachment point of the alicyclic radical may be on either a ring or chain atom.

“Alkyl” refers to a straight or branched or cyclic hydrocarbon chain radical consisting solely of carbon and hydrogen atoms, containing no unsaturation, and having from one to fifteen carbon atoms (e.g., C₁-C₁₅ alkyl). In various embodiments, an alkyl comprises three to six carbon atoms (e.g., C₃-C₆ alkyl), one to thirteen carbon atoms (e.g., C₁-C₁₃ alkyl), one to eight carbon atoms (e.g., C₁-C₈ alkyl), one to five carbon atoms (e.g., C₁-C₈ alkyl), one to four carbon atoms (e.g., C₁-C₄ alkyl), one to three carbon atoms (e.g., C₁-C₃ alkyl), one to two carbon atoms (e.g., C₁-C₂ alkyl), one carbon atom (e.g., C₁ alkyl), five to fifteen carbon atoms (e.g., C₅-C₁₅ alkyl), five to eight carbon atoms (e.g., C₅-C₈ alkyl), two to five carbon atoms (e.g., C₂-C₅ alkyl), or three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

The alkyl group can comprise a carbocyclic or carbocyclyl group. “Carbocyclyl” refers to a stable, non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})², —R^{b}—N(R^{a})², —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Alkoxyl” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group is through one carbon in the alkylene chain or through any two carbons within the chain. In certain embodiments, an alkylene comprises one to eight carbon atoms (e.g., C₁-C₈ alkylene), one to seven carbon atoms (e.g., C₁-C₇ alkylene), one to six carbon atoms (e.g., C₁-C₆ alkylene), one to five carbon atoms (e.g., C₁-C₅ alkylene), one to four carbon atoms (e.g., C₁-C₄ alkylene), one to three carbon atoms (e.g., C₁-C₃ alkylene), or one to two carbon atoms (e.g., C₁-C₂ alkylene). In other embodiments, an alkylene comprises one carbon atom (e.g., C₁ alkylene), five to eight carbon atoms (e.g., C₅-C₈alkylene), two to five carbon atoms (e.g., C₂-C₅ alkylene), or three to five carbon atoms (e.g., C₃-C₅ alkylene). Unless specifically stated otherwise, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene), two to five carbon atoms (e.g., C₂-C₈ alkenylene), two to four carbon atoms (e.g., C₂-C₄ alkenylene), or two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene) or three to five carbon atoms (e.g., C₃-C₅ alkenylene). Unless specifically stated otherwise, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene), two to five carbon atoms (e.g., C₂-C₈ alkynylene), two to four carbon atoms (e.g., C₂-C₄ alkynylene), two to three carbon atoms (e.g., C₂-C₃ alkynylene), or two carbon atoms (e.g., C₂ alkylene). In other embodiments, an alkynylene comprises five to eight carbon atoms (e.g., C₅-C₈ alkynylene) or three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Huckel theory. The ring system from which aryl groups are derived include, but are not limited to, groups such as benzene, fluorene, indane, indene, tetralin and naphthalene. Unless specifically stated otherwise, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})², —R^{b}—N(R^{a})², —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above (e.g., methylene, ethylene, and the like). The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where Rd is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —Re-aryl, where Re is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above (e.g., methylene, ethylene, and the like). The alkylene chain part of the aralkyl radical is optionally substituted as described above for an alkylene chain. The aryl part of the aralkyl radical is optionally substituted as described above for an aryl group.

“Biaryl” refers to a radical of the formula —Ar—Ar, wherein two aryl groups are joined by a single bond. Biphenyl is an example of a biaryl radical.

“Carbocyclylalkyl” refers to a radical of the formula —R^{c}-carbocyclyl, where R^{c} is an alkylene chain as defined above. The alkylene chain and the carbocyclyl radical are optionally substituted as defined above.

“Carbocyclylalkenyl” refers to a radical of the formula —R^{c}-carbocyclyl, where R^{c} is an alkenylene chain as defined above. The alkenylene chain and the carbocyclyl radical are optionally substituted as defined above.

“Carbocyclylalkynyl” refers to a radical of the formula —R^{c}-carbocyclyl, where R^{c} is an alkynylene chain as defined above. The alkynylene chain and the carbocyclyl radical are optionally substituted as defined above.

“Carbocyclylalkoxy” refers to a radical bonded through an oxygen atom of the formula —O—Rc-carbocyclyl, where Rc is an alkylene chain as defined above. The alkylene chain and the carbocyclyl radical are optionally substituted as defined above.

“Halo” or “halogen” refers to bromo, chloro, fluoro or iodo substituents.

“Cyano” refers to the group —CN.

“Oxo” refers to the group ═O.

“Haloalkyl” refers to an alkyl radical with one or more halogen substituents. For example, haloalkyl includes groups such as trifluoromethyl, 3-fluoro-2-chloropropyl, and 4-bromocyclohexyl.

“Haloalkylenyl” refers to an alkenyl radical with one or more halogen substituents. For example, haloalkenyl includes groups such as 2,2-difluorovinyl, and 4-chloro-2-butenyl.

“Heteroalkyl” refers to a radical of a saturated straight or branched alkyl chain wherein at least one carbon atom in the chain is replaced with a heteroatom, such as O, S, or N. In some embodiments, a heteroalkyl group may comprise, e.g., 1-12, 1-10, or 1-6 carbon atoms, referred to herein as C₁-C₁₂ heteroalkyl, C₁-C₁₀ heteroalkyl, and C₁-C₆ heteroalkyl. In certain instances, a heteroalkyl group comprises 1, 2, 3, or 4 independently selected heteroatoms in place of 1, 2, 3, or 4 individual carbon atoms in the alkyl chain. Representative heteroalkyl groups include, for example, CH₂CH₂OCH₃, —CH₂CH₂NHCH₃, —CH₂CH₂N(CH₃)CH₃, and the like.

“Heterocyclyl” refers to a stable 3- to 18-membered non-aromatic ring radical that comprises two to twelve carbon atoms and from one to six heteroatoms selected from nitrogen, oxygen and sulfur. Unless specifically stated otherwise, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless specifically stated otherwise, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})², —R^{b}—N(R^{a})², —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“Heterocyclylalkyl” refers to a radical of the formula —R^{c}-heterocyclyl, where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl, where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Huckel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless specifically stated otherwise, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})², —R^{b}—N(R^{a})², —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

“Pharmacokinetic” properties refer to properties of what a living body (e.g., the body of a subject to which a compound has been administered) does to a compound. These properties include absorption, bioavailability, distribution, metabolism, and elimination. Improvement of these properties refers to change to values that are more desirable for the drug properties of the administered compound. For example, higher bioavailability is usually considered an improvement in a pharmacokinetic property.

“Pharmacodynamic” properties refers to properties of what a compound does to a living body (e.g., the body of a subject to which a compound has been administered). These properties can include receptor binding, agonism, antagonism, interaction with carrier proteins, and the like. Improvement of these properties refers to change to values that are more desirable for the drug properties of the administered compound. For example, more potent binding to a target receptor is usually considered an improvement in a pharmacokinetic property.

As described herein, certain compounds of the present disclosure can contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group can have a suitable substituent at each substitutable position of the group, and when more than one position in any given structure may be substituted with more than one substituent selected from a specified group, the substituent can be either the same or different at each position. Combinations of substituents envisioned are preferably those that result in the formation of stable or chemically feasible compounds.

The term “stable”, as used herein, refers to compounds that are not substantially altered when subjected to conditions to allow for their production, detection, and, in certain embodiments, their recovery, purification, and use for one or more of the purposes disclosed herein.

Suitable substituents for an optionally substituted alkyl, alkylene, haloalkyl, haloalkylene, heteroalkyl, heteroalkylene, carbocyclyl, heterocyclyl, aryl group and heteroaryl group include but are not limited to halogen, ═O, CN, —OR^{c}, —NR^{d}R^{e}, —S(O)ₖR^{c}, —NR^{c}S(O)₂R^{c}, —S(O)₂NR^{d}R^{e}, —C(═O)OR^{c}, —OC(═O)OR^{c}, —OC(═O)R^{c}, OC(═S)OR^{c}, —C(═S)OR^{c}, —O(C═S)R^{c}, —C(═O)NR^{d}R^{e}, —NR^{c}C(═O)R^{c}, —C(═S)NR^{d}R^{e}, —NR^{c}C(═S)R^{c}, —NR^{c}(C═O)OR^{c}, —O(C═O)NR^{d}R^{e}, —NR^{c}(C═S)OR^{c}, —O(C═S)NR^{d}R^{e}, —NR^{c}(C═O)NR^{d}R^{e}, —NR^{c}(C═S)NR^{d}R^{e}, —C(═S)R^{c}, —C(═O)R^{c}, C₁-C₆ alkyl, C₁-C₆ haloalkyl, C₁-C₆ heteroalkyl, carbocyclyl, (C₁-C₆-alkylene)-carbocyclyl, (C₁-C₆-heteroalkylene)-carbocyclyl, heterocyclyl, (C₁-C₆-alkylene)-heterocyclyl, (C₁-C₆-heteroalkylene)-heterocyclyl, aryl, (C₁-C₆-alkylene)-aryl, (C₁-C₆-heteroalkylene)-aryl, heteroaryl, (C₁-C₆-alkylene)-heteroaryl, or (C₁-C₆-heteroalkylene)-heteroaryl, wherein each of said alkyl, alkylene, heteroalkyl, heteroalkylene, carbocyclyl, heterocyclyl, aryl and heteroaryl are optionally substituted with one or more of halogen, OR^{c}, —NO₂, —CN, —NR^{c}C(═O)R^{e}, —NR^{d}R^{e}, —S(O)ₖR^{c}, —C(═O)OR^{c}, —C(═O)NR^{d}R^{e}, —C(═O)R^{c}, C₁-C₆ alkyl, C₁-C₆ haloalkyl, or C₁-C₆ heteroalkyl, and wherein R^{c} is hydrogen, hydroxy, C₁-C₆ alkyl, C₁-C₆ heteroalkyl, carbocyclyl, (C₁-C₆-alkylene)-carbocyclyl, (C₁-C₆-heteroalkylene)-carbocyclyl, heterocyclyl, (C₁-C₆-alkylene)-heterocyclyl, (C₁-C₆-heteroalkylene)-heterocyclyl, aryl, (C₁-C₆-alkylene)-aryl, (C₁-C₆-heteroalkylene)-aryl, heteroaryl, (C₁-C₆-alkylene)-heteroaryl, or (C₁-C₆-heteroalkylene)-heteroaryl, each of which is optionally substituted with one or more of halogen, hydroxy, C₁-C₆ alkyl, C₁-C₆ haloalkyl, C₁-C₆ heteroalkyl, carbocyclyl, heterocyclyl, aryl, or heteroaryl; R^{d} and R^{e} are each independently selected from hydrogen, C₁-C₆ alkyl, or C₁-C₆ heteroalkyl; and k is 0, 1 or 2.

The term “radical” as used herein refers to a fragment of a molecule, wherein that fragment has an open valence which is an attachment point for bond formation. A monovalent radical has one open valence such that it can form one bond with another chemical group. In some embodiments, a radical of a molecule (e.g., a radical of a folate receptor binder) as used herein is created by removal of one hydrogen atom from that molecule to create a monovalent radical with one open valence at the location where the hydrogen atom was removed. Where appropriate, a radical can be divalent, trivalent, etc., wherein two, three or more hydrogen atoms have been removed to create a radical which can bond to two, three, or more chemical groups. Where appropriate, a radical open valence can be created by removal of other than a hydrogen atom (e.g., a halogen atom), or by removal of two or more atoms (e.g., a hydroxyl group), as long as the atoms removed are a small fraction (about 20% or less of the atom count) of the total atoms in the molecule forming the radical.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans). Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

### EXAMPLES

All solvents, reagents and starting materials were purchased from commercial vendors and used without further purification, unless otherwise described, or synthesized according to the methods described herein or using literature procedures.

Cysteine Wang resin was obtained from Novabiochem (San Diego, Calif.). Amino acids were purchased from Chem-Impex International (Chicago, Ill.). All reagents used for synthesis were purchased from Sigma-Aldrich (St. Louis, Mo.). Bromo PEG compounds were purchased from Broadpharm (San Diego, Calif.). All other cell culture reagents, syringes, and disposable items were purchased from VWR (Chicago, Ill.).

All reactions were performed at room temperature unless otherwise stated.

NMR spectra were recorded on a 500 MHz Bruker AV500HD Spectrometer. All preparative high performance liquid chromatography (HPLC) was performed with an Agilent 1200 Instrument with a reverse-phase XBridge OBD preparative column (19×150 mm, 5 μm) manufactured by Waters (Milford, Mass.) with UV detection at 254 nm. Low-resolution mass spectrometry-liquid chromatography/mass spectrometry (LRMS-LC/MS) was performed on an Agilent 1220 Infinity LC with a reverse-phase XBridge Shield RP18 column (3.0×50 mm, 3.5 μm). CombiFlash column chromatography was used to purify the compounds. The purity of all final compounds was ≥95% as determined by analytical HPLC on a reverse-phase column with the binary system ammonium acetate (20 mM, pH—7) and acetonitrile as eluent.

### Example 1: Synthesis of TLR7 Agonist (Compound 1)

Compound 1 was synthesized according to Scheme 2 below:

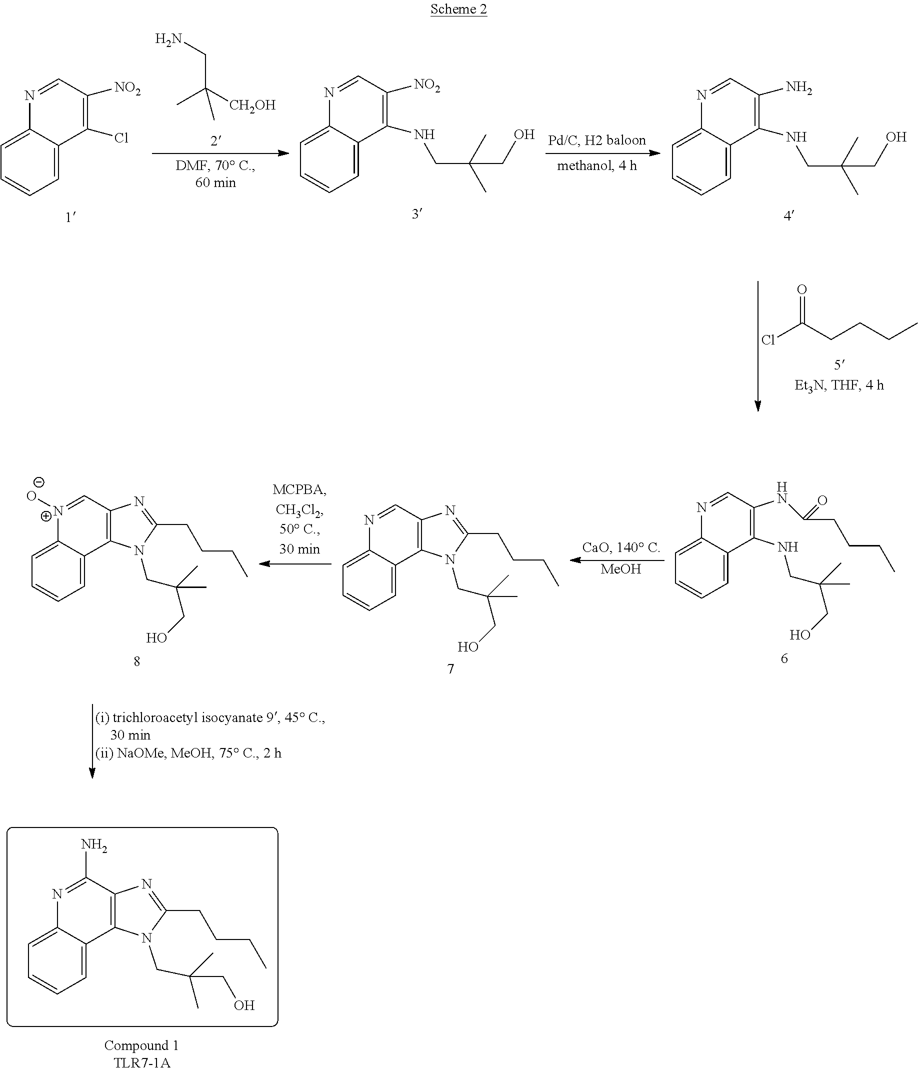

### Step 1: Synthesis of 2,2-dimethyl-3-((3-nitroquinolin-4-yl)amino)propan-1-ol

To a stirred solution of 4-chloro-3-nitroquinoline (1.2 equiv) (Compound 1′ in scheme 2) in N,N-dimethyl formamide (10 mL), 3-amino-2,2-dimethylpropan-1-ol (1.5 equiv) (Compound 2′ in Scheme 2) and triethyl amine (2 equiv) were added. The reaction mixture was heated at 70° C. for 60 min and monitored through liquid chromatography-mass spectrometry (LCMS). It was then cooled down, diluted with water, and stirred for another 15 minutes. The precipitated solid was filtered and washed with water. The solid was dried under vacuum to get material (Compound 3′ in Scheme 2) as yellow solid. Yield was 80%.

### Step 2: Synthesis of 3-((3-aminoquinolin-4-yl)amino)-2,2-dimethylpropan-1-ol

2,2-dimethyl-3-(3-nitroquinolin-4-ylamino)propan-1-ol (1 g) (Compound 3′ in Scheme 2) was dissolved in methanol (15 mL) and reduced over Pd/C (100 mg, 10 mol %) as catalyst under hydrogen balloon condition for 4 hours. The solution was then filtered and evaporated under reduced pressure to get 3-(3-aminoquinolin-4-ylamino)-2,2-dimethylpropan-1-ol (Compound 4′ in scheme 2).

### Step 3: Synthesis of N-(4-((3-hydroxy-2,2-dimethylpropyl)amino)quinoline-3-yl)pentanamide

Triethylamine (2 equiv) and valeryl chloride (Compound 5′ in Scheme 2, 1.5 equiv) were added to a stirred solution of Compound 4′ (1 g) in anhydrous tetrahydrofuran (THF) (10 mL). The reaction mixture was then stirred for 4 hours, followed by removal of the solvent under reduced pressure. The crude residue was dissolved in ethyl acetate (EtOAc), washed with water and brine and dried over sodium sulphate. The combined organic layer was evaporated to dryness under vacuum to obtain the intermediate amide Compound 6 (see Scheme 2). Overall yield was 70%.

### Step 4: Synthesis of 3-(2-butyl-1H-imidazo[4,5-c]quinoline-1-yl)-2,2-dimethylpropan-1-ol

To a stirred solution of the amide Compound 6 (1 g) in MeOH (15 mL) was added excess calcium oxide (10 equiv) and the solution was heated at 110° C. for 96 hours. The solvent was then removed under vacuum and the residue was purified using flash column chromatography (MeOH/dichloromethane mobile phase) to obtain Compound 7 of Scheme 2. Yield was 60%.

### Step 5: Synthesis of 3-(4-amino-2-butyl-1H-imidazo[4,5-c]quinolin-1-yl)-2,2-dimethylpropan-1-ol (Compound 1)

3-chloroperoxybenzoic acid (1.5 equiv) was added to a stirred solution of Compound 7 of scheme 2 (100 mg) in anhydrous dichloromethane (1 mL), and the solution was refluxed at 50° C. for 30 minutes. Once the starting material was completely consumed, the solvent was evaporated to dryness under vacuum.

The residue was then re-dissolved in anhydrous dichloromethane (1 mL), followed by the addition of trichloroacetyl isocyanate (2.0 equiv) (see Compound 9′ in Scheme 2) and the reaction mixture was heated at 45° C. for 30 minutes. After the completion of the reaction, the solvent was removed under vacuum and the residue was re-dissolved in anhydrous MeOH (1 mL), followed by the addition of 25% methanolic sodium methoxide solution (0.2 mL). This was then heated at 75° C. for an hour and cooled down. The solvent was removed under vacuum and the residue was purified using column chromatography (MeOH/dichloromethane) to obtain compound 1 (TLR7-1A) as a colorless liquid. Overall yield was 70%. LCMS: [M+H]+ m/z=327.

### Example 2: Synthesis of TLR7 Agonist (Compound 2)

Compound 2 was synthesized according to Scheme 3 below:

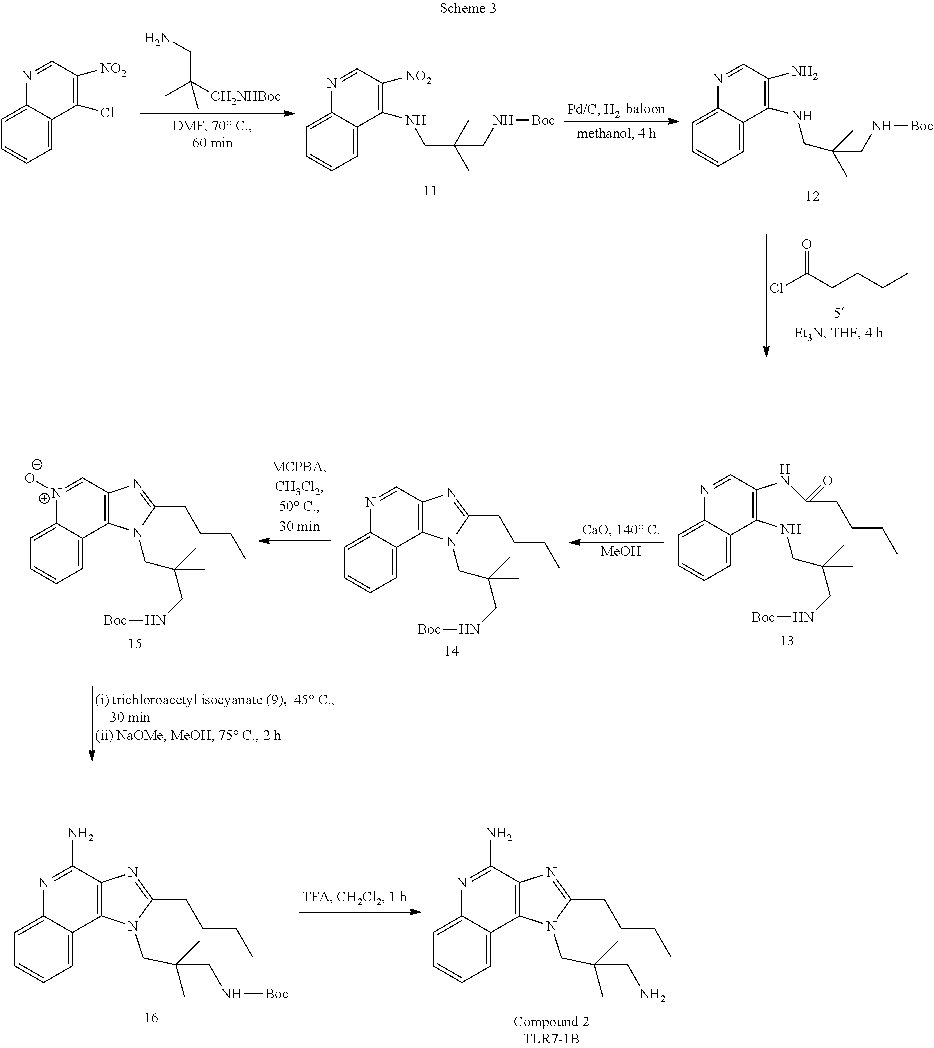

Synthetic procedure to prepare tert-butyl (3-(4-amino-2-butyl-1H-imidazo[4,5-c]quinolin-1-yl)-2,2-dimethylpropyl)carbamate (Compound 16 in Scheme 3) follows the above discussed procedure using tert-butyl (3-amino-2,2-dimethylpropyl)carbamate as the starting material.

### Boc-Deprotection of tert-butyl (3-(4-amino-2-butyl-1H-imidazo[4,5-c]quinolin-1-yl)-2,2-dimethylpropyl)carbamate

To a stirred solution of tert-butyl (3-(4-amino-2-butyl-1H-imidazo[4,5-c]quinolin-1-yl)-2,2-dimethylpropyl)carbamate (100 mg, 1 equiv) (Compound 16 in Scheme 3) in 1,2-dichloromethane (1 mL) was added trifluoroacetic acid (10 equiv). This was stirred under nitrogen atmosphere for 1 h. Once the starting materials were completely consumed as determined by thin-layer chromatography (TLC), it was evaporated to dryness under vacuum and basified with saturated sodium bicarbonate. This was then purified using column chromatography (MeOH/dichloromethane) to obtain 1-(3-amino-2,2-dimethylpropyl)-2-butyl-1H-imidazo[4,5-c]quinolin-4-amine (Compound 2—TLR7-1B) as a colorless liquid. LCMS: [M+H]+ m/z=326.

### Example 3: Synthesis of TLR7 Agonist (Compound 3)

Compound 3 was synthesized according to Scheme 4 below:

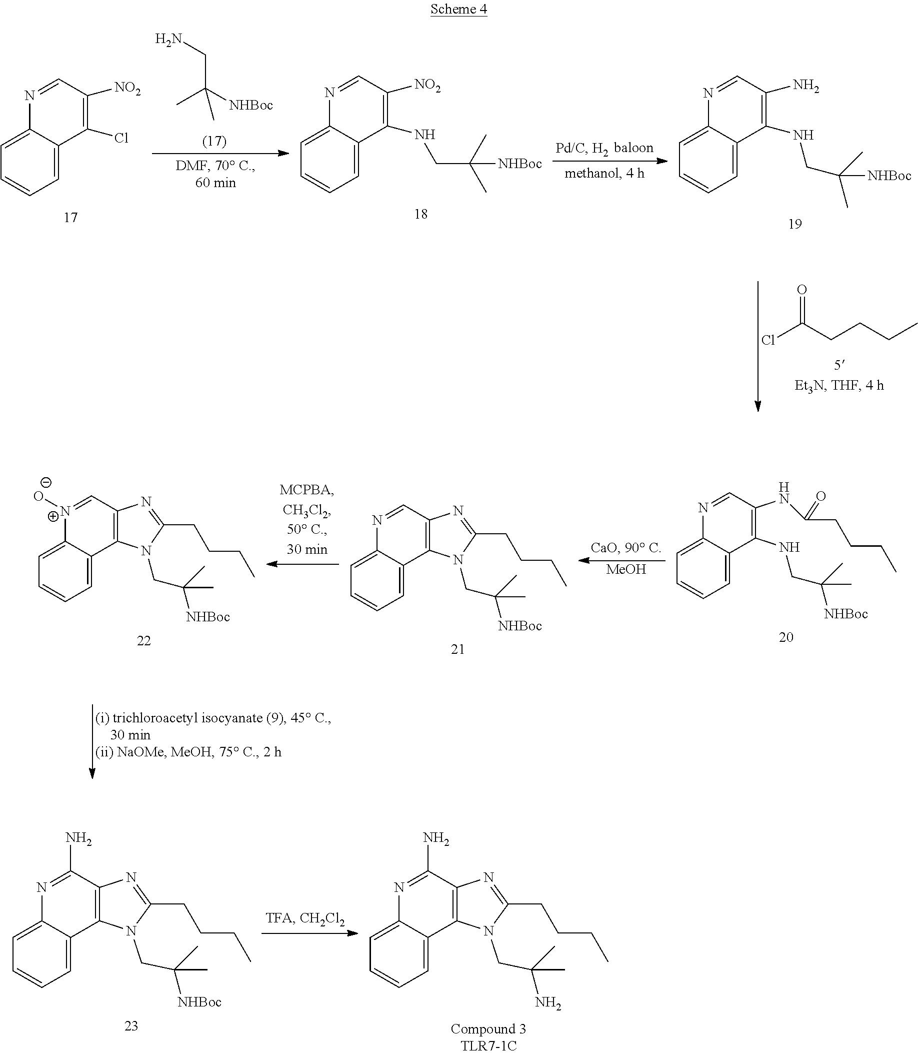

Synthetic procedure to prepare tert-butyl (3-(4-amino-2-butyl-1H-imidazo[4,5-c]quinolin-1-yl)-2,2-dimethylpropyl)carbamate (Compound 23 of Scheme 4) follows the above discussed procedure using tert-butyl (1-amino-2-methylpropan-2-yl)carbamate (Compound 17) as the starting material.

### Boc-Deprotection of tert-butyl (3-(4-amino-2-butyl-1H-imidazo[4,5-c]quinolin-1-yl)-2,2-dimethylpropyl)carbamate

Trifluoroacetic acid (10 equiv) was added to a stirred solution of tert-butyl (3-(4-amino-2-butyl-1H-imidazo[4,5-c]quinolin-1-yl)-2,2-dimethylpropyl)carbamate (100 mg, 1 equiv) (Compound 23 in Scheme 4) in 1,2-dichloromethane (1 mL). This was stirred under nitrogen atmosphere for 1 h. Once the starting materials were completely consumed as determined by TLC, it was evaporated to dryness under vacuum and basified with saturated sodium bicarbonate. This was then purified using column chromatography (MeOH/dichloromethane) to obtain 1-(2-amino-2-methylpropyl)-2-butyl-1H-imidazo[4,5-c]quinolin-4-amine (Compound 3—TLR7-1C) as a colorless liquid. LCMS: [M+H]+ m/z=312.

### Example 4: Synthesis of Folate-NHS Ester (Compound 25)

Synthesis of a releasable TLR7-folate conjugate is described in Scheme 5:

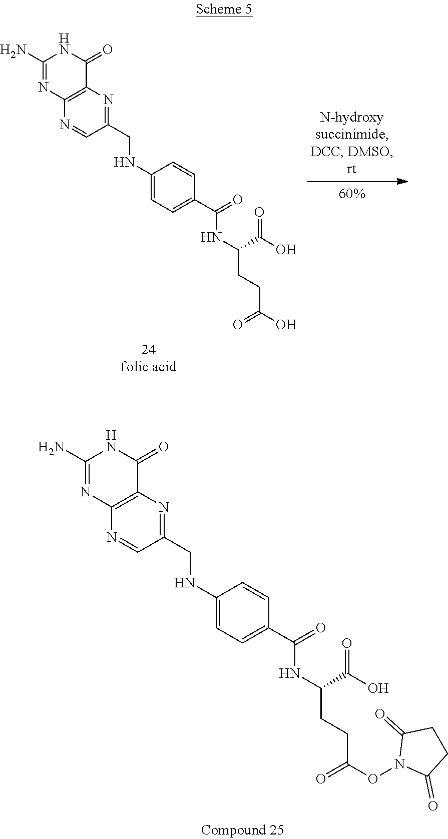 [SMILES: Nc1nc2ncc(CNc3ccc(C(=O)N[C@@H](CCC(=O)O)C(=O)O)cc3)nc2c(=O)[nH]1 ; Nc1nc2ncc(CNc3ccc(C(=O)N[C@@H](CCC(=O)ON4C(=O)CCC4=O)C(=O)O)cc3)nc2c(=O)[nH]1]

DMSO (20 mL), dicyclohexylcarbodiimide (0.94 g), triethylamine (0.5 mL) and N-hydroxysuccinimide (0.52 g) was added to a stirred solution of folic acid (1 g) (Compound 24 of Scheme 5). This was stirred under nitrogen atmosphere for about 12 hours. The solution was filtered to remove dicyclohexyl urea biproduct. Folate-NHS (Compound 25) was precipitated with the excess of ethyl acetate and washed three times with anhydrous ether, dried under vacuum and stored at −20 C. Yield was 60%.

### Example 5: Synthesis of Non-Releasable TLR7-Folate Conjugate-1 (Compound 4)

Synthesis of non-releasable TLR7-folate conjugates (e.g., Compound 4) is described in Scheme 6:

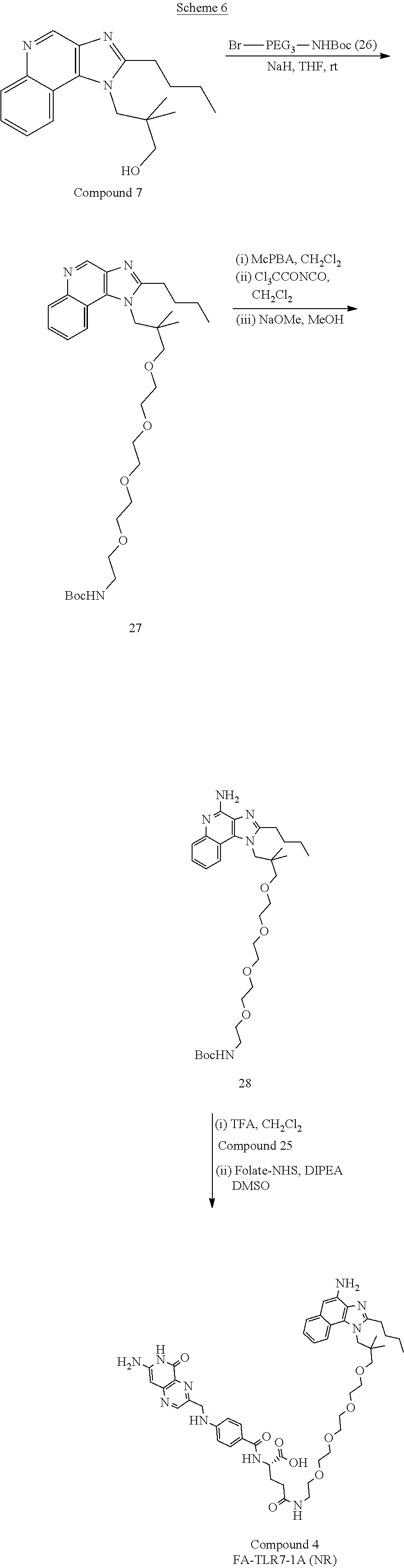

### (S)-1-(4-amino-2-butyl-1H-imidazo[4,5-c]quinolin-1-yl)-20-(4-(((2-amino-4-oxo-3,4-dihydropteridin-6-yl)methyl)amino)benzamido)-2,2-dimethyl-17-oxo-4,7,10,13-tetraoxa-16-azahenicosan-21-oic acid (Compound 4; FA-PEG₃-TLR7-1A Non-Releasable (“NR”))

### Step 1: tert-butyl (15-(2-butyl-1H-imidazo[4,5-c]quinolin-1-yl)-14,14-dimethyl-3,6,9,12-tetraoxapentadecyl)carbamate (Compound 27 in Scheme 6)

NaH (2 equiv) and N-Boc-PEG₃-bromide (2 equiv) (Compound 26 in Scheme 6) were added to a stirred solution of the intermediate hydroxyl compound 7 (500 mg) synthesized above (Scheme 2) in THF (5 mL). This was stirred under nitrogen atmosphere for about 5 hours and thereafter the solvent was evaporated to dryness using rotary evaporator. It was then quenched with water and diluted with dimethyl sulfoxide (DMSO). The crude reaction mixture was purified through HPLC using ammonium acetate and acetonitrile as the mobile phase to get the Compound 27 (see Scheme 6) as a colorless liquid. Yield was 60%.

### Step 2: tert-butyl (15-(4-amino-2-butyl-1H-imidazo[4,5-c]quinolin-1-yl)-14,14-dimethyl-3,6,9,12-tetraoxapentadecyl)carbamate (Compound 28)

3-chloroperoxybenzoic acid (1.5 equiv) was added to a stirred solution of Compound 27 (100 mg) in anhydrous dichloromethane (1 mL), and the solution was refluxed at 45° C. for 30 minutes. Once the starting material was completely consumed, the solvent was evaporated to dryness under vacuum. The residue was then re-dissolved in anhydrous dichloromethane (1 mL), followed by the addition of trichloroacetyl isocyanate (2.0 equiv) and the reaction mixture was heated at 45° C. for 30 minutes.

After the completion of the reaction, the solvent was removed under vacuum and the residue was re-dissolved in anhydrous MeOH (1 mL), followed by the addition of 25% methanolic sodium methoxide solution (0.2 mL). This was then heated at 75° C. for an hour and cooled down. The solvent was removed under vacuum and the residue was purified using column chromatography (MeOH/dichloromethane) to obtain the compound 28 as colorless liquid. Overall yield was 40%.

### Step 3: 1-(1-amino-14,14-dimethyl-3,6,9,12-tetraoxapentadecan-15-yl)-2-butyl-1H-imidazo[4,5-c]quinolin-4-amine

Trifluoroacetic acid (10 equiv) was added to a stirred solution of the intermediate Compound 28 (100 mg) in dichloromethane (1 mL). This was stirred under nitrogen atmosphere for about 1 hour and the solvents were evaporated to dryness using a rotary evaporator. After the complete removal of residual acid, the residue was dissolved in DMSO (1 mL) to give a DMSO solution.

### Step 4: (S)-1-(4-amino-2-butyl-1H-imidazo[4,5-c]quinolin-1-yl)-20-(4-(((2-amino-4-oxo-3,4-dihydropteridin-6-yl)methyl)amino)benzamido)-2,2-dimethyl-17-oxo-4,7,10,13-tetraoxa-16-azahenicosan-21-oic acid (Compound 4)

To the DMSO solution from the prior step, folate-NHS (2.0 equiv) (Compound 25 from Scheme 5) and N,N-diisopropylethylamine (2 equiv) were added and stirred for 60 minutes. The reaction was monitored through LCMS. After the starting material was completely consumed, the product was purified through HPLC using ammonium acetate and acetonitrile as the mobile phase to get the product Compound 4 as a yellow solid. Yield was 70%. The product was confirmed using LCMS. Calculated mass for C46H60N12O9 is 924.46, obtained mass [M+H]+ is 925.

### Example 6: Synthesis of Folate-PEG₃-Cysteine Conjugates (Compound 33)

Synthesis of folate-PEG₃-cysteine conjugates (e.g., Compound 33) is described in Scheme 7:

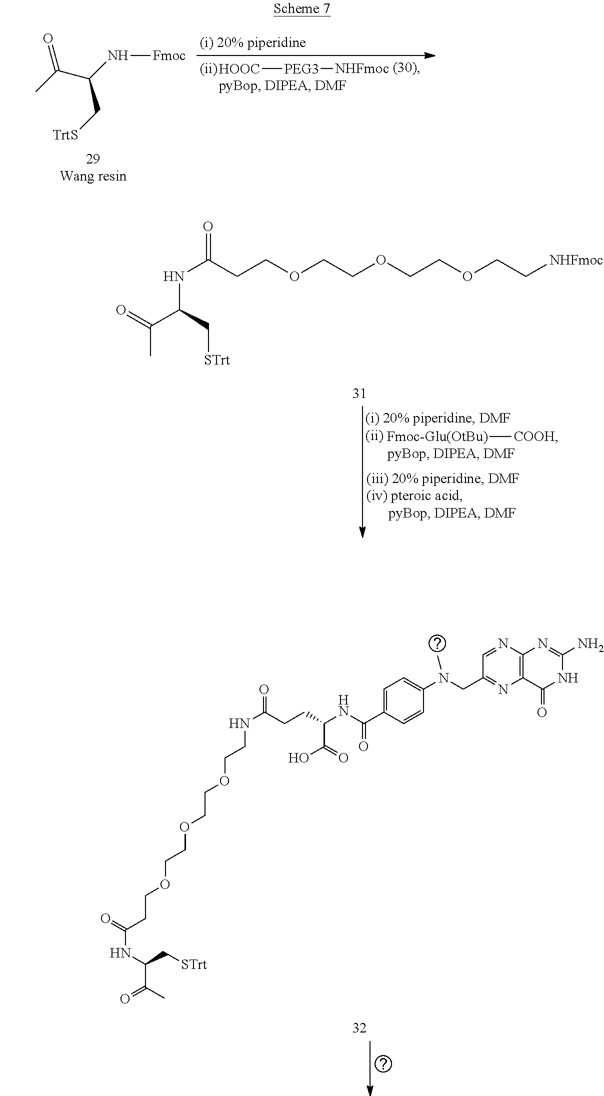

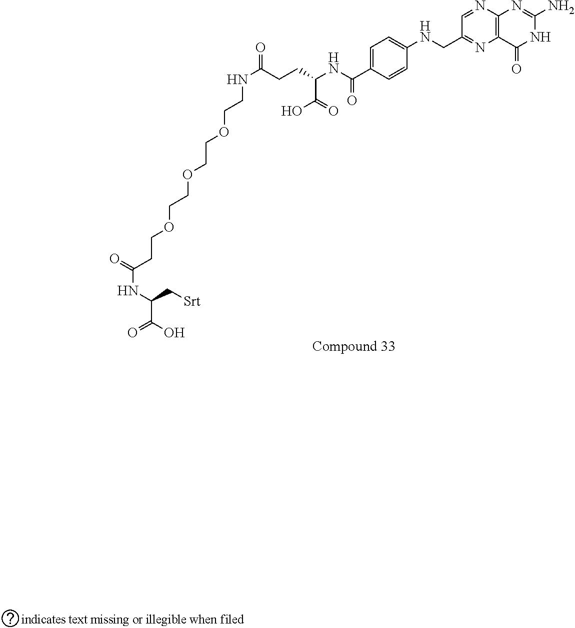

NH-Fmoc-Cys(Trt)-Wang resin (Compound 29) was initially deprotected using 20% piperidine in DMF (3×10 minutes). The free amine was treated with F_{moc}-PEG₃-acid (1.5 equiv) (Compound 30) in the presence of benzotriazole-1-yloxytripyrrolidinophosphonium hexafluorophosphate (PyBop) (2 equiv), diisopropylethylamine (DIPEA) (3 equiv) and dimethylformamide (DMF). Once the starting material was consumed, resin was washed thrice with DMF, to give Compound 31.

This was then deprotected using 20% piperidine in DMF (3×10 minutes) and washed with DMF. The free amine obtained was treated with Fmoc-Glu(O^{t}Bu)-COOH (2 equiv) in presence of PyBop (2 equiv), DIPEA (3 equiv) and DMF. The coupled product was deprotected using 20% piperidine in DMF (3×10 minutes) and treated with pteroic acid (1.5 equiv) in presence of PyBop (2 equiv), DIPEA (2 equiv) and DMF. Once the coupling was complete to give Compound 32, the trifluoroacetyl group was deprotected with 50% ammonia-DMF solution (3×20 minutes). Finally, the resin was cleaved using a TFA:TIPS:water:TCEP cocktail solution and purified using HPLC to get the folate-PEG₃-cysteine (Compound 33) as a yellow color solid.

For HPLC purification, 20 mM ammonium acetate at pH-5 and acetonitrile was used as mobile phase.

### Example 7: Synthesis of Compounds 35 and 36

Synthesis of Compounds 35 and 36 is described in Scheme 8:

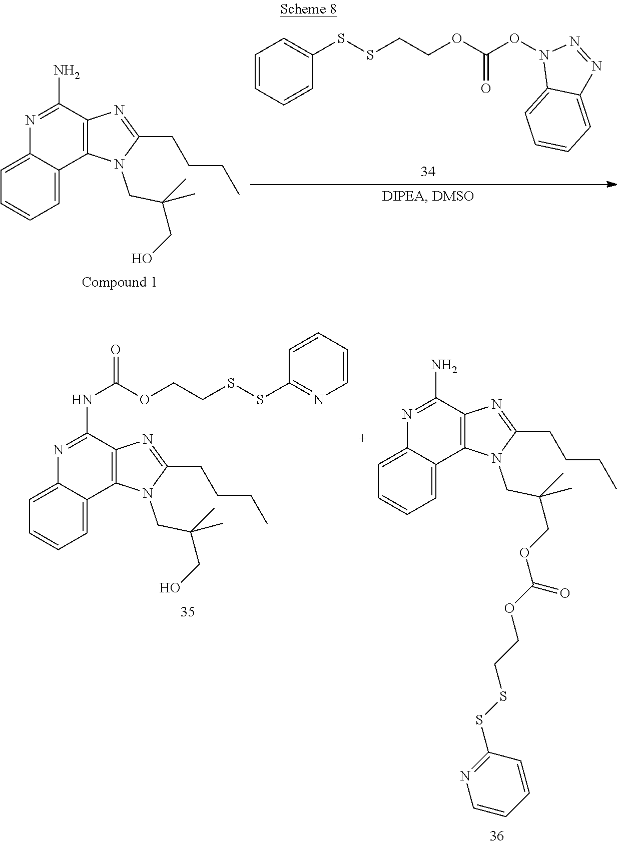

### Synthesis of 3-(4-amino-2-butyl-1H-imidazo[4,5-c]quinolin-1-yl)-2,2-dimethylpropyl (2-(pyridin-2-yldisulfaneyl)ethyl) carbonate (Compound 36 in Scheme 8)

Heterobifunctional linker (2 equiv) (Compound 34) was added to a solution of TLR7-1 (Compound 1; 1 equiv) and 4-dimethylaminopyridine (DMAP) (0.2 equiv) in 1 mL of methylene dichloride at room temperature under N₂ atmosphere. It was then stirred at room temperature for 12 hours and purified with HPLC using ammonium acetate, methanol mobile phase to obtain Compounds 35 and 36 in Scheme 8.

### Example 8: Synthesis of Releasable TLR7-Folate Conjugates

Synthesis of certain releasable TLR7-folate conjugates (e.g., Compound 5) is described in Scheme 9:

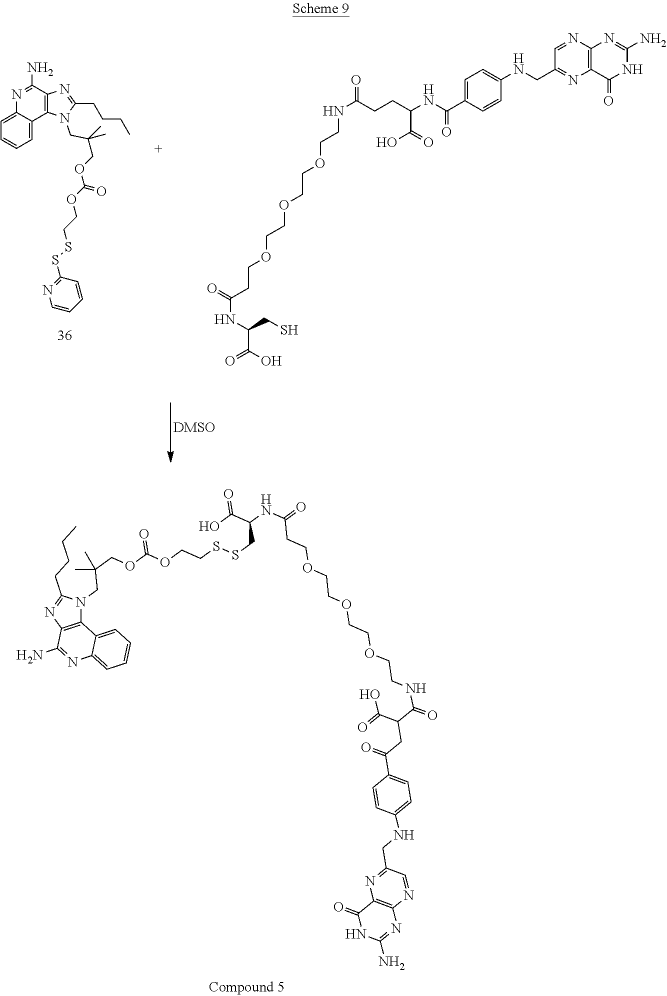 [SMILES: CCCCc1nc2c(N)nc3ccccc3c2n1CC(C)(C)COC(=O)OCCSSc1ccccn1 ; Nc1nc2ncc(CNc3ccc(C(=O)NC(CCC(=O)NCCOCCOCCOCCC(=O)N[C@@H](CS)C(=O)O)C(=O)O)cc3)nc2c(=O)[nH]1 ; CCCCc1nc2c(N)nc3ccccc3c2n1CC(C)(C)COC(=O)OCCSSC[C@H](NC(=O)CCOCCOCCOCCNC(=O)C(CC(=O)c1ccc(NCc2cnc3nc(N)[nH]c(=O)c3n2)cc1)C(=O)O)C(=O)O]

Folate-PEG₃-Cysteine (Compound 33, 1.2 equiv) was added to a stirred solution of compound 36 (1 equiv) in DMSO and stirred under nitrogen atmosphere for about 1-2 hours. LCMS analysis of the mixture indicated the formation of Compound 5 (FA-PEG₃-TLR7-1A (Re)). The mixture was then purified by preparative HPLC using ammonium acetate and acetonitrile as mobile phase.

### Example 9: Synthesis of non-releasable TLR7-Folate Conjugate

Synthesis of certain non-releasable TLR7-folate conjugates (e.g., Compound 9) is described in Scheme 10:

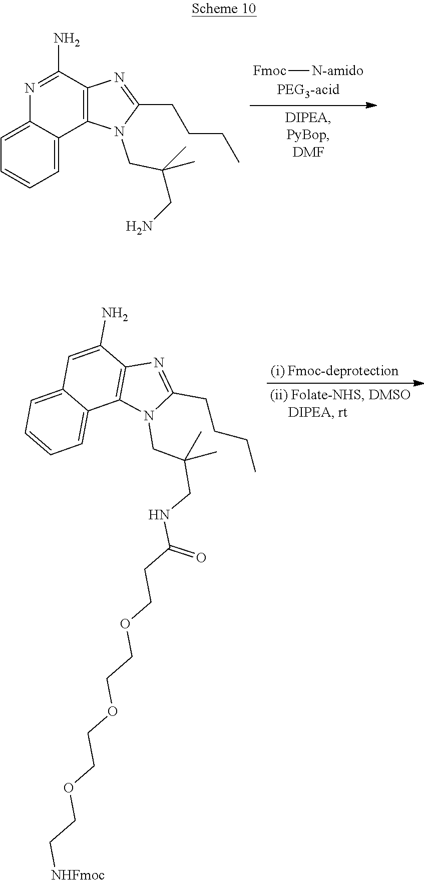

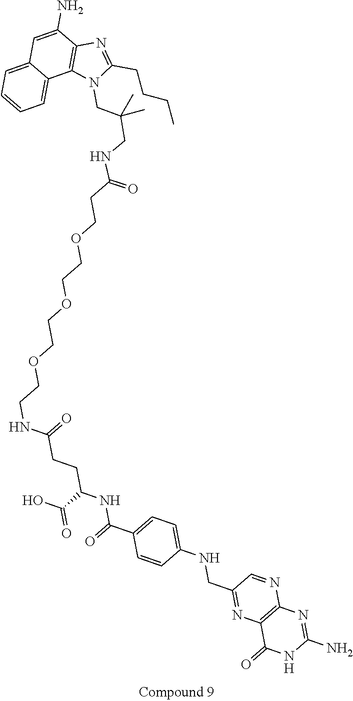

### Synthesis of (9H-fluoren-9-yl)methyl (16-(4-amino-2-butyl-1H-imidazo[4,5-c]quinolin-1-yl)-15,15-dimethyl-12-oxo-3,6,9-trioxa-13-azahexadecyl)carbamate

Fmoc-N-amido PEG₃ acid (1.5 equiv), PyBop (2 equiv) and DIPEA (2 equiv) were added to a stirred solution of 1-(3-amino-2,2-dimethylpropyl)-2-butyl-1H-imidazo[4,5-c]quinolin-4-amine (Compound 2; 1 equiv) in DMF. This was stirred under nitrogen atmosphere for about 2 hours and purified using HPLC to get the title compound.

### Step 2: Synthesis of (S)-1-(4-amino-2-butyl-1H-imidazo[4,5-c]quinolin-1-yl)-21-(4-(((2-amino-4-oxo-3,4-dihydropteridin-6-yl)methyl)amino)benzamido)-2,2-dimethyl-5,18-dioxo-8,11,14-trioxa-4,17-diazadocosan-22-oic acid (compound 9)

Tris (2-aminoethyl) amine (10 equiv) was added to a stirred solution of (9H-fluoren-9-yl)methyl (16-(4-amino-2-butyl-1H-imidazo[4,5-c]quinolin-1-yl)-15,15-dimethyl-12-oxo-3,6,9-trioxa-13-azahexadecyl)carbamate (1 equiv) in THF. This was stirred for about 1 hour and the reaction monitored by LCMS. Once the starting materials were completely consumed, it was purified using HPLC. The purified sample (1 equiv) was dissolved in DMSO and treated with folate-NHS (Compound 25 in Scheme 5, 2 equiv) and DIPEA (2 equiv) for 2 hours. This was then purified using HPLC to get the non-releasable TLR7-Folate conjugates (Compound 9).

### Example 10: Efficacy in Human Cells

To evaluate the efficacy of TLR-7 agonists, Compounds 1 (TLR7-1A), 2 (TLR7-1B) and 3 (TLR7-1C) were treated with human peripheral blood mononuclear cells (PBMCs) for 24 hours and cytokine production was thereafter assessed. Human PBMCs were isolated from healthy donors by density gradient centrifugation following standard procedure. 18 mL of blood were diluted with 2% fetal bovine serum (FBS) in phosphate-buffered saline (PBS) (1:1 dilution) and transferred slowly into a 50 mL SepMate™ PBMC isolation tube containing 12 mL of Ficoll-Paque®. The tube was centrifuged at 1200 g for 10 minutes and PBMCs were transferred into a fresh 50 mL tube.

PBMCs were washed with 2×50 mL of 2% FBS in PBS and plated in 96 well plate at a density of 2×10⁵ cells/200 μL RPMI medium. This was treated with different concentrations of TLR7 agonist for 24 hours.

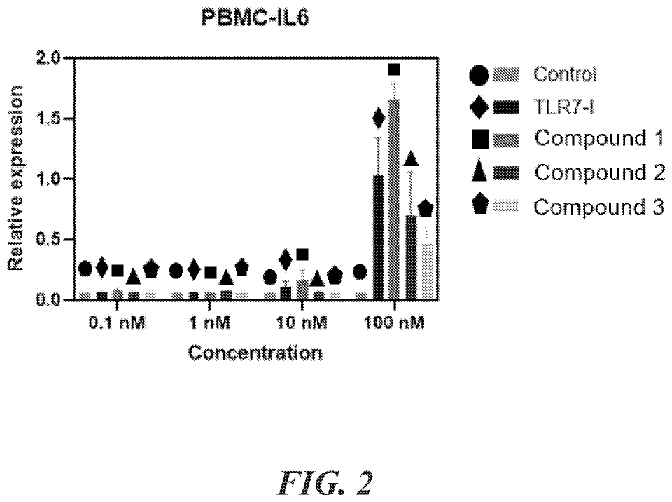

Compound A (TLR7-1) was used as control. Cell culture supernatants were isolated and cytokine levels were calculated using an enzyme-linked immunosorbent assay (ELISA) kit (FIG. 2). As shown in FIG. 2, the TLR7 agonists (Compounds 1, 2, and 3) resulted in increased expression of IL-6 in PBMCs as compared to the control.

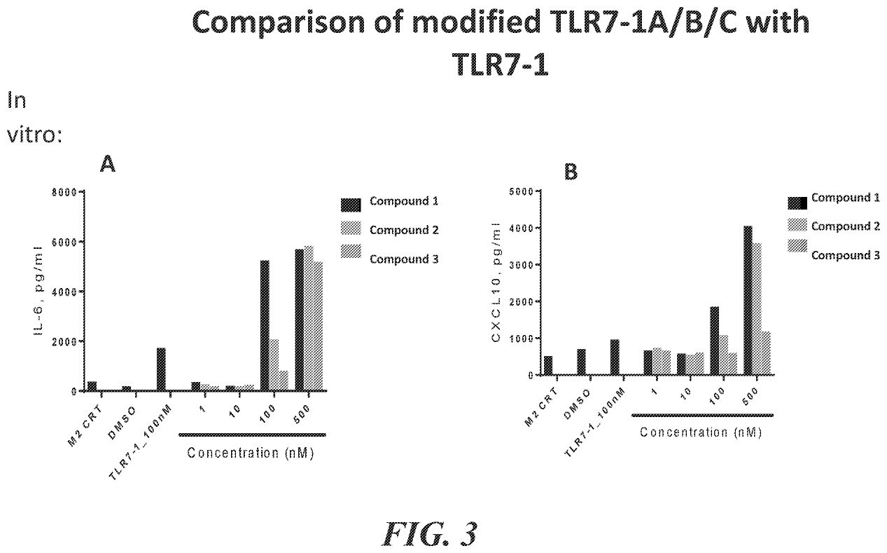

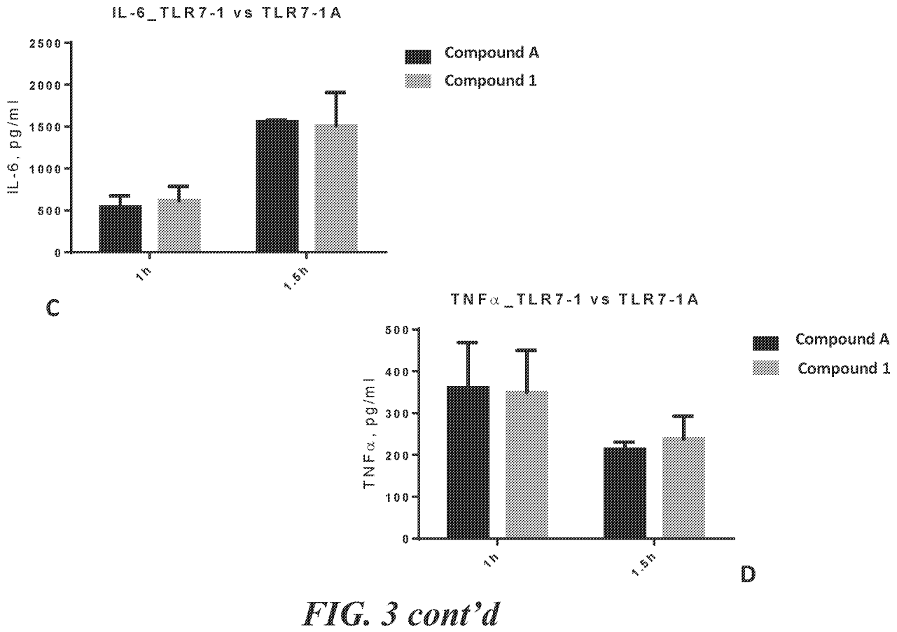

When Compounds 1, 2, and 3 were treated with human primary monocyte derived M2-macrophages for 48 hours, they induced interleukin-6 (IL-6) and C-X-C motif chemokine ligand 10 (CXCL-10) more efficiently compared to the parent compound TLR7-1 (FIGS. 3A and 3B). Compounds 1, 2, and 3 polarize the M2 macrophages to M1 macrophages as shown by the increased M1 markers IL-6 (FIG. 3A) and CXCL10 (FIG. 3B).

### Example 11: Efficacy in Mice

Female Balb/c mice were purchased from Charles Rivers, housed in a sterile environment on a standard 12-hour light-dark cycle, and maintained on a folate-deficient diet. All animal procedures were approved by the Purdue Animal Care and Use Committee in accordance with National Institutes of Health guidelines.

Healthy mice were tail vein injected with 10 nmol Compound A (TLR7-1) or Compound 1 (TLR7-1A), and peripheral blood was collected at indicated time points after drug injection. (FIGS. 3C and 3D). The effect of drug on plasma levels of IL-6 (FIG. 3C), and tumor necrosis factor alpha (TNF-α) (FIG. 3D) was determined at 1 hour or 1.5 hours after treatment. Both Compounds A and 1 stimulated systemic cytokine release in healthy mice.

### Example 12: Macrophage Polarization

Bone marrow cells were isolated from tibias and femurs of male C57BL/6 mice and were differentiated to macrophages with mouse M-CSF (20 ng/mL). The macrophages were then polarized to M2 phenotype with 20 ng/mL of IL-4/IL-6/IL-13 for 48 hours. Interferon-γ (IFN-γ), interleukin-4 (IL-4), and interleukin-13 (IL-13) were obtained from Biolgend (San Diego, Calif.). Lipopolysaccharide (LPS) was purchased from Sigma-Aldrich (St. Louis, Mo.).

The resulting M2 macrophages were then incubated with varying concentrations of Resiquimod, GSK2245035, Compound 1, Compound 2, and Compound 3 for 48 hours. Supernatant was harvested and cytokines were analyzed using ELISA. (Resiquimod and GSK2245035 are known TLR7 agonists.)

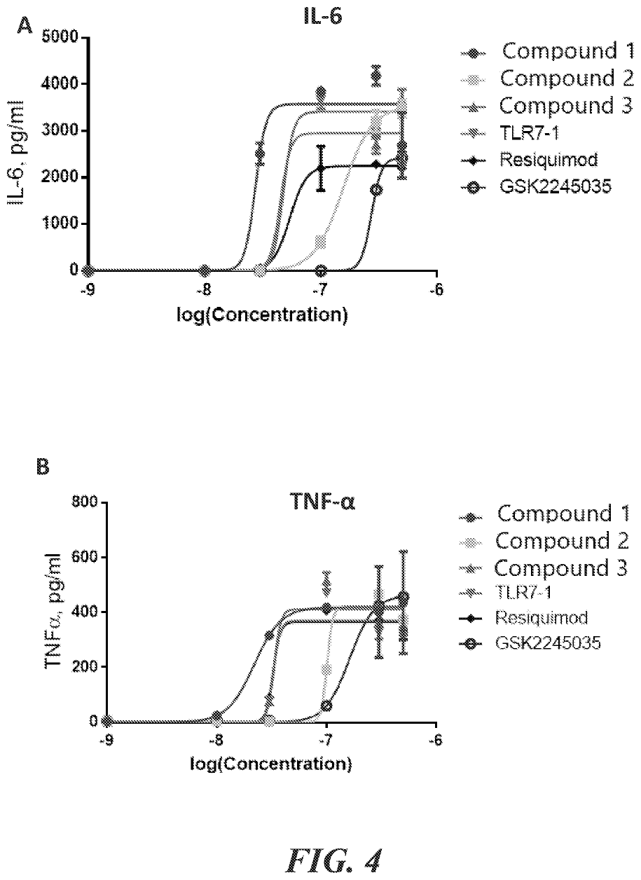

As shown in FIGS. 4A and 4B and the increase in M1 markers IL-6 and TNF-α, Compound 1 efficiently polarized the macrophages from the M2 to M1 phenotype.

### Example 13: Expression of FR-β and TLR7 in Human M2 Macrophages

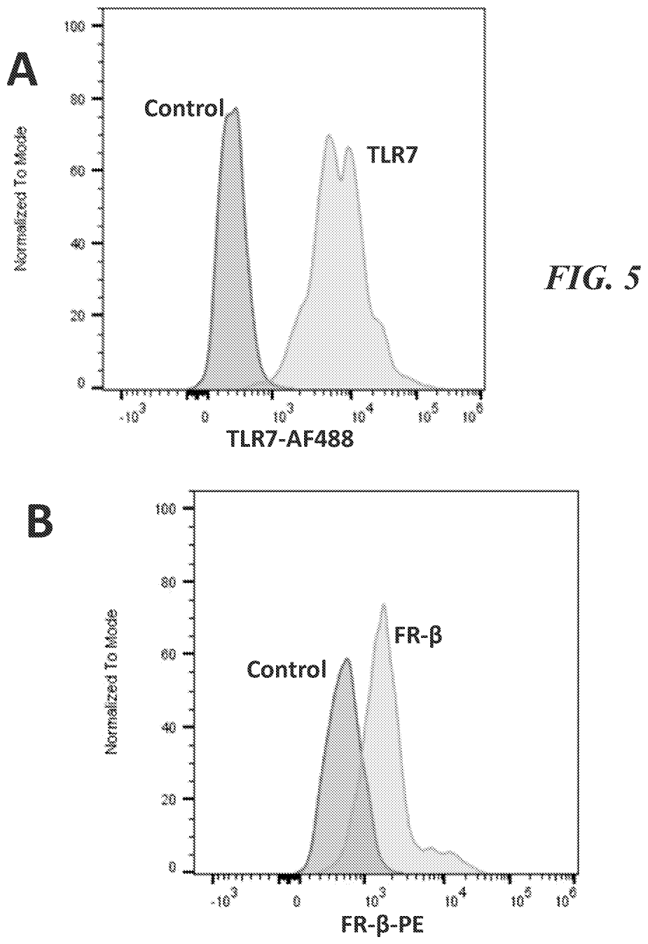

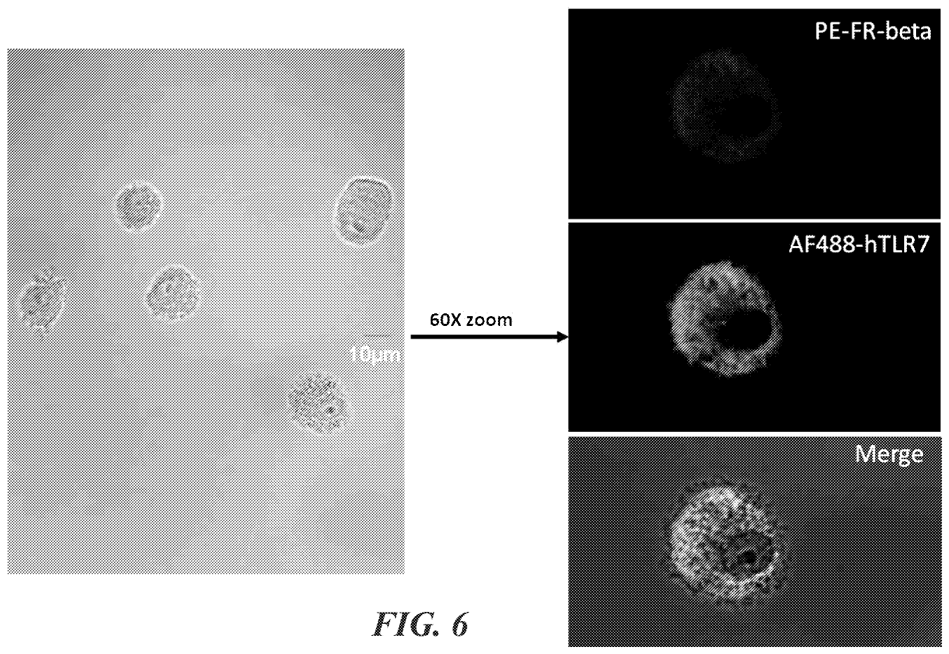

M2-polarized macrophages obtained above were detached using Accutase cell detachment solution (Biolegend, San Diego, Calif.) and gently lifted with a cell scraper. Cells were then washed with PBS and nonspecific binding was blocked by incubation with Fc receptor blocking solution (Biolegend, San Diego, Calif.) for 10 minutes. Cells were then washed with PBS and resuspended in 150 μl of Cyto-Fast™ Fix/Perm Buffer (Biolegend, San Diego, Calif.). This was incubated for 20 minutes at room temperature and washed with 1 mL of 1× Cyto-Fast perm wash solution. It was centrifuged at 350 g for 5 minutes and the supernatant was discarded. Cocktail of TLR7 and folate receptor beta (FR-β) antibodies was prepared in 1× Cyto-Fast™ Perm Wash Solution and 100 mL of this was incubated with the cell suspension for 20 minutes in the dark. Cells were washed with 1 ml of perm wash solution and resuspended in staining buffer. This was used for flow cytometry and confocal microscopic images. FIGS. 5A and 5B confirm that the M2 macrophages express TLR7 and FR-β, and FIG. 6 shows that both TLR7 and FR-β co-localize into the same endosome of macrophages.

### Example 14: Disulfide Cleavage Study

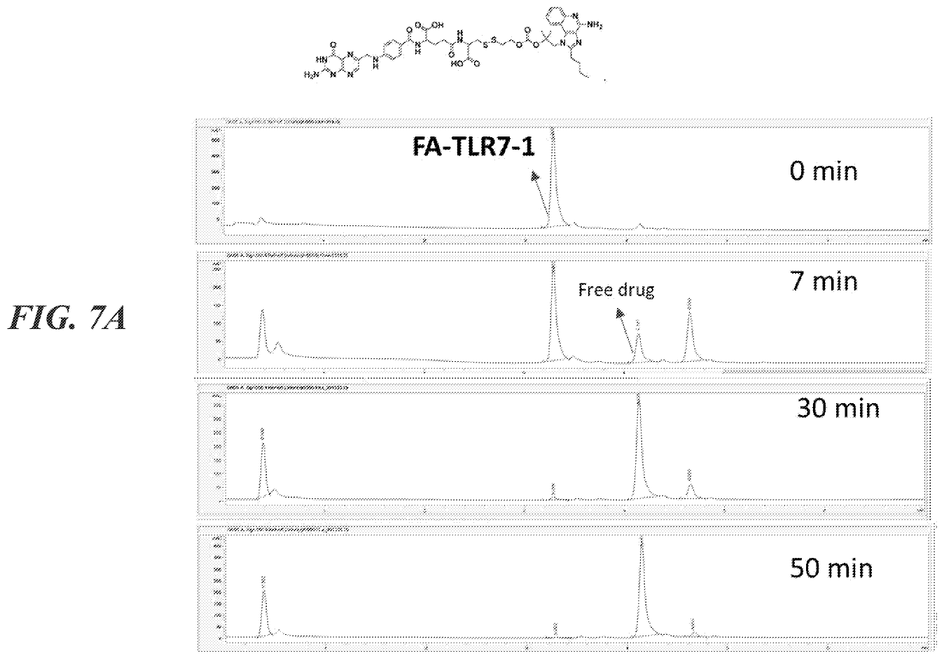

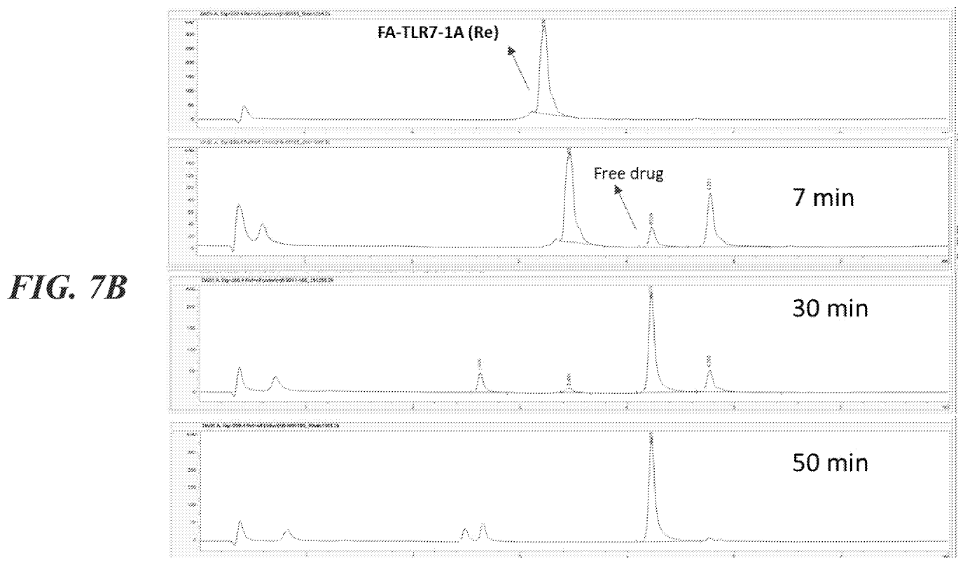

The stability kinetics of folate-TLR7 conjugates were assessed, with FIG. 7A showing FA-Compound A (FA-TLR7-1) and FIG. 7B showing Compound 5 (releasable conjugate (“Re”)) in the presence of thiol. Synthesis of FA-TLR7-1 is described in WO2021007277A1, which publication is incorporated by reference herein in its entirety. Both conjugates were treated with dithiothreitol (DTT) (40 equiv) in PBS. Samples were withdrawn and analyzed by LCMS at 0 minutes, 7 minutes, 30 minutes and 50 minutes. As shown in FIGS. 7A and 7B, both conjugates rapidly cleaved in the presence of DTT and released the free-drug (e.g., the immune modulator; Compound A) within 30 minutes.

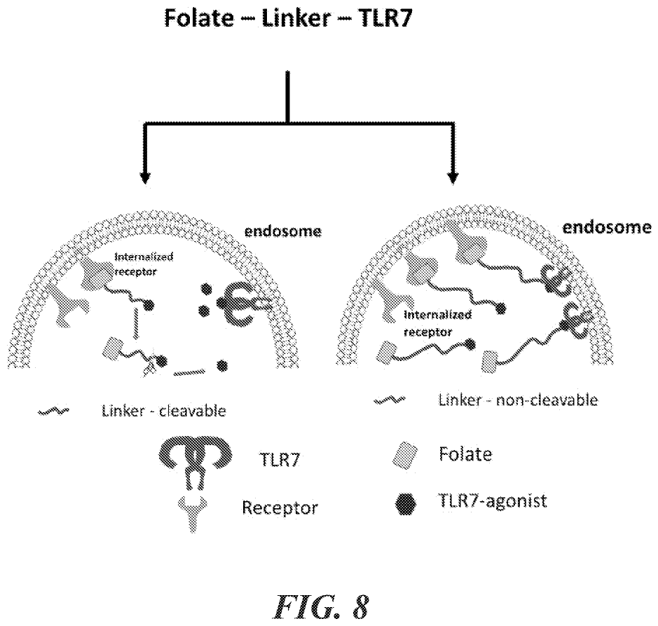

FIG. 8 shows a schematic diagram of the mechanism of action of releasable and non-releasable folate-TLR7 conjugates. A releasable conjugate can release the free drug TLR7 agonist upon disulfide cleavage in the endosome. Since TLR7 and FR-β present in the same endosome, a non-releasable/non-cleavable conjugate can induce the immune response without cleavage. This can avoid premature drug release, promote stability of the compound in circulation, increase its endosomal residence time, and ultimately require less dosing to achieve a therapeutic effect.

### Example 15: Efficacy Studies

To evaluate the efficacy of the releasable and non-releasable conjugates, freshly isolated PBMCs were plated with monocyte attachment medium at a seeding density of 1 million cells/cm². This was incubated for about 2 hours at 5% CO₂ and 37° C. in the incubator. Non-adherent cells were removed, and the monocytes were washed three times with warm monocyte attachment medium. The monocytes were cultured in folate deficient RPMI 1640 medium. This was differentiated into unpolarized macrophages by incubating with fresh RPMI medium containing 20 ng/mL of M-CSF supplemented with 1% penicillin streptomycin (Invitrogen, Carlsbad, Calif.) and 10% FBS.

After 3 days, the medium was replaced with fresh RPMI medium containing 20 ng/mL of M-CSF. On day 7, the resulting macrophages were then polarized to M2 macrophages by incubating with 20 ng/ml IL-4 and 20 ng/ml IL-13 for 2 days.

Macrophages were detached using Accutase™ cell detachment solution and seeded into 96-well plates at a density of 60,000 cells/well. To evaluate whether a TLR7 agonist or its conjugate can reprogram the M2-like macrophages into a M1-phenotype, cells were incubated with different concentrations of TLR7 agonist (Compound A; TLR7-1) or its corresponding conjugates (e.g., FA-PEG₃-TLR7-1A conjugates, releasable (Compound 5) and non-releasable (Compound 4)).

Different concentrations of either TLR7 agonist or its folate conjugates (e.g., Compounds 4 and 5) were incubated with the above polarized M2-like macrophages for the indicated times. The culture medium was harvested for analysis of secreted cytokines and the collection of cells for qPCR analysis. Total RNA was isolated from around 2×10⁵ macrophages using Quick-RNA™ MicroPerp kit (Zymo Research, Irvine, Calif.) according to the manufacturer-recommended protocol. The RNA samples were then reverse-transcribed into cDNA using high-capacity cDNA reverse transcription kits (Applied Biosystems, Foster City, Calif.; #4368814).

qPCR analyses were performed using the iTaq™ Universal SYBR Green SuperMix (Bio-Rad Laboratories, Inc., Hercules, Calif.; #1725121), iCycler thermocycler, and iCycler iQ 3.0 software (Bio-Rad Laboratories Inc., Hercules, Calif.) to track the expression of markers characteristic of macrophage polarization states. IL-6 and TNF-α were used as markers for the M1 phenotype. Each sample was analyzed independently in triplicate for each marker. IL-6, CXCL-10 and TNF-α protein expression in the cell culture supernatants were calculated using the commercially available ELISA kits (Biolegend, San Diego, Calif.).

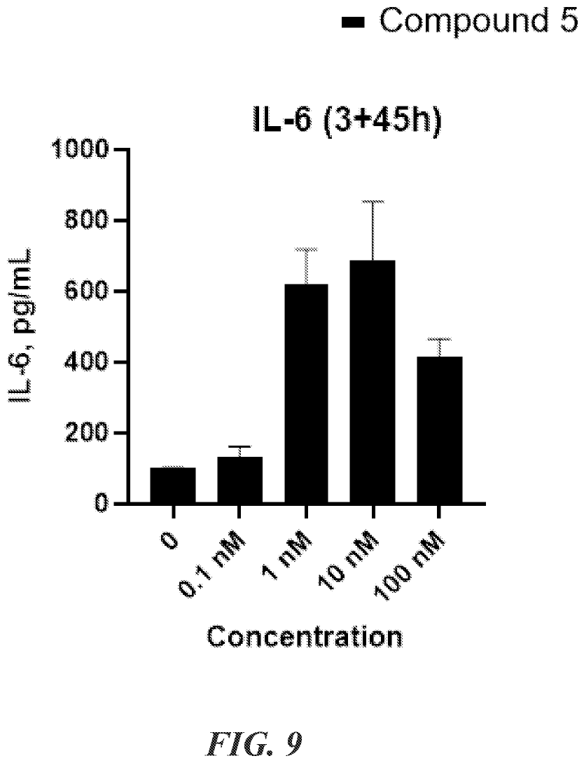

FIG. 9 shows the results of Compound 5 treated with human PMBC-derived M2 macrophages for 3+45 hours, evidencing that treatment with Compound 5 polarized the M2 macrophages to M1 macrophages (evidenced by the increased M1 markers).

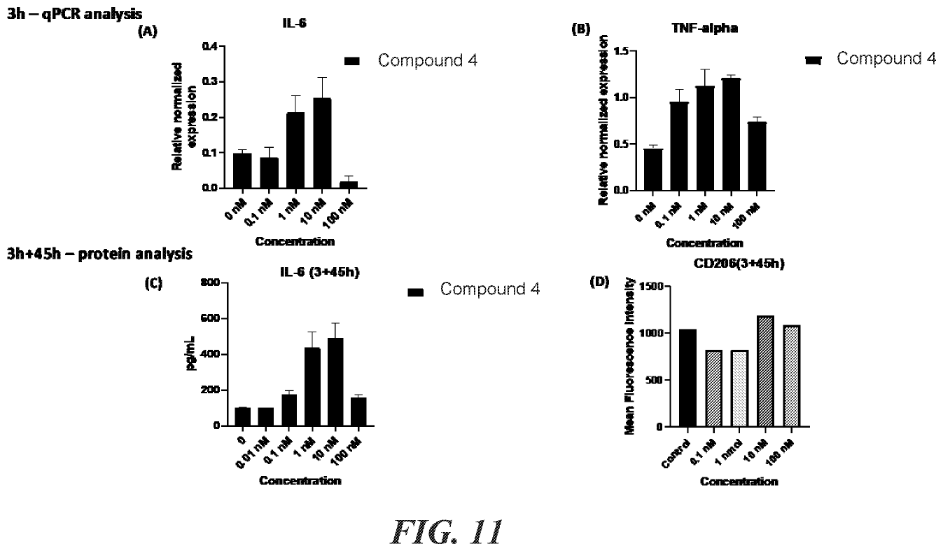

FIGS. 11A-11D show the results of Compound 4 treated with human PMBC-derived M2 macrophages for either 3 hours or 3+45 hours. In the latter case, after 3 hours of incubation, the cell culture medium was replaced with drug-free fresh medium and incubated for an additional 45 hours. As supported by FIGS. 11A-11D, Compound 4 polarized the M2 macrophages to M1 macrophages (evidenced by the increased M1 markers).

### Example 16: Analysis of Surface Markers During Macrophage Polarization

Human PMBC-derived M2 macrophages were treated with different concentrations of folate-TLR7 agonists (e.g., Compound 4) for 48 hours, and macrophage cells were detached using Accutase cell detachment Solution (Biolegend, San Diego, Calif.; #423201) and gently lifted with a cell scraper. Cells were washed with PBS and nonspecific binding was blocked by incubation with Fc receptor blocking solution (Biolegend, San Diego, Calif.) at room temperature for 10 minutes. The resulting single cell suspensions were stained for M2 macrophage marker CD206, and M1 macrophage markers CD80 and CD40. All the samples were then analyzed by flow cytometry.

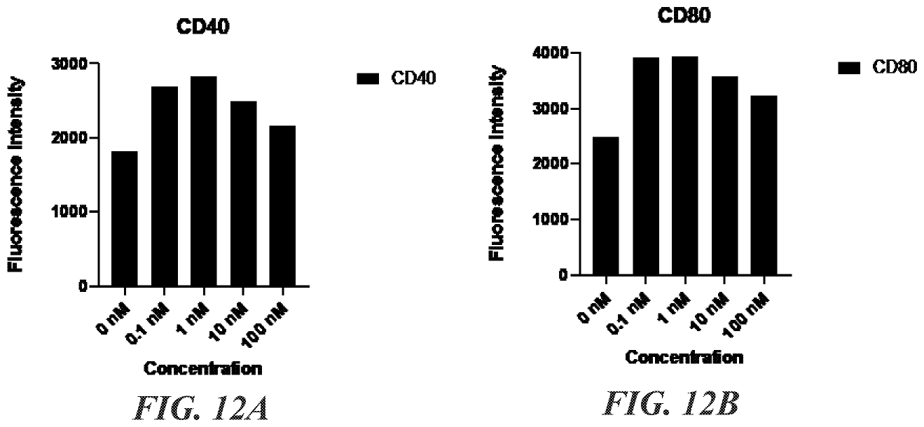

As shown in FIGS. 12A and 12B, the non-releasable conjugate Compound 4 polarized the M2 macrophages to M1 macrophages as shown by the increased M1 surface markers CD40 and CD80.

### Example 17: In Vivo Efficacy Studies

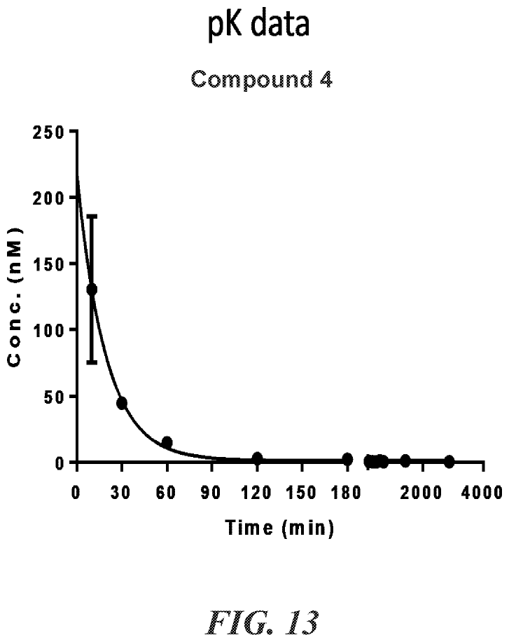

Referring to FIG. 13, 20 nmol/mouse of a Folate-TLR7-1A conjugate (Compound 4; FA-PEG₃-TLR7-1A (NR)) was injected into healthy C57BL/6-NCrl mice intravenously. Blood was collected at 10, 30, 60, 120, 180, 250, 360, 480, 600, 720, 1440, and 2880 minutes post drug injection. Blood was centrifuged at 1000 g for 10 minutes and plasma was collected carefully into a clean tube. Acetonitrile was added to the plasma (acetonitrile/plasma sample=2/1 (v/v)), followed with internal standard (0.5 ng resiquimod). The mixture was vortexed thoroughly and centrifuged at 13,000 rpm for 10 minutes. Supernatant was collected and injected to Agilent 6410 NanoLC QQQ to obtain the pharmacokinetic analysis results shown in FIG. 13.

In additional in vivo studies, 6-8 week old female BalB/c mice (Charles River Laboratories International, Inc., Wilmington, Mass.) were transferred upon arrival to a folic acid-deficient diet (TD.95247, Envigo Corporation, Indianapolis, Ind.). On day 14, mice were implanted subcutaneously with 5.0×10⁴ of 4T1 cells and tumors were allowed to grow until they reach ˜50 mm³. Mice were then injected intravenously with different concentrations of 100 μl of Folate-TLR7 conjugate Compounds 4 or 10 nmol/mouse of Compound 5). Control groups received 100 μl of 3% DMSO in PBS on the same schedule.

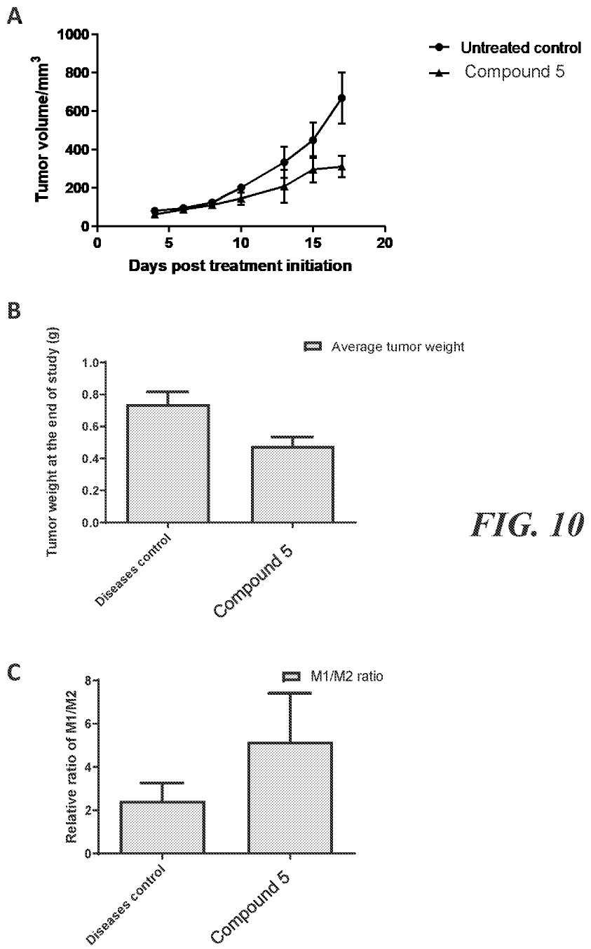

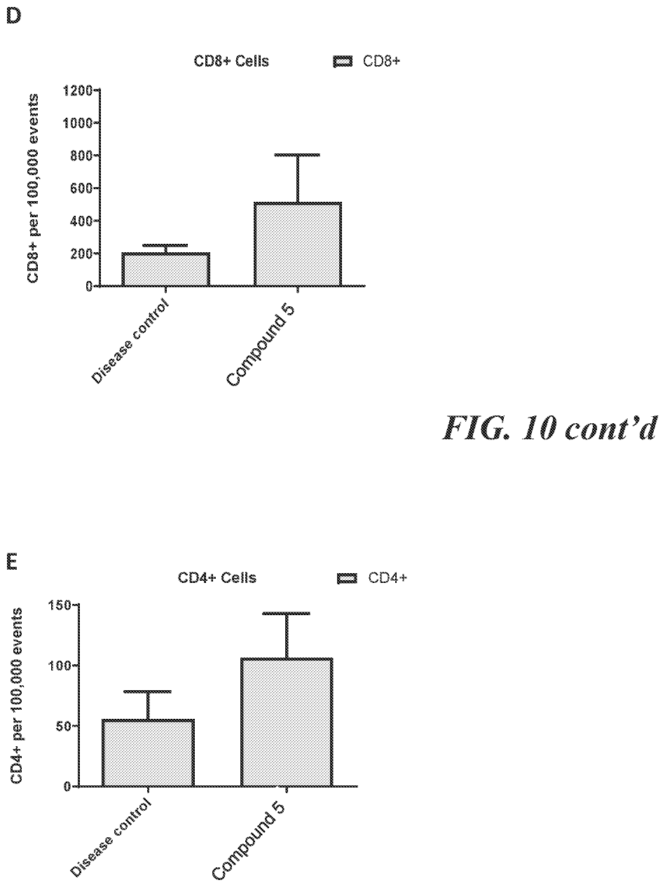

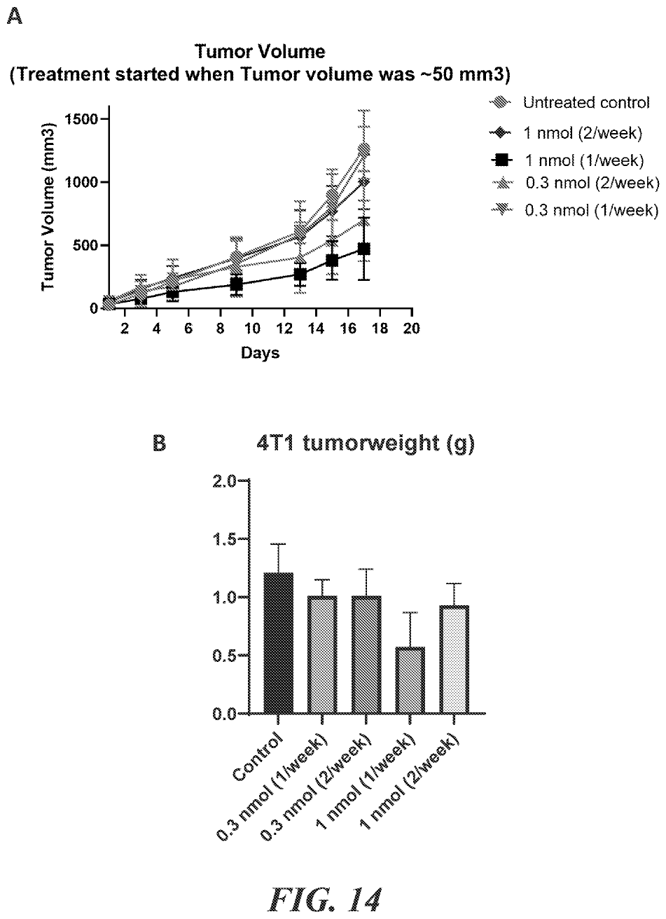

Tumor volume was concurrently measured with calipers using the formula (a×b²)/2 (a being the largest and b being the smallest diameter of the tumor) (see FIGS. 10A, 10B, and 14). When desired, mice were sacrificed, and derived tumor fragments were dissociated using a human tumor dissociation kit. The resulting single cell suspensions were stained for antibodies and the samples were then analyzed by flow cytometry. Percentage of CD4 and CD8 T cell populations were tested in live cells isolated from 4T1 solid tumors in groups of untreated control and Folate-TLR7 conjugate treatment groups. FIGS. 10A-10E shows the results of treatment with Compound 5 and FIG. 14 shows the results of treatment with Compound 4. Both Compounds effect a reduction in tumor size and shifted polarization of the macrophages from M2 to M1 phenotype.

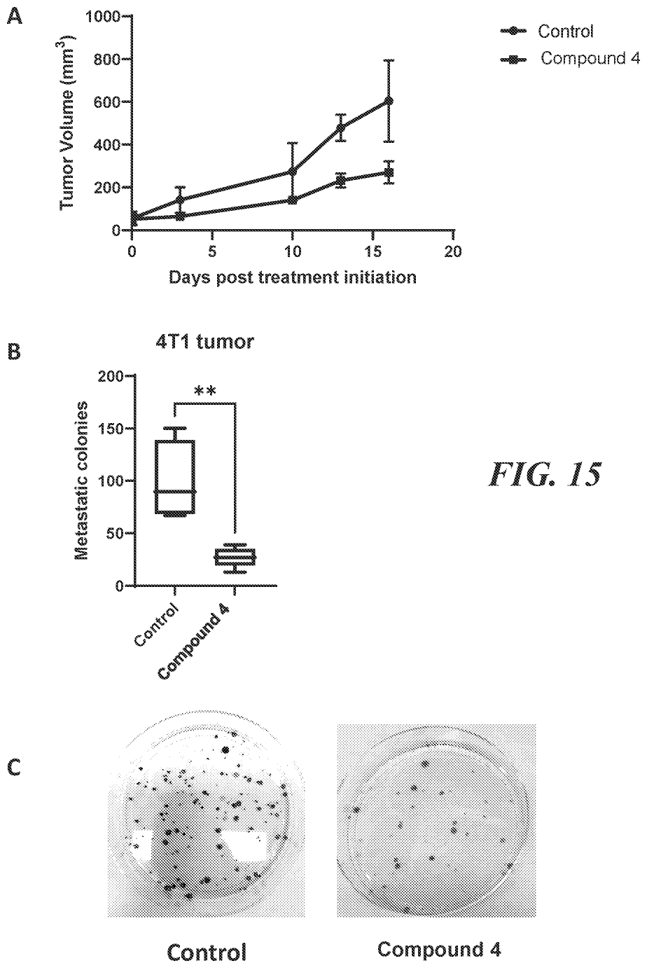

Additional 4T1 solid tumor metastatic studies were also performed. There, lung metastatic colony formation assay was carried out following the reported procedure (Cresswell et al., Folate receptor beta designates immunosuppressive tumor-associated myeloid cells that can be reprogrammed with folate-targeted drugs, Cancer Research 2021, 81(3), 671-684). 6-8 weeks old Balb/c mice were implanted with 50,000 4T1 cells in the mammary fat pad and tumors were allowed to grow until they reached ˜50 mm³. Mice were then injected intravenously with 100 μl of Folate-TLR7 conjugate (Compound 4) (50 nmole/kg) once/week. The control group received an equal volume of PBS on the same schedule. At the end of the treatment, metastasis in lung was evaluated in disease control and treatment group by co-culturing lung-digested cells with 6-thioguanine (60 mmol/L) in complete RPMI-1640 media for 10 days in petri plates. Metastatic colonies were visualized by fixing the plates with 5 mL of methanol followed by 5 mL deionized water wash and then stained with 0.03% methylene blue (see FIG. 15C). Total blue colonies from each plate were then counted.

As shown in FIG. 15A-C, treatment with the Folate-TLR7 conjugate significantly decreased the volume of the tumor and the number of metastatic colonies present within the subjects.

1. A compound represented by Formula (I): 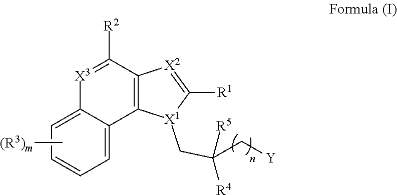
or a pharmaceutically acceptable salt thereof, wherein:
R¹, R³, R⁴, and R⁵ are each independently H, alkyl, alkoxyl, alkenyl, alkynyl, cycloalkyl, aryl, halo, heteroaryl, —COR^{2x}, 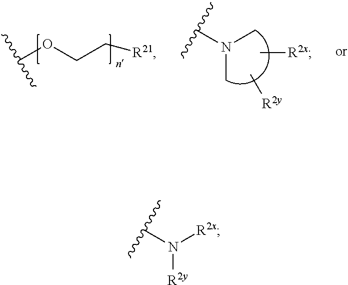
R² is H, —OH, —NH₂, —NHR^{2x}, N₃, —NH—CH₂—NH₂, —CONH₂, —SO₂NH₂, —NH—CS—NH₂, 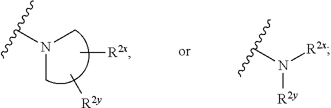
Y is H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂, 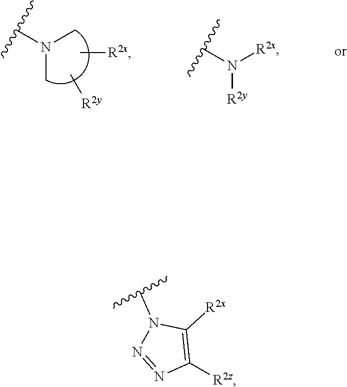
wherein:
each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl, and
each R^{2z} is independently selected from the group consisting of —NH₂, —NR^{2q}R^{2q′}, —O—R^{2q}, —SO—R^{2q}, and —COR^{2q};
wherein each R^{2q} and R^{2q′} is independently alkyl or H, 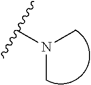 [SMILES: *N(C)C]
is a 3-10 membered N-containing non-aromatic, or mono- or bicyclic heterocycle;
R²¹ is H or alkyl;
n′ is 0-30;
wherein, in Formula I, each of X¹, X², and X³ is independently CR^{q} or N, and each R^{q} is independently hydrogen, halogen, or optionally substituted alkyl;
n is 0-30,
m is 0-4; and
wherein when n is 0, Y is not H, —OH, or —O—R^{2x}.

2. (canceled)

3. The compound of claim 1, wherein R¹ is an optionally substituted C₃-C₆ alkyl.

4. (canceled)

5. The compound of claim 1, wherein R² is —NR^{2x}R^{2y}.

6. The compound of claim 1, wherein R² is —NH₂.

7. The compound of claim 1, wherein the compound is a compound of any of the formulae: 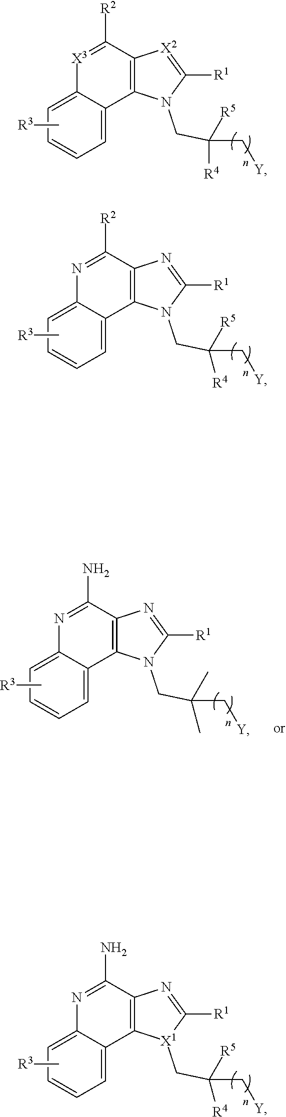
or a pharmaceutically acceptable salt thereof.

8. The compound of claim 1, wherein n is 1-3.

9-10. (canceled)

11. The compound of claim 1, wherein Y is —OH, OCH₃, —NH₂, —NHNH₂, —NHCONH₂, —SH, —SO₂NH₂, —N₃, —COOH, —COCH₃, —COOCH₃, or —CONH₂.

12. (canceled)

13. The compound of claim 1, wherein R⁴ and R⁵ are each independently C₁-C₄ alkyl.

14. (canceled)

15. The compound of claim 1, wherein the compound is a compound of any of the formulae: 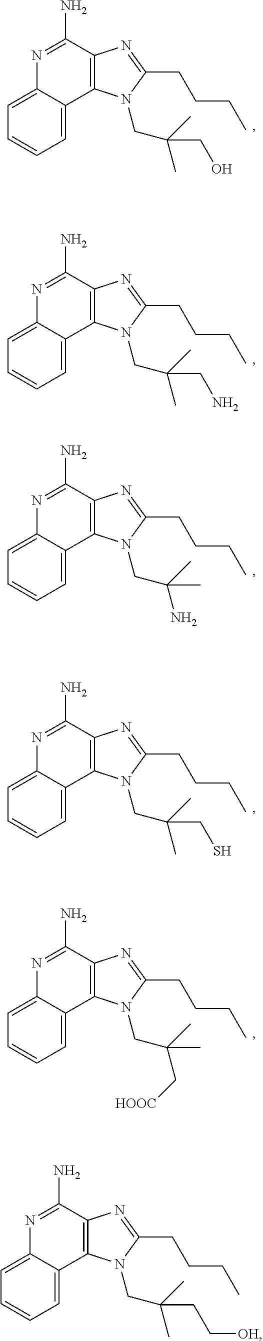 [SMILES: CCCCc1nc2c(N)nc3ccccc3c2n1CC(C)(C)CO ; CCCCc1nc2c(N)nc3ccccc3c2n1CC(C)(C)CN ; CCCCc1nc2c(N)nc3ccccc3c2n1CC(C)(C)N ; CCCCc1nc2c(N)nc3ccccc3c2n1CC(C)(C)CS ; CCCCc1nc2c(N)nc3ccccc3c2n1CC(C)(C)CC(=O)O ; CCCCc1nc2c(N)nc3ccccc3c2n1CC(C)(C)CCO] 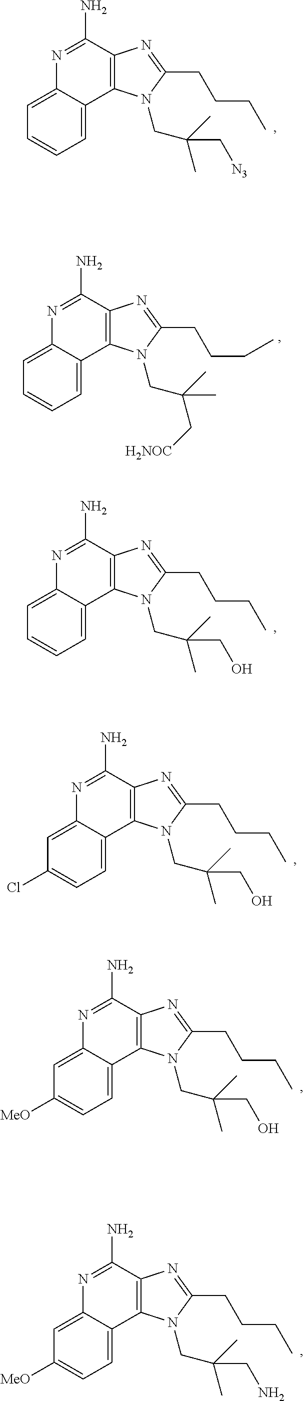 [SMILES: CCCCc1nc2c(N)nc3ccccc3c2n1CC(C)(C)CN=[N+]=[N-] ; CCCCc1nc2c(N)nc3ccccc3c2n1CC(C)(C)CC(N)=O ; CCCCc1nc2c(N)nc3ccccc3c2n1CC(C)(C)CO ; CCCCc1nc2c(N)nc3cc(Cl)ccc3c2n1CC(C)(C)CO ; CCCCc1nc2c(N)nc3cc(OC)ccc3c2n1CC(C)(C)CO ; CCCCc1nc2c(N)nc3cc(OC)ccc3c2n1CC(C)(C)CN] 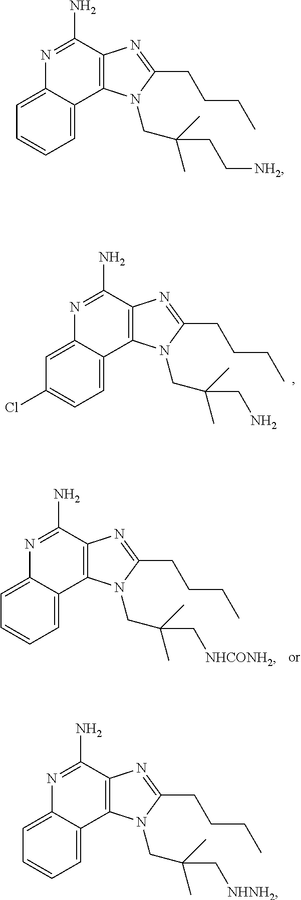
or a pharmaceutically acceptable salt thereof.

16-39. (canceled)

40. The conjugate of claim 75, wherein the linker is a non-releasable linker.

41-50. (canceled)

51. The conjugate of claim 75, wherein the linker is a bivalent linker.

52-58. (canceled)

59. A pharmaceutical composition comprising: (a) the conjugate of claim 75, or pharmaceutically acceptable salt thereof, and (b) a pharmaceutically acceptable excipient.

60. (canceled)

61. A method of treating cancer in patient in need thereof, comprising administering a therapeutically effective amount of to the patient the conjugate of claim 75 or pharmaceutically acceptable salt thereof.

62. (canceled)

63. The method of claim 61, wherein the cancer is lung cancer.

64. (canceled)

65. A method of treating a fibrotic disorder in a patient in need thereof, comprising administering to the patient a therapeutically effective amount of the conjugate of claim 75 or pharmaceutically acceptable salt thereof.

66-72. (canceled)

73. The compound of claim 1, wherein each R³ is H.

74. A radical of the compound of claim 1.

75. A conjugate, comprising:
(i) the radical of claim 74;
(ii) a linker; and
(iii) a targeting moiety that binds to a target on an immune cell;
wherein the radical of claim 74 is connected to the targeting moiety by the linker; or
a pharmaceutically acceptable salt thereof.

76. The conjugate of claim 75, wherein the linker is pegylated and/or comprises a spacer.

77. The conjugate of claim 75, wherein the targeting moiety is a hormone, an antibody, or a vitamin.

78. The conjugate of claim 75, wherein the target is a folate receptor.

79. The conjugate of claim 75, wherein the target is folate receptor beta (FR-J).

80. The conjugate of claim 75, wherein the targeting moiety is a radical of folate, 5-methyltetrahydrofolate (5-MTHF), 5-formyltetrahydrofolate (5-formyl-THF), 10-formyltetrahydrofolate (10-formyl-THF), 5,10-methylenetetrahydrofolate (5,10-methylene-THF), 5,10-methenyltetrahydrofolate (5,10-methenyl-THF), 5,10-formiminotetrahydrofolate (5,10-formimino-THF), 5,6,7,8-tetrahydrofolate (THF), or diliydrofolicacid (DHF).

81. The conjugate of claim 75, wherein the immune cell is an innate immune cell.

82. The conjugate of claim 75, wherein the immune cell is a myeloid cell.

83. The conjugate of claim 75, wherein the immune cell is a macrophage.

84. The conjugate of claim 75, wherein the immune cell is a monocyte.

85. The conjugate of claim 75, wherein the immune cell is a myeloid-derived suppressor cell (MDSC).

86. The conjugate of claim 75, wherein the immune cell is localized in a tumor microenvironment.
